(19) United States
(12) Patent Application Publication
(10) Pub. No.: US 2024/0003898 A1
(43) Pub. Date: Jan. 4, 2024

(54) METHODS FOR MEASURING DYSTROPHIN IN TISSUE SAMPLES

(71) Applicant: PFIZER INC., New York, NY (US)

(72) Inventors: Vahid FARROKHI, Andover, MA (US); Hendrick NEUBERT, Andover, MA (US); Joe PALANDRA, Andover, MA (US); Jason Michael WALSH, Andover, MA (US)

(21) Appl. No.: 18/249,752

(22) PCT Filed: Oct. 21, 2021
(86) PCT No.: PCT/IB2021/060037

§ 371 Date: Apr. 20, 2023

Related U.S. Application Data
(60) Provisional application No. 63/107,762, filed on Oct. 30, 2020.

(51) Int. Cl. G01N 33/68; G01N 33/58; G01N 1/40

(52) U.S. Cl. CPC G01N 33/6848; G01N 33/58; G01N 33/6887; G01N 1/4044; G01N 2800/2885; G01N 2333/4712

(57) ABSTRACT

Embodiments described herein provide a solution to the measurement of various dystrophins expressed in a cell or tissue. The solution to this problem is a specific and sensitive quantitative method to measure endogenous and/or exogenous dystrophin (e.g., engineered dystrophin) expression in skeletal muscle tissue, which provides a substantial benefit to drug development and monitoring of treatment.

### FIELD

The present invention generally relates to cellular biology, molecular biology, and gene therapy. In particular aspects the invention relates to procedures for assessing levels of an expressed protein, as well as related compositions, methods, and uses thereof.

### BACKGROUND

Duchenne Muscular Dystrophy (DMD) is a lethal, muscle-wasting disorder caused by a mutation in the DMD gene, resulting in the absence or reduction of the protein dystrophin (Hoffman E P, et al (1987), Cell 51(6):919-928). Dystrophin is a large 427 kDa protein with an mRNA of 14 kb composed of 79 exons (Koenig M, et al. (1987) Cell 50(3):509-517). Tissue-specific promoters and poly-A addition sites produce numerous dystrophin isoforms. Dp427m is the predominant isoform expressed in skeletal and cardiac muscle and is essential for the structural stability of muscle fibers (Doorenweerd N, et al. (2017), Sci Rep 7(1):12575). More than 60% of documented DMD mutations are deletions of one or more exons (Flanigan K M, et al. (2009), Hum Mutat 30(12):1657-1666).

Mutations that maintain the dystrophin reading frame tend to produce a truncated but partially functioning dystrophin protein that causes the milder form, Becker muscular dystrophy (BMD). BMD is rarer and more clinically heterogeneous than DMD, with an estimated prevalence of 1.53 per 100,000 males (Mah J K, et al. (2014), Neuromuscul Disord 24(6):482-491). Patients with BMD have a later onset of skeletal muscle weakness and demonstrate slower disease progression. In patients with BMD, a dystrophin level >10% appears to be sufficient to avoid a more severe disease course (van den Bergen J C, et al. (2014), J Neurol Neurosurg Psychiatry 85(7):747-753). Observations from patients with X-linked cardiomyopathy, where dystrophin is absent in cardiac tissue and reduced in skeletal muscle, suggest that full-length dystrophin levels of 30% in skeletal muscle may be sufficient to prevent muscular dystrophy (Neri M, et al. (2007), Neuromuscul Disord 17(11-12):913-918). Although, evidence from animal models suggests that the threshold to restore muscular function may be higher (>40%) (Le Guiner C, et al. (2014), Mol Ther 22(11):1923-1935), restoration of dystrophin in skeletal muscle represents a viable therapeutic approach.

Numerous strategies aimed at restoring dystrophin in muscle are being explored, including exon skipping, CRISPR, and gene therapy. One promising strategy is to use adeno-associated virus (AAV) vectors (Duan D (2018), Mol Ther 26(10):2337-2356), which have shown high-level, persistent, systemic expression in muscle cells with an apparent lack of pathogenicity (Yue Y, et al. (2008), Mol Ther 16(12):1944-1952). An obstacle to the use of AAV vectors is that the packaging capacity (˜5 kb) is much smaller than that of the dystrophin gene (2100 kb). Stemming from observations in patients with BMD, numerous engineered dystrophins have been developed. These highly truncated versions of dystrophin restore a certain level of function in preclinical mouse and canine models (Shin J H, et al. (2013), Mol Ther 21(4):750-757; Wang B, et al (2000) Proc Natl Acad Sci USA 97(25):13714-13719; Harper S Q, et al. (2002), Nat Med 8(3):253-261; and Le Guiner C, et al. (2017) Nat Commun 8:16105.). AAV9.hCK.Hopti-Dys3978.spA, a recombinant AAV9 capsid containing a human engineered dystrophin gene under control of a muscle-specific promoter, is one such therapy under investigation in a multicenter, open-label, nonrandomized, ascending-dose study in boys aged 5-12 years with DMD (ClincialTrials.gov, NCT03362502, 2019. URL //clinicaltrials.gov/ct2/show/NCT03362502 (accessed 18 Jun. 2020)).

It is essential that the effect of a candidate therapy on functional outcomes be demonstrable in clinical trials. However, these outcomes are challenging due to test-to-test variation and because changes may take over a year to become apparent. Evidence of a correlation of dystrophin expression with functional outcomes would support the use of quantified dystrophin protein for the accelerated approval of new treatments. Indeed, Exondys 51, an antisense oligonucleotide, received accelerated approval from the US Food and Drug Administration in 2016 based on the surrogate endpoint of increased dystrophin in skeletal muscle as assessed by Western blot.

Traditionally, Western blots and immunofluorescence staining have been used to evaluate dystrophin expression. These techniques are complementary; immunofluorescence provides information on the cellular localization of dystrophin and Western blots provides a semi-quantitative estimate of expression. However, reproducibility of results obtained using these techniques is less than optimal. Western blots can produce a high level of inter-laboratory variation, especially with samples nearing the lower limit of quantification (LLOQ) (Anthony K, et al. (2014), Neurology 83(22):2062-2069). This may partly be due to the absence of a reference standard that can be used across all laboratories affecting currently available dystrophin measurement techniques (Schnell F J, et al (2019), US Neurol 15(1):40-46). Other variables affecting the reproducibility of Western blots for quantification include gel overloading, membrane transfer efficiency, non-specific binding of detection antibodies and signal development. The use of different detection antibodies between laboratories further limits the comparability of Western blot results among studies and laboratories. Immunofluorescence shows less variation than Western blots; however, revertant fibers and trace dystrophin expression can complicate the interpretation of immunofluorescence images (Arechavala-Gomeza V, et al. (2010), Neuromuscul Disord 20(5):295-301; and Nicholson L V, et al (1990), Acta Neuropathol 80(3):239-250). Ligand-binding assays may suffer from a lack of high-quality reagents that are equally efficient in capturing both the endogenous and therapeutic versions of the dystrophin protein (Rup B & O'Hara D (2007), AAPS J 9(2):E148-155), which may result in biased measurements. Low abundance proteins such as dystrophin (0.002% of striated muscle) can be especially difficult to measure accurately using routine bioanalytical techniques (Hoffman et al (1987), Cell 51(6):919-928).The development of a specific and sensitive quantitative method to measure endogenous and engineered dystrophin expression in skeletal muscle tissue would therefore be of substantial benefit to drug development and monitoring of treatment.

Liquid chromatography tandem mass spectrometry (LC-MS/MS) has been established as a method for human dystrophin quantification. However, the initial method detects dystrophin levels only down to 5% relative to normal expression in healthy individuals. Thus, further technology advancements are required to enable regular and widespread use of LC-MS/MS for dystrophin quantification in clinical trials.

### SUMMARY OF THE INVENTION

Embodiments described herein provide a solution to the measurement or quantification of various dystrophins expressed in a cell or tissue. The solution to the problems described above is a specific and sensitive, quantitative method to measure endogenous dystrophin proteins and/or engineered dystrophin proteins present in a biological sample, as well as related compositions, which provides a substantial benefit to drug development and the monitoring of treatment of muscular dystrophy patients.

Certain embodiments provide antibodies or antigen binding fragments thereof, that specifically bind proteolytic fragments of dystrophin proteins, as well as uses, and associated methods thereof. Those skilled in the art will recognize or will be able to ascertain using no more than routine experimentation, many equivalents to the specific embodiments described herein. Such equivalents are intended to be encompassed by the following embodiments (E).
E1. A method for measuring the amount of dystrophin protein in a protein sample isolated from a biological sample comprising:
(i) contacting the protein sample with a first protease forming a protein digest comprising dystrophin peptides;
(ii) applying the protein digest to a dystrophin peptide affinity reagent, wherein the dystrophin peptides in the protein digest are bound to the dystrophin peptide affinity reagent, and eluting the bound dystrophin peptides forming a dystrophin peptide enriched sample containing endogenous dystrophin peptides, engineered dystrophin peptides, or endogenous dystrophin peptides and engineered dystrophin peptides; and
(iii) performing liquid-chromatography-mass spectrometry (LC/MS) on the dystrophin peptide enriched sample.
E2. The method as set forth in E1, wherein the biological sample is from a human, mouse, rat, monkey, or dog.
E3. The method as set forth in any one of E1-E2, wherein the biological sample is from a human.
E4. The method as set forth in E3, wherein the biological sample is from a human patient having Duchenne's muscular dystrophy (DMD).
E5. The method as set forth in E4, wherein the human patient having DMD has been treated with a gene therapy encoding an engineered dystrophin protein.
E6. The method as set forth in any one of E1-E5, wherein the dystrophin protein being measured is an engineered dystrophin protein.
E7. A method for measuring the amount of an engineered dystrophin protein in a protein sample isolated from a biological sample taken from a human patient having Duchenne's muscular dystrophy (DMD), said human patient having been previously treated with a gene therapy encoding the engineered dystrophin protein, the method comprising:
(i) contacting the protein sample with a first protease forming a protein digest, said protein digest comprising dystrophin peptides;
(ii) applying the protein digest to a dystrophin peptide affinity reagent, wherein the dystrophin peptides in the protein digest are bound to the dystrophin peptide affinity reagent, and eluting the bound dystrophin peptides forming a dystrophin peptide enriched sample containing engineered dystrophin peptides; and
(iii) performing liquid-chromatography-mass spectrometry (LC/MS) on the dystrophin peptide enriched sample.
E8. The method as set forth in any one of E1-E7, wherein the biological sample is a tissue sample.
E9. The method of any one of E1-E8, wherein the biological sample is a muscle tissue sample.
E10. The method as set forth in E9, wherein the muscle tissue sample is a quadricep or bicep sample.
E11. The method as set forth in any one of E1-E10, wherein the biological sample is a biopsy sample.
E12. The method as set forth in E11, wherein the biopsy sample is a needle biopsy sample.
E13. The method as set forth in E1 or E7, wherein the biological sample can be a blood sample or a blood fraction.
E14. The method as set forth in any one of E1-E13, wherein the biological sample weighs about 10 μg to about 50 mg.
E15. The method as set forth in any one of E1-E14, wherein the biological sample weighs about 100 μg to about 3 mg.
E16. The method as set forth in any one of E1-E15, wherein the biological sample weighs about 200 μg to about 2 mg.
E17. The method as set forth in any one of E1-E16, wherein the dystrophin protein being measured is an endogenous dystrophin protein.
E18. The method as set forth in any one of E1-E17, wherein the endogenous dystrophin protein has an amino acid sequence that is at least 95% identical to the amino acid sequence of SEQ ID NO:242.
E19. The method as set forth in any one of E1-E18, wherein the endogenous dystrophin protein has an amino acid sequence that is identical to the amino acid sequence of SEQ ID NO:242.
E20. The method as set forth in any one of E1-E19, wherein the method comprises measuring an engineered dystrophin protein.
E21. The method as set forth in any one of E1-E20, wherein the engineered dystrophin protein has an amino acid sequence that is at least 95% identical to an amino acid sequence selected from the group consisting of SEQ ID NO:243, SEQ ID NO:244, or SEQ ID NO:245.
E22. The method as set forth in any one of E1-E21, wherein the engineered dystrophin protein has an amino acid sequence that is identical to an amino acid sequence selected from the group consisting of SEQ ID NO:243, SEQ ID NO:244, or SEQ ID NO:245.
E23. The method as set forth in any one of E1-E22, wherein the engineered dystrophin protein has an amino acid sequence that is at least 95% identical to the amino acid sequence of SEQ ID NO:243.
E24. The method as set forth in any one of E1-E23, wherein the engineered dystrophin protein has an amino acid sequence that is identical to the amino acid sequence of SEQ ID NO:243.
E25. The method as set forth in any one of E1-E22, wherein the engineered dystrophin protein has an amino acid sequence that is at least 95% identical to the amino acid sequence of SEQ ID NO:244.
E26. The method as set forth in any one of E1-E22 or E25, wherein the engineered dystrophin protein has an amino acid sequence that is identical to the amino acid sequence of SEQ ID NO:244.
E27. The method as set forth in any one of E1-E22, wherein the engineered dystrophin protein has an amino acid sequence that is at least 95% identical to the amino acid of SEQ ID NO:245.
E28. The method as set forth in any one of E1-E22 or 27, wherein the engineered dystrophin protein has an amino acid sequence that is identical to the amino acid sequence of SEQ ID NO:245.
E29. The method as set forth in any one of E1-E28, wherein the protein sample further comprises an exogenous dystrophin reference protein.
E30. The method as set forth in any one of E1-E29, wherein the exogenous dystrophin reference protein is added to the protein sample.
E31. The methods as set forth in any one of E29-E30, wherein the exogenous dystrophin reference protein is a metabolically labeled exogenous dystrophin reference protein.
E32. The methods as set forth in any one of E29-E31, wherein the metabolically labeled exogenous dystrophin reference protein is labeled with ¹³C(6)-leucine.
E33. The methods as set forth in any one of E29-E32, wherein the exogenous dystrophin reference protein has an amino acid sequence that shares at least 95% identity with the engineered dystrophin protein.
E34. The method as set forth in any one of E29-E33, wherein the exogenous dystrophin reference protein has an amino acid sequence that is at least 95% identical to SEQ ID NO:243, SEQ ID NO:244, or SEQ ID NO:245.
E35. The method as set forth in any one of E29-E34, wherein the exogenous dystrophin reference protein has an amino acid sequence that is identical to an amino acid sequence selected from the group consisting of SEQ ID NO:243, SEQ ID NO:244, or SEQ ID NO:245.
E36. The method as set forth in any one of E29-E35, wherein the exogenous dystrophin reference protein has an amino acid sequence that is at least 95% identical to the amino acid sequence of SEQ ID NO:243.
E37. The method as set forth in any one of E29-E36, wherein the exogenous dystrophin reference protein has an amino acid sequence that is identical to the amino acid sequence of SEQ ID NO:243.
E38. The method as set forth in any one of E29-E35, wherein the exogenous dystrophin reference protein has an amino acid sequence that is at least 95% identical to the amino acid sequence of SEQ ID NO:244.
E39. The method as set forth in any one of E29-E35 or E38, wherein the exogenous dystrophin reference protein has an amino acid sequence that is identical to the amino acid sequence of SEQ ID NO:244.
E40. The method as set forth in any one of E29-E35, wherein the exogenous dystrophin reference protein has an amino acid sequence that is at least 95% identical to the amino acid of SEQ ID NO:245.
E41. The method as set forth in any one of E29-E35 or E40, wherein the exogenous dystrophin reference protein has an amino acid sequence that is identical to the amino acid sequence of SEQ ID NO:245.
E42. The method as set forth in any one of E1-E41, wherein the dystrophin peptide enriched sample comprises endogenous dystrophin peptides.
E43. The method as set forth in any one of E1-E42, wherein the dystrophin peptide enriched sample comprises engineered dystrophin peptides.
E44. The method as set forth in any one of E1-E43, wherein the dystrophin peptide enriched sample comprises exogenous dystrophin reference peptides.
E45. The method as set forth in any one of E1-E44, wherein digestion of both the endogenous dystrophin protein and the engineered dystrophin protein by the first protease yields a common dystrophin peptide that is present in both an endogenous dystrophin protein and an engineered dystrophin protein.
E46. The method as set forth in E45, wherein the common dystrophin peptide has the amino acid sequence of SLEGSDDAVLLQR (SEQ ID NO:179) or LLQVAVEDR (SEQ ID NO:200).
E47. The method as set forth in E45, wherein the common dystrophin peptide has the amino acid sequence of SLEGSDDAVLLQR (SEQ ID NO:179).
E48. The method as set forth in E45, wherein the common dystrophin peptide has the amino acid sequence of LLQVAVEDR (SEQ ID NO:200).
E49. The method as set forth in E1-48, wherein the engineered dystrophin comprises a continuous amino acid sequence that is present in the engineered dystrophin protein and not present in the endogenous dystrophin protein.
E50. The method as set forth in any one of E1-E49, wherein the engineered dystrophin-specific peptide has an amino acid sequence of SEQ ID NO:235, SEQ ID NO:236, SEQ ID NO:237, SEQ ID NO:238, SEQ ID NO:239, SEQ ID NO:240, or SEQ ID NO:241.
E51. The method as set forth in any one of E1-E50, wherein the engineered dystrophin-specific peptide has the amino acid sequence of WVLLQDQPDLAPGLTTIGASPTQTVTLVTQPVVTK (SEQ ID NO:235).
E52. The method as set forth in any one of E1-E50, wherein the engineered dystrophin-specific peptide has the amino acid sequence of LEMPSSLMLEVPTHR (SEQ ID NO:236).
E53. The method as set forth in any one of E1-E50, wherein the engineered dystrophin-specific peptide has the amino acid sequence of MGYLPVQTVLEGDNMET (SEQ ID NO:237).
E54. The method as set forth in any one of E1-E50, wherein the engineered dystrophin-specific peptide has the amino acid sequence of QSNLHSYVPSTYLTEITHVSQALLEVEQLLNAPDLCAK (SEQ ID NO:238).
E55. The method as set forth in any one of E1-E50, wherein the engineered dystrophin-specific peptide has the amino acid sequence of LEEQSDQWK (SEQ ID NO:239).
E56. The method as set forth in any one of E1-E50, wherein the engineered dystrophin-specific peptide has the amino acid sequence of MGYLPVQTVLEGDNMETDTM (SEQ ID NO:240).
E57. The method as set forth in any one of E1-E50, wherein the engineered dystrophin-specific peptide has the amino acid sequence of LQELTLER (SEQ ID NO:241).
E58. The method as set forth in E1-E57, wherein the exogenous dystrophin reference peptide is 95% identical to the amino acid sequence LEMPSSLMLEVPTHR (SEQ ID NO:236).
E59. The method as set forth in E1-E58, wherein the exogenous dystrophin reference peptide has the amino acid sequence of LEMPSSLMLEVPTHR (SEQ ID NO:236).
E60. The method as set forth in any one of E1-E59, wherein the exogenous reference peptide is metabolically labeled.
E61. The methods as set forth in E60, wherein the metabolically labeled exogenous dystrophin reference peptide is labeled with ¹³C(6)-leucine.
E62. In methods as set forth in any one of E1-E61, wherein the endogenous dystrophin peptides has an amino acid sequence of LLQVAVEDR (SEQ ID NO:200) and the engineered dystrophin peptide has an amino acid sequence of LEMPSSLMLEVPTHR (SEQ ID NO:236).
E63. The method as set forth in any one of E1-E62, wherein isolating the protein component of a biological sample includes contacting a homogenized biological sample with an organic solvent forming a protein precipitate.
E64. The method as set forth in E63, wherein the organic solvent is acetic acid, acetone, acetonitrile, dimethylformamide, dimethyl sulfoxide, dioxane, ethanol, isopropanol, methanol, 1-propanol, or tetrahydrofuran, or a combination thereof.
E65. The method as set forth in any one of E63-E64, wherein the organic solvent is acetone or acetonitrile.
E66. The method as set forth in any one of E63-E65, wherein contacting a homogenized biological sample with an organic solvent is performed at a temperature of about −10 to about 30° C.
E67. The method as set forth in any one of E63-E66, wherein contacting a homogenized biological sample with an organic solvent is performed at a temperature between about 15° C. and about 25° C.
E68. The method as set forth in any one of E63-E67, wherein contacting a homogenized biological sample with an organic solvent is performed at or about 20° C.
E69. The method as set forth in any one of E63-E68, wherein contacting a homogenized biological sample with an organic solvent is performed at a temperature of between about 18° C. and about 22° C.
E70. The method as set forth in any one of E63-E69, wherein contacting a homogenized biological sample with an organic solvent is performed at a temperature of about 20° C.
E71. The method as set forth in any one of E1-E70, wherein the biological sample is homogenized in a lysis buffer.
E72. The method as set forth in E71, wherein the lysis buffer is a Radio Immuno Precipitation Assay (RIPA), TER I, TER II, NP40, T-PER, or N-PER lysis buffer.
E73. The method as set forth in any one of E71-72, wherein the lysis buffer includes a detergent.
E74. The method as set forth in E73, wherein the detergent is at a concentration of about 0.1 to about 15 wt %.
E75. The method as set forth in any one of E73-E74, wherein the detergent is at a concentration of about 2 to about 8 wt %.
E76. The method as set forth in any one of E73-E75, wherein the detergent is SDS.
E77. The method as set forth in E76, wherein the lysis buffer comprises about 1 to about 10% SDS.
E78. The method as set forth in any one of E76-E77, wherein the lysis buffer comprises about 3 to about 8 wt % SDS.
E79. The method as set forth in any one of E76-E78, wherein the lysis buffer comprises about 5 wt % SDS.
E80. The method as set forth in any one of E76-E79, wherein the lysis buffer is a RIPA buffer.
E81. The method as set forth in E80, wherein the RIPA buffer contains about 10 to about 50 mM Tris-HCl, about 100 to about 200 mM NaCl, about 0.5 to about 1.% NP40, about 1 to about 2% sodium deoxycholate and about 0.1% SDS.
E82. The method as set forth in any one of E80-E81, wherein the RIPA buffer contains about 25 mM Tris·HCl, about 150 mM NaCl, about 1 wt % NP40, about 0.1 to about 1 wt % sodium deoxycholate, and about 5 wt % SDS.
E83. The method as set forth in any one of E71-E82, wherein the lysis buffer contains at least one protease inhibitor.
E84. The method as set forth in any one of E71-E83, wherein the lysis buffer contains a plurality of protease inhibitors.
E85. The method as set forth in any one of E71-E84, wherein the protease inhibitor(s) are individually and independently at a concentration of about 0.5 to about 2 wt %.
E86. The method as set forth in any one of E83-E85 wherein the protease inhibitor(s) are one or more of aprotinin, bestatin, E-64, leupeptin, and/or pepstatin A.
E87. The method as set forth in any one of E1-E86, wherein the first protease is a serine protease, threonine protease, cysteine protease, aspartate protease, glutamic acid protease, or metalloprotease.
E88. The method as set forth in any one of E1-E87, wherein the first protease is a serine protease.
E89. The method as set forth in any one of E1-E88, wherein the first protease is trypsin.
E90. The method as set forth in E1-E89, wherein the first protease is tosyl phenyl chloromethyl ketone (TPCK) treated trypsin.
E91. The method as set forth in any one of E1-E90, wherein the first protease is at a concentration of about 10 ng/μL to about 1 μg/μL.
E92. The method as set forth in any one of E1-E91, wherein the first protease is at a concentration of about 10 ng/μL to about 100 ng/μL.
E93. The method as set forth in any one of E1-E92, wherein the first protease is at a concentration of about 50 ng/μL.
E94. The method as set forth in any one of E1-E93, wherein the first protease is at a concentration of about 50 ng/μL.
E95. The method as set forth in any one of E1-E94, wherein the first or second protease is provided in a protease buffer, and the protease buffer comprises at least a chaotropic agent, organic solvent and buffer salts.
E96. The method as set forth in E95, wherein the protease buffer comprises urea, acetonitrile and phosphate buffered saline.
E97. The method as set forth in any one of E95-E96, wherein the protease buffer comprises urea at a concentration from about 0.05 M to about 4 M.
E98. The method as set forth in any one of E95-E97, wherein the protease buffer comprises acetonitrile at a concentration from about 1% to about 25%.
E99. The method as set forth in any one of E95-E98, wherein the protease buffer comprises about 0.8M urea and about 10% acetonitrile in phosphate buffered saline.
E100. The method as set forth in any one of E1-E99, wherein the peptide sample is treated with a reducing agent.
E101. The method as set forth in any one of E1-E100, wherein the reducing agent is selected from the group consisting of dithiothreitol (DTT), 2-mercaptoethanol (2-ME), and Tris(2-carboxyethyl) phosphine hydrochloride (TCEP).
E102. The method as set forth in any one of E1-E101, wherein the reducing agent is DTT.
E103. The method as set forth in any one of E1-E102, wherein the reducing agent concentration is about 50 mM to about 250 mM in the peptide sample.
E104. The method as set forth in any one of E1-E103, wherein the peptide sample has a DTT concentration of about 150 mM.
E105. The method as set forth in any one of E1-E105, wherein the reducing agent concentration is about 135 mM to about 165 mM in the peptide sample.
E106. The method as set forth in any one of E1-E105, wherein the reducing agent concentration is about 150 mM in the peptide sample.
E107. The method as set forth in any one of E1-E106, wherein the peptide sample is treated with an alkylating agent.
E108. The method as set forth in any one of E1-E107, wherein the alkylating agent is Iodoacetamide (IAA), methyl methanethiosulfonate, or N-ethylmaleimide.
E109. The method as set forth in any one of E1-E108, wherein the alkylating agent is IAA.
E110. The method as set forth in any one of E1-E109, wherein the alkylating agent is at a concentration of about 20 mM to about 500 mM in the peptide sample.
E111. The method as set forth in any one of E1-E110, wherein the alkylating agent is at a concentration of about 200 mM to about 400 mM in the peptide sample.
E112. The method as set forth in any one of E1-E111, wherein the alkylating agent is at a concentration of about 300 mM in the peptide sample.
E113. The method as set forth in any one of E1-E112, wherein the alkylating agent is at a concentration of about 270 mM to about 330 mM in the peptide sample.
E114. The method as set forth in any one of E1-E113, wherein the alkylating agent is at a concentration of about 300 in the peptide sample.
E115. The method as set forth in any one of E1-E114, wherein the peptide sample is treated with a second protease.
E116. The method as set forth in any one of E1-E115, wherein the second protease is the same as the first protease.
E117. The method as set forth in any one of E1-E116, wherein the second protease is at a concentration of about 10 to about 1000 ng/μL.
E118. The method as set forth in any one of E1-E117, wherein the second protease is at a concentration of about 10 to about 100 ng/μL.
E119. The method as set forth in any one of E1-E118, wherein the second protease is at a concentration of about 50 ng/μL.
E120. The method as set forth in any one of E1-E119, wherein the second protease is at a concentration of about 45 to about 55 ng/μL.
E121. The method as set forth in any one of E1-E120, wherein the second protease is at a concentration of about 50 ng/μL.
E122. The method as set forth in any one of E1-E121, wherein the second protease is TPCK trypsin.
E123. The method as set forth in any one of E1-E122, wherein the dystrophin peptide affinity reagent binds at least one dystrophin peptide in the peptide sample.
E124. The method as set forth in any one of E1-E123, comprising more than one dystrophin peptide affinity reagent, such that two or more different dystrophin peptides can be captured from the peptide sample.
E125. The method as set forth in any one of E1-E124, wherein the bound dystrophin peptides are from an endogenous dystrophin protein, an engineered dystrophin protein, and/or an exogenous dystrophin reference protein.
E126. The method as set forth in any one of E1-E125, wherein the dystrophin peptide affinity reagent is coupled to a column matrix forming a dystrophin peptide affinity column.
E127. The method as set forth in E126 where the dystrophin peptide affinity column is capable of specifically binding 1, 2, or more distinct dystrophin peptides.
E128. The method as set forth in any one of E1-E127, wherein the dystrophin peptide affinity column binds a common dystrophin peptide that is present in both the endogenous dystrophin protein and the engineered dystrophin protein.
E129. The method as set forth in any one of E1-E128, wherein the dystrophin peptide affinity column binds an engineered dystrophin specific peptide that is present in the engineered dystrophin protein and not present in the endogenous dystrophin protein.
E130. The method as set forth in any one of E1-E129, wherein the dystrophin peptide affinity reagent comprises an anti-dystrophin peptide antibody.
E131. The method as set forth in any one of E1-E130, wherein the anti-dystrophin peptide antibody is raised against an endogenous dystrophin peptide.
E132. The method as set forth in any one of E1-E130, wherein the anti-dystrophin peptide antibody is raised against an engineered dystrophin peptide.
E133. The method as set forth in any one of E1-E132, wherein the anti-dystrophin peptide antibody is raised against a peptide comprising an amino acid sequence selected from the group consisting of SEQ ID NO:235, SEQ ID NO:236, SEQ ID NO:237, SEQ ID NO:238, SEQ ID NO:239, SEQ ID NO:240, or SEQ ID NO:241.
E134. The method as set forth in any one of E1-E133, wherein the anti-dystrophin peptide antibody is raised against a peptide comprising the amino acid sequence of SEQ ID NO:235.
E135. The method as set forth in any one of E1-E133, wherein the anti-dystrophin peptide antibody is raised against a peptide comprising the amino acid sequence of SEQ ID NO:236.
E136. The method as set forth in any one of E1-E133, wherein the anti-dystrophin peptide antibody is raised against a peptide comprising the amino acid sequence of SEQ ID NO:237.
E137. The method as set forth in any one of E1-E133, wherein the anti-dystrophin peptide antibody is raised against a peptide comprising the amino acid sequence of SEQ ID NO:238.
E138. The method as set forth in any one of E1-E133, wherein the anti-dystrophin peptide antibody is raised against a peptide comprising the amino acid sequence of SEQ ID NO:239.
E139. The method as set forth in any one of E1-E133, wherein the anti-dystrophin peptide antibody is raised against a peptide comprising the amino acid sequence of SEQ ID NO:240.
E140. The method as set forth in any one of E1-E133, wherein the anti-dystrophin peptide antibody is raised against a peptide comprising the amino acid sequence of SEQ ID NO:241.
E141. The method as set forth in any one of E1-E133, wherein the anti-dystrophin peptide antibody is raised to common dystrophin peptide.
E142. The method as set forth in any one of E1-E130 or 141, wherein the anti-dystrophin peptide antibody is raised against a peptide comprising the amino acid sequence of LLQVAVEDR (SEQ ID NO:200).
E143. The method as set forth in any one of E1-E142, wherein the anti-dystrophin peptide antibody is a polyclonal antibody.
E144. The method as set forth in any one of E1-E143, wherein the anti-dystrophin peptide antibody is a monoclonal antibody.
E145. The method as set forth in any one of E1-E144, wherein the LC/MS includes a reversed phase nano liquid chromatography.
E146. The method as set forth in any one of E1-E145, wherein the mass spectrometer is a triple quadrupole mass spectrometer configured for multiple reaction monitoring (MRM).
E147. The method as set forth in any one of E1-E146, wherein the methods further include homogenizing the biological sample prior to isolating the protein component of the biological sample.
E148. The method of as set forth in any one of E145-E147, wherein the reverse phase nanoflow chromatography column is a C18 column having 3 μm particle size, 100 Å pore size, and dimensions of 75 μm×15 cm.
E149. The method as set forth in any one of E1-E148, wherein the mass spectrometry includes electrospray ionization of the dystrophin peptide and detection of ionized peptide fragments.
E150. The method as set forth in any one of E1-E149, wherein the ionized peptide fragments are detected using triple quadrupole mass spectrometer in multiple reaction monitoring (MRM) mode.
E151 The method as set forth in any one of E1-E150, wherein analyzing the dystrophin peptide further comprises quantitating the dystrophin peptides using a peptide calibration curve.
E152. An anti-dystrophin peptide antibody raised against a peptide comprising an amino acid sequence selected from the group consisting of SEQ ID NO:235, SEQ ID NO:236, SEQ ID NO:237, SEQ ID NO:238, SEQ ID NO:239, SEQ ID NO:240, or SEQ ID NO:241.
E153. The antibody as set forth in E152, wherein the anti-dystrophin peptide antibody is raised against a peptide comprising the amino acid sequence of SEQ ID NO:235.
E154. The antibody as set forth in E152, wherein the anti-dystrophin peptide antibody is raised against a peptide comprising the amino acid sequence of SEQ ID NO:236.
E155. The antibody as set forth in E152, wherein the anti-dystrophin peptide antibody is raised against a peptide comprising the amino acid sequence of SEQ ID NO:237.
E156. The antibody as set forth in E152, wherein the anti-dystrophin peptide antibody is raised against a peptide comprising the amino acid sequence of SEQ ID NO:238.
E157. The antibody as set forth in E152, wherein the anti-dystrophin peptide antibody is raised against a peptide comprising the amino acid sequence of SEQ ID NO:239.
E158. The antibody as set forth in E152, wherein the anti-dystrophin peptide antibody is raised against a peptide comprising the amino acid sequence of SEQ ID NO:240.
E159. The antibody as set forth in E152, wherein the anti-dystrophin peptide antibody is raised against a peptide comprising the amino acid sequence of SEQ ID NO:241.
E160. The antibody as set forth in any one of E152-E159, wherein the anti-dystrophin peptide antibody is raised against a common dystrophin peptide.
E161. The antibody as set forth in any one of E152-E160, wherein the anti-dystrophin peptide antibody is raised against a peptide comprising the amino acid sequence of LLQVAVEDR (SEQ ID NO:200).
E162. The antibody as set forth in any one of E152-E161, wherein the anti-dystrophin peptide antibody is a polyclonal antibody.
E163. The antibody as set forth in any one of E152-E161, wherein the anti-dystrophin peptide antibody is a monoclonal antibody.
E164. Use of an antibody in a method as set forth in any one of E1-E151.
E165. A peptide comprising the amino acid sequence selected from the group consisting of SEQ ID NO:235, SEQ ID NO:236, SEQ ID NO:237, SEQ ID NO:238, SEQ ID NO:239, SEQ ID NO:240, and SEQ ID NO:241.
E166. Use of a peptide of E165 in a method of generating antibodies to an engineered dystrophin protein.
E167. A kit comprising compositions, peptides, antibodies, reagents, devices or for use in methods as set forth in any one of E1-E166.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

### BRIEF DESCRIPTION OF THE DRAWINGS

FIGS. 1A-1B. (1A) Immunoaffinity LC-MS/MS workflow and (1B) dystrophin engineered dystrophin peptide sequences.

FIGS. 2A-2C. The effect of SDS on percentage dystrophin protein extraction from normal human tissue. (2A) RIPA (0.1% SDS) vs. TER-1 (no SDS); (2B) Effect of the proportion of SDS on percentage dystrophin protein extraction; (2C) Percentage of dystrophin protein re-extraction from debris relative to 5% SDS.

FIG. 3. Relative concentration of dystrophin is displayed on the left y-axis and absolute concentration of dystrophin (fmol/mg) on the right y-axis. The lower and upper hinges of the boxplot correspond to the first and third quartiles (the 25^{th} and 75^{th} percentiles); the upper whisker extends from the hinge to the largest value no further than 1.5*IQR (inter-quartile range) from the hinge, and the converse is the case for the lower whiskers. Data beyond the end of the whiskers are outliers and are plotted individually. Bars are the 95% confidence intervals for the mean in each group (diamonds) based on bootstrap method.

FIGS. 4A-4F. Representative common dystrophin peptide (LLQVAVEDR (SEQ ID NO:200)) extracted ion chromatograms from age-matched normal, BMD and DMD muscle biopsies. (4A) Normal sample 9, 102.9% of mean normal; (4B) BMD sample 34, 31.2% of mean normal; (4C) DMD sample 50, 5.1% of mean normal; (4D) Normal sample 16, 103.3% of mean normal; (4E) BMD sample 40, 32.6% of mean normal; (4F) DMD sample 59, 7.3% of mean normal.

FIGS. 5A-5L. LLOQ, lower limit of quantification; QCH, high concentration quality control sample; QCL, low concentration quality control sample; RT, retention time; SIL, stable isotope labelled (peptide); ULOQ, upper limit of quantification.

FIGS. 6A-6B. IA-LC-MS/MS analysis of biceps femoris tissue samples from dystrophin-deficient (DMD^{mdx}) rats treated with increasing doses of AAV9.hCK.Hopti-Dys3978.spA at 6 month post-dose, showed a dose-dependent increase in engineered dystrophin protein expression as measured by two peptides (6A) LLQVAVEDR (SEQ ID NO:200) and (6B) SLEGSDDAVLLQR (SEQ ID NO:179). Maximum engineered dystrophin protein expression was observed in the 1×10¹⁴ vg/kg dose group. Expression of SLEG (engineered dystrophin) and LLQV (common dystrophin peptide) were comparable, with a maximum total dystrophin protein also observed in the 1×10¹⁴ vg/kg dose group.

FIG. 7. The effect of OCT on assay performance as assessed in normal human tissue.

FIGS. 8A-8B. (8A) Expression of dystrophin in males and females, normal population. (8B) Total protein content in age-matched normal, BMD and DMD muscle biopsies.

### DETAILED DESCRIPTION OF THE INVENTION

The accurate quantification of low abundance proteins, such as dystrophin, is challenging but essential for the development and validation of gene therapies aimed at restoring cellular dystrophin. Herein is described an immunoaffinity liquid chromatography tandem mass spectrometry (IA LC-MS/MS) method of high accuracy, precision and sensitivity with application in preclinical studies, human clinical trials, and as a therapeutic tool to quantify expression levels of the therapeutic in patients for the quantification of dystrophin. This method has several advantages over traditional methods. The traditional methods can be subject to high levels of variation and may be of limited sensitivity.

The methods of the invention may be useful in analyzing the expression of dystrophin expression in a biological sample. The methods of the invention may be useful in quantifying the expression of dystrophin expression in a biological sample. The methods of the invention may be useful in analyzing the expression of engineered dystrophin expression in a biological sample. The methods of the invention may be useful in quantifying the expression of engineered dystrophin expression in a biological sample. The methods of the invention may be useful for preparing a peptide sample for analysis of dystrophin expression in a biological or tissue sample.

In certain aspects, the expression level of endogenous dystrophin or engineered dystrophin in DMD, BMD, and healthy (normal) skeletal muscle tissue can be examined by measuring, quantitating, or assessing peptide fragments of dystrophin proteins in a sample. In certain aspects, anti-peptide antibodies are employed to isolate dystrophin peptides for analysis. The use of anti-peptide antibodies eliminates many of the challenges associated with anti-protein antibodies, including a lack of capture efficiency and protein denaturation during extraction (Neubert et al., Bioanalysis. 8, 1551-1555 (2016)). Embodiments of the methods described herein allow for a large number of samples to be run at once including calibrants and quality control (QC) standards, eliminating the need for multiple assays. Samples for different analytical assays, such as immunofluorescence, can be taken from the same tissue block, further reducing the potential for variation. The use of an internal standard allows the quantification of dystrophin at the femtomole level and is able to detect dystrophin in DMD muscle samples as low as 0.4% relative to healthy human muscle.

In some aspects, the invention provides methods for the measurement, quantification, and/or assessment of dystrophin proteins expressed in or by a cell or tissue. In some aspects, the biological sample is from a human, mouse, rat, monkey, or dog. In some aspects, the biological sample is from a human. The human may be a human patient having Duchenne's muscular dystrophy (DMD). The human patient having DMD may have been treated with a gene therapy encoding an engineered dystrophin protein or other DMD therapy. The dystrophin protein can be an endogenous dystrophin protein or an engineered dystrophin protein. In particular, the engineered dystrophin protein may be engineered to provide a gene therapy treatment for DMD, BMD, or dystrophin deficiencies.

The invention provides for methods for measuring the amount of dystrophin protein in a protein sample isolated from a biological sample comprising: (i) contacting the protein sample with a first protease forming a protein digest comprising dystrophin peptides; (ii) applying the protein digest to a dystrophin peptide affinity reagent, wherein the dystrophin peptides in the protein digest are bound to the dystrophin peptide affinity reagent, and eluting the bound dystrophin peptides forming a dystrophin peptide enriched sample containing endogenous dystrophin peptides, engineered dystrophin peptides, or endogenous dystrophin peptides and engineered dystrophin peptides; and (iii) performing liquid-chromatography-mass spectrometry (LC/MS) on the dystrophin peptide enriched sample. The dystrophin peptides may comprise engineered dystrophin peptides (generated by the proteolytic degradation of engineered dystrophin protein expressed by a transgene), endogenous dystrophin peptides (generated by the proteolytic degradation of endogenous dystrophin), and/or common dystrophin peptides (peptides common to or present in the digests of both endogenous dystrophin protein and engineered dystrophin protein).

In certain embodiments a dystrophin peptide comprises, consist essentially of, or consist of at least, at most, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, contiguous amino acids, including all values and ranges there between, of one or more of SEQ ID NO:242, SEQ ID NO:243, SEQ ID NO:244, or SEQ ID NO:245. In certain aspects, these dystrophin peptides can have 1, 2, 3, 4, 5, 6, 7, 8, 9, 10, 11, 12, or more amino acid substitutions at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of the peptide. In some embodiments, the dystrophin peptide can have 1, 2, 3, 4, 5, 6, 7, 8, 9, 10, 11, 12, or more amino acid substitution(s) at one or more of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of any of SEQ ID NOs: 1 to 241 with any of the following amino acids: alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), or valine (val, V).

The measurement, quantification, or assessment of dystrophin protein expression is by measuring, quantifying, or assessing the amount of two or more proteolytic fragment(s) of a dystrophin protein (dystrophin peptide(s)) common to an endogenous and engineered dystrophin protein (i.e., a common dystrophin peptide which is present in both an endogenous dystrophin protein and an engineered dystrophin protein), specific to an endogenous dystrophin protein, or specific to an engineered dystrophin protein. The invention provides methods of measurement, quantification, or assessment of endogenous dystrophin protein. The invention provides methods of measurement, quantification, or assessment of engineered dystrophin protein.

In some aspects, the invention provides methods of measurement. In some aspects, the measurement is quantification. In particular, the invention provides methods of quantification of engineered dystrophin protein in a biological sample from a DMD patient treated with gene therapy encoding an engineered dystrophin protein.

The invention provides a method for measuring, quantifying, or assessing the amount of an engineered dystrophin protein in a protein sample isolated from a biological sample taken from a human patient having Duchenne's muscular dystrophy (DMD), said human patient having been previously treated with a gene therapy encoding the engineered dystrophin protein, the method comprising: (i) contacting the protein sample with a first protease forming a protein digest, said protein digest comprising dystrophin peptides; (ii) applying the protein digest to a dystrophin peptide affinity reagent, wherein the dystrophin peptides in the protein digest are bound to the dystrophin peptide affinity reagent, and eluting the bound dystrophin peptides forming a dystrophin peptide enriched sample containing engineered dystrophin peptides; and (iii) performing liquid-chromatography-mass spectrometry (LC/MS) on the dystrophin peptide enriched sample.

An endogenous dystrophin protein can have an amino acid sequence that is at least 80, 85, 90, 91, 92, 93, 94, 95, 96, 97, 98, 99, or 100% identical, including al values and ranges there between, to the amino acid sequence of SEQ ID NO:242 or any natural variant of the dystrophin gene or protein. In certain aspects, the endogenous dystrophin protein has an amino acid sequence that is identical to the amino acid sequence of SEQ ID NO:242. In certain aspects, endogenous dystrophin peptides can include peptides having the amino acid sequence of SEQ ID NO:1 to SEQ ID NO:234, which are present in endogenous dystrophin having the amino acid sequence of SEQ ID NO:242. Similar peptide profiles can be determined for other endogenous dystrophin proteins that are variants of SEQ ID NO:242.

In some aspects, the invention provides a method for measuring the amount of engineered dystrophin protein in a protein sample isolated from a biological sample, wherein the biological sample is from a human patient having DMD having been treated with a gene therapy encoding an engineered dystrophin protein comprising SEQ ID NO:242, said method comprising:
(i) contacting the protein sample with a first protease forming a protein digest comprising dystrophin peptides;
(ii) applying the protein digest to a dystrophin peptide affinity reagent, wherein the dystrophin peptides in the protein digest are bound to the dystrophin peptide affinity reagent, and eluting the bound dystrophin peptides forming a dystrophin peptide enriched sample containing endogenous dystrophin peptides, engineered dystrophin peptides, or endogenous dystrophin peptides and engineered dystrophin peptides; and
(iii) performing liquid-chromatography-mass spectrometry (LC/MS) on the dystrophin peptide enriched sample; and wherein the dystrophin peptide affinity reagent comprises an antibody raised against an engineered dystrophin peptide comprising SEQ ID NO:236.

In some aspects, the engineered dystrophin protein has an amino acid sequence of a recombinant therapeutic dystrophin protein. An engineered dystrophin protein can have an amino acid sequence that is at least 80, 85, 90, 91, 92, 93, 94, 95, 96, 97, 98, 99, or 100% identical, including all values and ranges there between, to an engineered dystrophin protein selected from the group consisting of SEQ ID NO:243, SEQ ID NO:244, and SEQ ID NO:245. In certain aspects, the engineered dystrophin protein has an amino acid sequence that is identical to an amino acid sequence of SEQ ID NO:243, SEQ ID NO:244, or SEQ ID NO:245.

The engineered dystrophin protein can have an amino acid sequence that is at least 95% identical to the amino acid sequence of SEQ ID NO:243. In certain aspects, the engineered dystrophin protein can have an amino acid sequence that is identical to the amino acid sequence of SEQ ID NO:243. In certain aspects, the engineered dystrophin protein of SEQ ID NO:243 includes or can be proteolyzed to peptides having the amino acid sequence of SEQ ID NO: 235, SEQ ID NO:236, and SEQ ID NO:237. Peptides having the amino acid sequence of SEQ ID NO: 235, SEQ ID NO:236, and SEQ ID NO:237 may be used to generate antibodies for the detection, measurement or quantification of the engineered dystrophin protein of SEQ ID NO:243. Peptides having the amino acid sequence of SEQ ID NO:179 or SEQ ID NO:200 may be used to generate antibodies to detect common dystrophin peptides in biological samples from patients treated with vectors encoding SEQ ID NO:243.

The engineered dystrophin protein can have an amino acid sequence that is at least 95% identical to the amino acid sequence of SEQ ID NO:244. In certain aspects, the engineered dystrophin protein has an amino acid sequence that is identical to the amino acid sequence of SEQ ID NO:244. In certain aspects, engineered dystrophin protein of SEQ ID NO:244 includes or can be proteolyzed to peptides having the amino acid sequence of SEQ ID NO: 238, SEQ ID NO:239, and SEQ ID NO:240. Peptides having the amino acid sequence of SEQ ID NO: 238, SEQ ID NO:239, and SEQ ID NO:240 may be used to generate antibodies for the detection, measurement or quantification of the engineered dystrophin protein of SEQ ID NO:244. A peptide having the amino acid sequence of SEQ ID NO:200 may be used to generate antibodies to detect common dystrophin peptides in biological samples from patients treated with vectors encoding SEQ ID NO:244.

The engineered dystrophin protein can have an amino acid sequence that is at least 95% identical to the amino acid of SEQ ID NO:245. In certain aspects, the engineered dystrophin protein has an amino acid sequence that is identical to the amino acid sequence of SEQ ID NO:245. In certain aspects, engineered dystrophin protein of SEQ ID NO:245 includes or can be proteolyzed to peptides having the amino acid sequence of SEQ ID NO: 240 and SEQ ID NO:241. Peptides having the amino acid sequence of SEQ ID NO:240 and/or SEQ ID NO:241 may be used to generate antibodies for the detection, measurement or quantification of the engineered dystrophin protein of SEQ ID NO:245. Peptides having the amino acid sequence of SEQ ID NO:200 may be used to generate antibodies to detect common dystrophin peptides in biological samples from patients treated with vectors encoding SEQ ID NO:245.

Peptide Selection

The peptide sequence LLQVAVEDR (SEQ ID NO:200) is present in endogenous dystrophin and some species of engineered dystrophin (e.g. SEQ ID NO:243). It is expressed in human, cynomolgus, rattus and mus, but not in canis species. Upon digestion, this peptide is produced in an equimolar fashion by both endogenous dystrophin and engineered dystrophin, i.e., one mole of endogenous dystrophin or engineered dystrophin produces 1 mole of the peptide LLQVAVEDR (SEQ ID NO:200). Therefore, it is viable for use in clinical and preclinical investigations as a combined molar measure of endogenous dystrophin and engineered dystrophin, overcoming the challenges associated with Western blot-based percent normal calculations. The engineered dystrophin peptide having the amino acid sequence LEMPSSLMLEVPTHR (SEQ ID NO:236) is present in engineered dystrophin protein of SEQ ID NO:243 only and does not occur in the proteome of humans or any preclinical species. Similarly, the engineered dystrophin peptides having the the amino acid sequence SEQ ID NO:238, SEQ ID NO:239, and SEQ ID NO:240 are present in engineered dystrophin protein of SEQ ID NO:244 only and do not occur in the proteome of humans or any preclinical species. Similarly, the engineered dystrophin peptides having the the amino acid sequence SEQ ID NO:240 and SEQ ID NO:241 are present in engineered dystrophin protein of SEQ ID NO:245 only and do not occur in the proteome of humans or any preclinical species. In some aspects, the engineered dystrophin peptides span a junction between a dystrophin hinge region and rod domain created by deletion of large portion of the central rods and hinges. In some aspects, the engineered dystrophin peptides have an antigenicity score of at least 2.0. In some aspects, the engineered dystrophin peptides may be used in both clinical and preclinical assessment of transgene protein expression. SLEGSDDAVLLQR (SEQ ID NO:179) is present in human endogenous dystrophin and engineered dystrophin and is suitable for use in preclinical investigations as a combined measure of engineered dystrophin, or if needed, in the clinical assay as a measure of total dystrophin (endogenous dystrophin plus engineered dystrophin). In a rat study of AAV9.hCK.Hopti-Dys3978.spA, as presented herein below, SEQ ID NO:179 may therefore used as an engineered dystrophin peptide.

In some aspects, the invention provides methods to measure, quantify, or assess endogenous, engineered, or endogenous and engineered dystrophin peptides. In some aspects, the invention provides methods to measure, quantify, or assess at least two peptides. Peptide LLQVAVEDR (SEQ ID NO:200), which is specific to both endogenous dystrophin and engineered dystrophin and peptide, and LEMPSSLMLEVPTHR (SEQ ID NO:236), which is specific to an engineered dystrophin only. In some aspects, the invention provides methods to measure, quantify, or assess at least two peptides—the peptide SLEGSDDAVLLQR (SEQ ID NO:179), which is specific to both endogenous dystrophin and engineered dystrophin, and the peptide LEMPSSLMLEVPTHR (SEQ ID NO:236), which is specific to an engineered dystrophin only. The range of quantitation in muscle lysate is 20.0 fmol/mL to 3333 fmol/mL for all three peptides.

In some aspects, the digestion of both the endogenous dystrophin protein and the engineered dystrophin protein by the first protease yields a common dystrophin peptide that is present in both an endogenous dystrophin protein and an engineered dystrophin protein. The common dystrophin peptide may comprise or consist of the amino acid sequence of LLQVAVEDR (SEQ ID NO:200). In certain aspects, the common dystrophin peptide may comprise or consist of the amino acid sequence of SLEGSDDAVLLQR (SEQ ID NO:179).

The engineered dystrophin peptide may comprise a contiguous amino acid sequence that is present in the engineered dystrophin protein and is not contiguously present in the endogenous dystrophin protein. The engineered dystrophin peptide may comprise or consist of an amino acid sequence selected from the group consisting of SEQ ID NO:235, SEQ ID NO:236, SEQ ID NO:237, SEQ ID NO:238, SEQ ID NO:239, SEQ ID NO:240, and SEQ ID NO:241.

In some aspects, the engineered dystrophin peptide comprises or consists of the amino acid sequence of WVLLQDQPDLAPGLTTIGASPTQTVTLVTQPVVTK (SEQ ID NO:235). In some aspects, the engineered dystrophin peptide comprises or consists of the amino acid sequence of LEMPSSLMLEVPTHR (SEQ ID NO:236). In some aspects, the engineered dystrophin peptide comprises or consists of the amino acid sequence of MGYLPVQTVLEGDNMET (SEQ ID NO:237). In some aspects, the engineered dystrophin peptide comprises or consists of the amino acid sequence of QSNLHSYVPSTYLTEITHVSQALLEVEQLLNAPDLCAK (SEQ ID NO:238). In some aspects, the engineered dystrophin peptide comprises or consists of the amino acid sequence of LEEQSDQWK (SEQ ID NO:239). In some aspects, the engineered dystrophin peptide comprises or consists of the amino acid sequence of MGYLPVQTVLEGDNMETDTM (SEQ ID NO:240). In some aspects, the engineered dystrophin peptide comprises or consists of the amino acid sequence of LQELTLER (SEQ ID NO:241).

Internal Standard SILAC Engineered Dystrophin.

In certain aspects, an exogenous dystrophin reference protein can be introduced during sample processing. An exogenous dystrophin reference protein can be isotopically labeled, for example using stable isotope labeling using amino acids in cell culture (SILAC). SILAC exogenous dystrophin reference protein is a recombinant labeled engineered dystrophin that is metabolically labeled with stable isotope ¹³C(6)-leucine added to the cell culture. SILAC exogenous dystrophin reference protein differs from engineered dystrophin only in ¹³C stable isotopes on leucine residues but otherwise has the same chemical properties as the engineered dystrophin and the two behave identically during sample preparation procedure. The tryptic peptides generated from proteolytic digestion of SILAC exogenous dystrophin reference protein that contain the labeled leucine have a mass shift of 6 Da (per labeled leucine) from the light peptide counterpart. SILAC exogenous dystrophin reference protein is spiked in equal amounts into all the sample lysates including unknowns, standards and quality controls in an analysis batch. Such internal standard normalizes the sample to sample variations rooted from the experimental workflow.

The protein sample can further comprise an exogenous dystrophin reference protein. In certain aspects the exogenous dystrophin reference protein is added during sample preparation prior to protein isolation. In a particular embodiment the exogenous dystrophin reference protein is added into the sample homogenate prior to precipitation of the proteins in the sample. In some aspects of the invention, the exogenous dystrophin reference protein is added to the protein sample after homogenization and lysis of the biological sample and prior to isolating the protein component of the biological sample.

In certain aspects, the exogenous dystrophin reference protein is metabolically labeled. The metabolically labeled exogenous dystrophin reference protein can be labeled with ¹³C(6)-leucine. The exogenous dystrophin reference protein can share at least 95% amino acid sequence identity to the engineered or endogenous dystrophin protein The exogenous dystrophin reference protein can share at least 95% amino acid sequence identity to the engineered dystrophin protein. The amino acid sequence of the exogenous dystrophin reference protein may be identical to the amino acid sequence of the engineered dystrophin protein. The exogenous dystrophin reference protein may have an amino acid sequence that is at least 95% identical to one or more of SEQ ID NO:242, SEQ ID NO:243, SEQ ID NO:244, or SEQ ID NO:245. The exogenous dystrophin reference protein may have an amino acid sequence that is at least 95% identical to one or more of SEQ ID NO:243, SEQ ID NO:244, or SEQ ID NO:245. The exogenous dystrophin reference protein may have an amino acid sequence that is identical to one or more of SEQ ID NO:242, SEQ ID NO:243, SEQ ID NO:244, or SEQ ID NO:245. The exogenous dystrophin reference protein may have an amino acid sequence that is identical to one or more of SEQ ID NO:243, SEQ ID NO:244, or SEQ ID NO:245. In certain aspects, the exogenous dystrophin reference protein has the amino acid sequence of SEQ ID NO:243.

In some aspects, the exogenous reference peptide is metabolically labeled. The exogenous dystrophin reference peptide may be labeled with ¹³C(6)-leucine.

In some aspects, an exogenous dystrophin reference protein can be proteolyzed to generate or form exogenous dystrophin reference peptides. In certain aspects, an exogenous dystrophin reference peptide is 95% identical to the amino acid sequence LEMPSSLMLEVPTHR (SEQ ID NO:236). In some aspects, the exogenous dystrophin reference peptide has the amino acid sequence of LEMPSSLMLEVPTHR (SEQ ID NO:236). In some aspects, the exogenous dystrophin reference peptide has a sequence of LLQVAVEDR (SEQ ID NO:200). In some aspects, the exogenous dystrophin reference peptide has a sequence of SLEGSDDAVLLQR (SEQ ID NO:179).

In some aspects, the exogenous dystrophin reference peptide may comprise a contiguous amino acid sequence that is present in the exogenous dystrophin protein and the engineered dystrophin protein. In some aspects, the exogenous dystrophin reference peptide may comprise a contiguous amino acid sequence that is present in the exogenous dystrophin protein and is not contiguously present in the endogenous dystrophin protein. The exogenous dystrophin reference peptide may comprise an amino acid sequence of selected from the group consisting of SEQ ID NO:235, SEQ ID NO:236, SEQ ID NO:237, SEQ ID NO:238, SEQ ID NO:239, SEQ ID NO:240, and SEQ ID NO:241.

In some aspects, the exogenous dystrophin reference peptide comprises or consists of the amino acid sequence of WVLLQDQPDLAPGLTTIGASPTQTVTLVTQPVVTK (SEQ ID NO:235). In some aspects, the exogenous dystrophin reference peptide comprises or consists of the amino acid sequence of LEMPSSLMLEVPTHR (SEQ ID NO:236). In some aspects, the exogenous dystrophin reference peptide comprises or consists of the amino acid sequence of MGYLPVQTVLEGDNMET (SEQ ID NO:237). In some aspects, the exogenous dystrophin reference peptide comprises or consists of the amino acid sequence of QSNLHSYVPSTYLTEITHVSQALLEVEQLLNAPDLCAK (SEQ ID NO:238). In some aspects, the exogenous dystrophin reference peptide comprises or consists of the amino acid sequence of LEEQSDQWK (SEQ ID NO:239). In some aspects, the exogenous dystrophin reference peptide comprises or consists of the amino acid sequence of MGYLPVQTVLEGDNMETDTM (SEQ ID NO:240). In some aspects, the exogenous dystrophin reference peptide comprises or consists of the amino acid sequence of LQELTLER (SEQ ID NO:241).

The methods described herein can include (i) isolating the protein component of a biological sample from a subject forming a protein sample; (ii) contacting the protein sample with a first protease forming a protein digest; (iii) applying the protein digest to a dystrophin peptide affinity reagent and eluting affinity selected or reagent bound dystrophin peptides forming a dystrophin peptide enriched sample; and (iv) performing liquid-chromatography-mass spectrometry (LC/MS) on the dystrophin peptide enriched sample. The methods can also include obtaining and processing the sample to form a protein sample. In certain aspects, a biological sample can be homogenized. In certain aspects, a biological sample can be homogenized in a lysis buffer.

Accordingly, in some aspects, the invention provides for a method for measuring the amount of dystrophin in a protein sample isolated from a biological sample comprising: (i) contacting the protein sample with a first protease forming a protein digest; (ii) applying the protein digest to a dystrophin peptide affinity reagent and eluting the dystrophin peptides bound to the affinity reagent forming a dystrophin peptide enriched sample containing endogenous dystrophin peptides, engineered dystrophin peptides, or both; and (iii) performing liquid-chromatography-mass spectrometry (LC/MS) on the dystrophin peptide enriched sample.

Immunoaffinity (IA) Linked LC-MS/MS

Immunoaffinity (IA) linked LC-MS/MS is a quantitative tool that can easily be adapted to enhance sensitivity, specificity, accuracy, and precision for specific protein biomarkers. Initially reported for the quantification of human plasma proteins, peptide IA LC-MS/MS approaches have been developed for several tissue proteins. Embodiments described herein are directed to IA-LC-MS/MS methods that can accurately quantify endogenous dystrophin protein and engineered dystrophin protein (e.g., mini-dystrophin (SEQ ID NO:243)) in skeletal muscle biopsies and be utilized in translational studies in preclinical species and clinical samples without need for modification or new reagents.

IA LC-MS/MS accurately quantified endogenous dystrophin protein and/or engineered dystrophin protein in both human and preclinical species with sufficient sensitivity. Application of this assay in both preclinical and clinical gene therapy studies can serve to validate efficacy results and accelerate regulatory approval.

Assessing Dystrophin in a Tissue Sample

Once a subject has been treated or a suitable control subject or tissue identified, a tissue sample from the treated subject or control can be obtained and various analysis performed on these samples. In certain instances, a piece of tissue from the designated muscle needs to be removed. The most common method for removing a small tissue sample is called a needle biopsy. For this procedure, a medical professional will insert a thin needle through the skin to remove muscle tissue. Depending on the circumstances, the medical professional will use a certain type of biopsy, such as a core needle biopsy. Typically a subject receives local anesthesia for a needle biopsy and should not feel any pain or discomfort.

If the muscle sample is hard to reach—as may be the case with deep muscles—an open biopsy may be performed. In this case, the medical professional will make a small incision in the skin and remove the muscle tissue via the incision. An open biopsy may be performed with the subject under a general anesthesia.

In certain aspects, the biological sample is a tissue sample or biological fluid sample. In certain aspects, the biological sample is a muscle tissue sample. The muscle tissue sample can be a quadricep or bicep sample. The biological sample can be a biopsy sample, in particular a needle biopsy sample. In other aspects, a biological fluid can be blood or a blood fraction. The tissue sample can be fresh, frozen, fixed or unfixed tissues may be used. Any desired convenient procedure may be used for fixing or embedding the tissue sample, as described and known in the art. Thus any known fixatives or embedding materials may be used. The amount of tissue used as a source for the protein sample can weigh about, at least, or at most 1, 10, 100, 500 μg to 1, 2, 3, 4, 5, 10, 20, 50 mg, including all values and ranges there between. In certain aspects, the biological sample is about 100 μg to about 3 mg. In a further aspect, the biological sample is about 200 μg to about 2 mg. In a further aspect, the biological sample is about 500 μg to about 2 mg. The biological sample can be a pre-clinical model species sample or a human clinical sample. In certain aspects, the biological sample is from a human, mouse, rat, monkey, or dog. In particular embodiments, the biological sample is from a human. The human source of a sample can be a patient diagnosed or suspected of having Duchenne's muscular dystrophy (DMD). In certain aspects, the human patient or subject having DMD has been treated with a gene therapy encoding an engineered dystrophin protein.

Sample Processing. Once the tissue is obtained it can be processed for analysis. Processing may involve one or more steps that comprise physical and/or chemical manipulation of the sample. In some embodiments, a sample is incubated with or exposed to one or more chemical agents or enzymes that operate under conditions to promote the action(s) of the chemical agent or enzyme. Such conditions include, but are not limited to, the appropriate temperature, concentration, pH, ionic concentration, or metal concentration; such conditions are well known to those of ordinary skill in the art. Unless otherwise noted, a sample is processed under the conditions that allow for the chemical agent or enzyme to act. A number of examples are provided below. It is specifically contemplated that one or more examples set forth below may be excluded in an embodiment.

The isolation of proteins from a tissue or cells requires homogenization of the respective starting material. Homogenization of tissue in solution is often performed simultaneously with cell lysis. Tissue homogenization involves lysing the cells to release intracellular contents of interest, such as protein components. There are four typical tissue homogenization techniques: chemical homogenization, freeze-thawing, mechanical homogenization, and ultrasonic homogenization.

Mechanical Homogenization. Encompassing equipment like rotor stators and high pressure homogenizers, mechanical homogenization works by using pressure and/or force(s), instead of heat, to mechanically disrupt cells. This technique has the ability to be easily scaled, it is a quick process that provides uniform/consistent results.

Chemical Homogenization. Most disruption methods use some form of lysis buffer or chemical to provide stability when isolating specific biomolecules. Yet some chemicals can be used alone to effectively homogenize tissues. For example, surfactants and detergents target biological membranes by disrupting the hydrophobic/hydrophilic interface. Chemical homogenization is preferable for small samples, as the materials' cost can become overwhelming for industrial-sized products.

Freeze-Thawing. This technique is frequently employed to disrupt bacterial and mammalian cells. A tissue suspension is first frozen and then thawed at room temperature. Ice crystals that formed during the freezing process contract as the sample thaws, which ruptures the cell's membrane. Although it effectively releases recombinant cytoplasmic proteins, the freeze-thaw process requires multiple cycles and requires significant amounts of time.

Ultrasonic Homogenization. Ultrasonic homogenizers, also known as sonicators, rupture tissues through a combination of cavitation and ultrasonic waves. This technique is ideally matched for suspended cellular/subcellular structures, as well as for shearing DNA. However, because it generates a significant amount of heat, ultrasonic homogenization is appropriate only for tissues and molecules that will not be affected by temperature increase.

In certain aspects, a sample is homogenized in a lysis buffer. A lysis buffer can further contain one or more protease inhibitor. The protease inhibitor can include, but is not limited to one or more of aprotinin, bestatin, E-64, leupeptin, and/or pepstatin A. Two or more protease inhibitors can be provided as a cocktail or protease mixture. In certain aspects, the protease inhibitors can be dissolved in DMSO or other solvent and then introduced into the sample preparation solution.

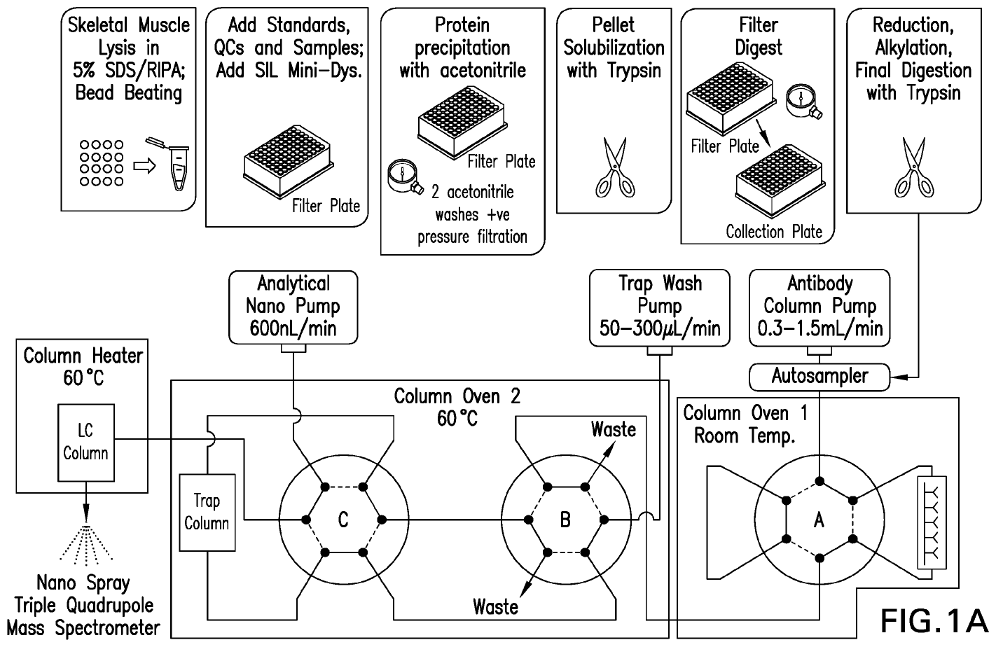

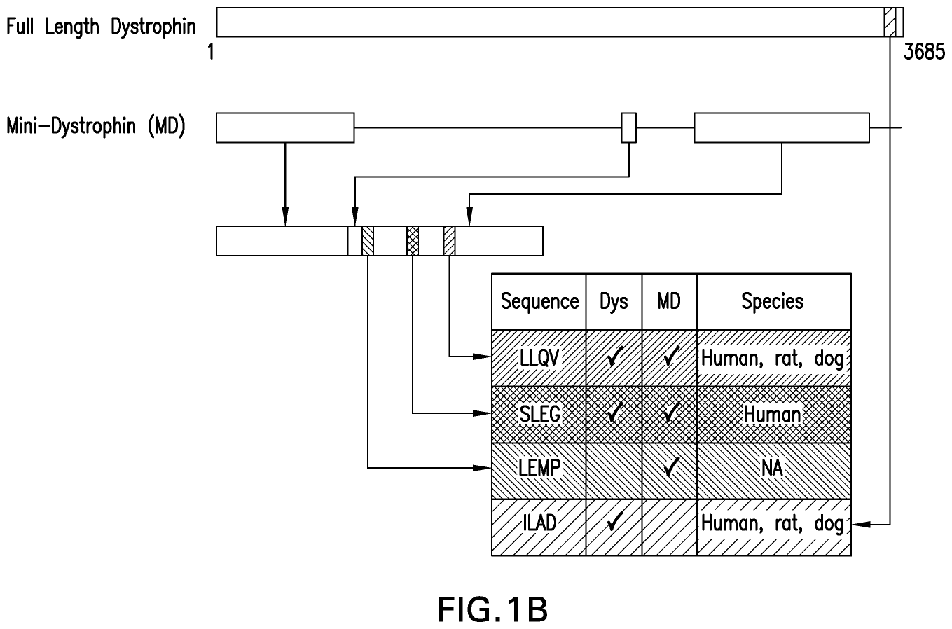

Optimal dystrophin extraction was achieved with 5% SDS in RIPA lysis buffer. SDS is commonly used in Western blot methods, but must be subsequently removed to prevent interference with protein-antibody binding or other downstream steps in an LC-MS assay. Protein precipitation, washing the pellets with acetonitrile, and the peptide enrichment in antibody column coupled online with LC-MS/MS allows for effective removal of SDS as well as optimal cutting temperature compound (OCT), and also served to concentrate the sample (FIG. 1A) (Neubert et al., Bioanalysis. 8, 1551-1555 (2016)). The use of OCT in muscle tissue samples had no effect on assay performance. Sections for LC-MS dystrophin quantification and tissue staining by immunofluorescence for dystrophin localization can therefore be taken from the same tissue block. The ability to analyze adjacent sections from the same tissue block with these two methods may improve the reliability of data interpretation.

In contrast to Western blot methods, the use of a 96-well plate for lysate processing in this assay, with sufficient capacity to include calibrants and quality control (QC) samples for reliable quantification, allowed for a larger number of unknown samples (up to 60) to be prepared simultaneously without the need to run multiple assays. This also enabled the validation study to proceed as discussed below.

In one aspect, tissue sample or skeletal muscle tissue is homogenized in a lysis buffer. In certain aspects the tissue is homogenized using stainless steel beads. In certain aspects, the lysis buffer can be Radio Immuno Precipitation Assay buffer (RIPA), Tissue Extraction Reagent I (TER I), Tissue Extraction Reagent II (TER II), NP40 lysis buffer, Tissue Protein Extraction Reagent (T-PER), Nuclear Protein Extraction Reagent (N-PER), or other appropriate lysis buffer. The lysis buffer can include a detergent. In certain aspects, the detergent concentration in the lysis buffer is, is at least, is at most, about, or between about 0.1, 1, 2, 3, 4, 5, 6, 7, 8, 9, 10, 11, 12, 13, 14 or 15 wt %, including all values and ranges there between. In certain aspects, the detergent is at a concentration of, about, at least, or at most 2, 3, 4, 5, 6, 7, or 8 wt %, including all values and ranges there between. The detergent can be SDS or NP40 or other appropriate detergent. In certain aspects, the lysis buffer comprises 1, 2, 3, 4, 5, 6, 7, 8, 9, or 10% SDS, including all values and ranges there between. In a further aspect, the lysis buffer comprises 3 to 8 wt % SDS. In some aspects, the lysis buffer comprises about 5 wt % SDS.

In certain aspects, the lysis buffer is a Radio Immuno Precipitation Assay (RIPA) buffer. The RIPA buffer can contain about 10 to 50 mM Tris-HCl, 100 to 200 mM NaCl, 0.5 to 1.% NP40, 1 to 2% sodium deoxycholate and 0.1% SDS. In certain aspects, the RIPA buffer contains 25 mM Tris·HCl, 150 mM NaCl, 1% NP40, 0.1 to 1% sodium deoxycholate, 0.1% SDS. In some aspects the lysis buffer contains about 25 mM Tris·HCl, about 150 mM NaCl, about 1% NP40, about 0.1 to about 1% sodium deoxycholate, and about 5% SDS. In some aspects, the RIPA buffer contains 25±0.25 mM Tris·HCl, 150±15 mM NaCl, 1±0.0.01% NP40, about 0.1 to about 1% sodium deoxycholate, and 5±0.5 SDS. In some aspects the lysis buffer contains 25 mM Tris·HCl, 150 mM NaCl, 1% NP40, 0.1 to 1% sodium deoxycholate, and 5% SDS.

The lysis buffer can contain at least one protease inhibitor or a protease inhibitor cocktail. In certain aspects, the lysis buffer contains a plurality of protease inhibitors, i.e., a protease inhibitor cocktail. The protease inhibitor(s) can be present individually and independently at a concentration of, of at least, or of at most about 0.5, 1, to 2 wt %, including all values and ranges there between. In certain aspects, protease inhibitor(s) can be selected from one or more of aprotinin, bestatin, E-64, leupeptin, or pepstatin A.

In certain aspects the tissue sample is homogenized using mechanical homogenization in an appropriate lysis buffer. In certain aspects the lysis buffer comprises a detergent. The lysis buffer can comprise detergent such as SDS, or octylphenoxypolyethoxyethanol (Nonidet™ P40, NP40). In a particular aspect the detergent is SDS. In certain aspects, the lysis buffer can be Tissue Extraction Reagent I (TER I) (Thermo Fisher catalog No. FNN0071-50 mM Tris, pH 7.4, 250 mM NaCl, 5 mM EDTA, 2 mM Na₃VO₄, 1 mM NaF, 20 mM Na₄P₂O₇, 0.02% NaN₃, and a proprietary detergent), Tissue Extraction Reagent II (TER II) (Thermo Fisher catalog No. FNN0081-25 mM Bicine, pH 7.5, 150 mM NaCl, 2 mM Na₃VO₄, 1 mM NaF, 20 mM Na₄P₂O₇, 0.02% NaN₃, and a proprietary detergent.), NP40 lysis buffer (Thermo Fisher catalog No. FNN0021-50 mM Tris, pH 7.4, 250 mM NaCl, 5 mM EDTA, 50 mM NaF, 1 mM Na₃VO₄, 1% Nonidet™ P40 (NP40), 0.02% NaN₃), Tissue-Protein Extraction Reagent (T-PER)(Thermo Fisher catalog No. 78510—a proprietary detergent in 25 mM bicine, 150 mM sodium chloride; pH 7.6), Neuronal-Protein Extraction Reagent (N-PER) (Thermo Fisher catalog No. 87792).

The detergent or detergents can be at a concentration of about, at least about, or at most about 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5 and 15 wt % (or any range derivable therein). In certain aspects, the detergent concentration is about 1 to 10 wt %. In certain aspects there is one detergent that is at a concentration of 5 wt %.

In certain aspects, after homogenization and lysis, two aliquots of the lysate are prepared. One aliquot can be stored for BCA (total protein) analysis. The second aliquot can be used for measuring dystrophin protein. In certain aspects, a SILAC exogenous reference dystrophin protein (SEQ ID NO:243) or other appropriate exogenous dystrophin protein reference is added during sample preparation as an internal standard for the LC-MS/MS assay. The proteins in the sample lysate are separated from the other cell components. In certain aspects, proteins are precipitated onto a filter plate using acetonitrile. The isolated protein sample is digested. In certain aspects the protein sample is digested with Tosyl Phenylalanyl Chloromethyl Ketone (TPCK) treated trypsin. In a further aspect, the sample digest is pulled through the filter plate, and is subsequently reduced, alkylated and subjected to an additional digestion step, a second protease digestion. The digested and treated samples are then immunoaffinity selected. In certain aspects, the digested and treated sample is injected onto a HPLC platform containing an anti-dystrophin peptide antibody column to enrich the tryptic peptides of interest prior to reverse phase nanoflow chromatography. The eluate is from the immunoaffinity step is then analyzed by mass spectrometry. In certain aspects, the eluate reverse phase nanoflow chromatography is introduced into a triple quadrupole mass spectrometer via a nanoflow source interface, operating in multiple reaction monitoring (MRM) mode. The resulting chromatographic peak areas can be generated by TraceFinder General Quan 4.1 or similar software. The results can then exported, for example, to the Watson Laboratory Information Management System (LIMS) using the TraceFinder Digital Gateway Interface.

In certain embodiments a tissue sample is obtained from a subject. At least a portion of the tissue sample is homogenized and the cellular proteins isolated. The cellular protein isolate is digested into peptide fragments. Dystrophin peptides are selected for using immunoaffinity. The selected dystrophin peptides are then analyzed and quantitated using liquid chromatography/mass spectrometry (LC/MS).

Protein isolation. Sample preparation depends on the origin of the cells or the tissue. In some aspects, the first step is usually homogenization or sonication followed by protein precipitation and solubilization in a suitable buffer. In some aspects of the invention, the homogenized biological sample is treated with an organic solvent so as to precipitate proteins, forming a protein precipitate. The protein precipitate may be isolated as a filter retentate, thereby forming the protein sample. Organic solvents such as chloroform, methanol, acetone, acetonitrile, and TCA are commonly used as protein precipitating reagents. In some aspects, the organic solvent is selected from the group consisting of acetic acid, acetone, acetonitrile, chloroform, dimethylformamide, dimethyl sulfoxide, dioxane, ethanol, isopropanol, methanol, 1-propanol, TCA, and tetrahydrofuran, or a combination thereof. In certain aspects the organic solvent is acetone or acetonitrile. The proteins can be precipitated by addition of 1, 2, 3, 4, 5, 6, 7, 8, 9, 10 volumes of solvents to 1 volume of homogenate. 800 to 140 sample. (1 sample to 5.7 solvent) Proteins can be precipitated at a temperature of or about −10, −5, 0, 1, 5, 10, 15, 20, 25 to 30° C., including all values and ranges there between. In certain aspects the proteins are precipitated at a temperature between about 15° C. and about 25° C. In certain aspects the proteins are precipitated at a temperature between about 18° C. and about 22° C. In particular aspects the proteins are precipitated at about 20° C. After a sufficient time the protein precipitation mixture can be centrifuged or filtered to isolate the protein precipitate. Once isolated and washed the protein precipitate can be solubilized in an appropriate solution.

Protein digestion. Once the proteins have been isolated, the protein sample can be exposed to proteolysis or proteolyzed by using one or more proteases (or proteinases) to liberate or fragment the proteins in the sample into peptides resulting in a peptide solution or peptide sample. Proteases are involved in digesting long protein/polypeptide chains into shorter fragments by splitting the peptide bonds that link amino acid residues. In some aspects of the invention, the first or second protease is selected from the group consisting of serine proteases, threonine proteases, cysteine proteases, aspartate proteases, glutamic acid proteases, and metalloproteases. Protease digestion serves to produce peptide fragments from proteins/polypeptides to facilitate analysis by mass spectrometry.

The first or second protease may be at a concentration of about 10 ng/μL to about 1 μg/μL. The first or second protease may be at a concentration of about 10 ng/μL to about 100 ng/μL. The first or second protease may be at a concentration of about 50+/−5 ng/μL. The first or second protease may be at a concentration of about 50 ng/μL.

In some aspects of the invention, the first or second protease is trypsin- or a lysine-specific proteinase. These enzymes have the advantages of being reliable, specific and produce a suitable number of peptides. Other suitable proteases induce digestions at aspartate or glutamate residues, and include endoproteinase Glu-C or endoproteinase Asp-N. Chymotrypsin may also be used. Proteinases of broad specificity may generate many peptides, and the peptides may be very short.

In certain aspects, the first or second protease is trypsin, and in particular aspects, it is Tosyl Phenylalanyl Chloromethyl Ketone (TPCK) treated trypsin. Trypsin is a serine protease. It cleaves proteins into peptides with an average size of 700-1500 daltons. Trypsin is highly specific, cutting at the carboxyl side of arginine and lysine residues. The C-terminal arginine and lysine peptides are charged, making them detectable by MS. Trypsin is highly active and tolerant of many additives. Alternatively the first or second protease may be selected from the group consisting of chymotrypsin, pepsin, LysC, LysN, AspN, GluC and ArgC, and can be used for protein digestion prior to mass spectrometry.

In certain aspects a first digestion can be performed with a first protease followed by a second digestion with a second protease, which may be the same or a different protease. In some aspects of the invention, the first protease is the same as the second protease.

In some aspects of the invention, the second protease is selected from the group consisting of serine proteases, threonine proteases, cysteine proteases, aspartate proteases, glutamic acid proteases, and metalloproteases. In certain aspects, the second protease is trypsin, and in particular aspects, it is Tosyl Phenylalanyl Chloromethyl Ketone (TPCK) treated trypsin. Alternatively the second protease may be selected from the group consisting of chymotrypsin, pepsin, LysC, LysN, AspN, GluC and ArgC, and can be used for protein digestion prior to mass spectrometry.

The second protease may be at a concentration of about 10 ng/μL to about 1 μg/μL. The second protease may be at a concentration of about 10 ng/μL to about 100 ng/μL. The second protease may be at a concentration of about 50 ng/μL. The second protease may be at a concentration of about 50 ng/μL.

In some aspects of the invention, the first or second protease may be provided in a protease buffer, and the protease buffer may comprise at least a chaotropic agent, organic solvent and buffer salts. The protease buffer may comprise urea, acetonitrile and phosphate buffered saline. The protease buffer may comprise urea at a concentration from about 0.05 M to about 4 M. The protease buffer may comprise acetonitrile at a concentration from about 1% to about 25%. The protease buffer may comprise about 0.8M urea and about 10% acetonitrile in phosphate buffered saline. The protease buffer may comprise 0.8M urea and 10% acetonitrile in phosphate buffered saline.

In some embodiments, the protein mixture is heated during formation of the digested mixture. Heating increases the rate at which digestion occurs, thereby decreasing the amount of time necessary for a sufficient time to pass for the protein mixture to form a digested mixture. The protein mixture can be heated to a temperature above room temperature. For example, in some embodiments, the protein mixture is heated to a temperature from about 35, 40, 45, 50, 55, to about 60° C. for a time from about 1, 2, 3, 4, 5, 6, 7, 8, 9, 10, 11, to about 12 hours. The heating should be sufficient to accelerate digestion, without destroying the protease or polypeptides. A variety of ways can be used to heat the digested mixture. For example, the digested mixture can be heated by placing the tissue bearing the protein mixture into an oven or water bath. The protein mixture can be held within an air-tight container during heating in order to prevent drying. The digestion can be repeated 2, 3, 4, or more times. Typically the proteolytic enzyme(s) is at a concentration of about, at least about, or at most about 10, 20, 30, 40, 50, 60, 70, 80 90, 100, 200, 300, 400, 500, 600, 700, 800, 900 ng/μL to 1 μg/μL (or any range derivable therein). In certain aspects the proteolytic enzyme(s) is at a concentration of about 50 ng/μL.

The peptide solution can be treated with agents to stabilize or maintain the integrity of the peptides. In certain aspects the peptides can be treated with a reducing agent so as to denature disulfide bonds. The reducing agent may be selected from the group consisting of dithiothreitol (DTT), 2-mercaptoethanol (2-ME), or Tris(2-carboxyethyl) phosphine hydrochloride (TCEP). In certain aspects the reducing agent is DTT. The reducing agent can be used at a concentration of about, at least about, or at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mM or any range derivable therein. In certain aspects the reducing agent is at a concentration of about 50 mM to about 250 mM in the peptide sample. In certain aspects the reducing agent is at a concentration of about 150+/−15 mM. In certain aspects the reducing agent is at a concentration of about 135 mM to 165 mM in the peptide sample. In certain aspects the reducing agent is at a concentration of about 150 mM.

The peptide solution can also be treated with an alkylating agent to minimize reformation of disulfide bonds. The alkylation agent may be selected from the group consisting of Iodoacetamide (IAA), methyl methanethiosulfonate, and N-ethylmaleimide. In certain aspects the alkylation agent is IAA. The alkylating agent can be at a concentration of about, at least about, or at most about 20, 50, 100, 150, 200, 250, 300, 350, 400, 450 or 500 mM (or any range derivable therein). In certain aspects the alkylating agent is at a concentration of about 20 mM to about 500 mM in the peptide sample. In certain aspects the alkylating agent is at a concentration of about 200 mM to about 400 mM in the peptide sample. In certain aspects the alkylating agent is at a concentration of about 300±30 mM in the peptide sample. In certain aspects the alkylating agent is at a concentration of about 270 mM to about 330 mM in the peptide sample. In certain aspects the alkylating agent is at a concentration of about 300 mM.

Peptide Enrichment. Once a peptide sample is obtained, affinity chromatography (e.g., immunoaffinity chromatography) can be used to isolate the dystrophin peptides present in the protein digestion. In some aspects of the invention the method comprises more than one dystrophin peptide affinity reagent, such that two or more different dystrophin peptides can be captured from the peptide sample.

In some aspects of the invention, the bound dystrophin peptides are from an endogenous dystrophin protein, an engineered dystrophin protein, and/or an exogenous dystrophin reference protein. The dystrophin peptide affinity reagent may be coupled to a column matrix forming a dystrophin peptide affinity column. The dystrophin peptide affinity column may be capable of specifically binding 1, 2, or more dystrophin peptides the dystrophin peptide affinity column binds a common dystrophin peptide that is present in both the endogenous dystrohin protein and the engineered dystrophin protein. The dystrophin peptide affinity column may bind an engineered dystrophin specific peptide that is present in the engineered dystrophin protein and not present in the endogenous dystrophin protein. The dystrophin peptide affinity reagent may comprise an anti-dystrophin peptide antibody. The anti-dystrophin peptide antibody may be raised against an endogenous dystrophin peptide. The anti-dystrophin peptide antibody may be raised against an engineered dystrophin peptide.

This technique utilizes high specificity polyclonal antibodies. The term “polyclonal antibodies” refers to a population of antibody polypeptides that contain multiple species of antigen binding sites capable of interacting with a particular antigen, e.g., a dystrophin peptide. Polyclonal antibodies are antibodies made by injecting animals with protein or peptide antigens, and then after a secondary immune response is stimulated, isolating antibodies from whole serum. Thus, polyclonal antibodies are a heterogeneous mix of antibodies that recognize several epitopes of the antigen injected into the animals. In certain aspects, polyclonal antibodies are rabbit polyclonal antibodies. Polyclonal antibodies are typically a purified fraction of antibodies obtained from the blood of an immunized animal. Usually the antigen is applied intravenously, intradermally, intramuscularly, or subcutaneously to the animal, preferably together with an adjuvant which triggers the formation of antibodies. Frequently the application of the antigen occurs three to four times whereby the time difference between each application (booster) of the antigen is 2-6 weeks. When the antibody titer has reached the desired level a large amount of blood is taken from the animal. The serum is obtained from the blood and subsequently the antibodies are separated from the serum. This can be done with suitable separation means which allow the enrichment of the antibodies (e.g., suitable columns).

Such highly specific molecules are extremely valuable tools for rapid, selective purification of antigens or peptides. In principle, the antibody is coupled (immobilized) on a column support and this is used to selectively adsorb antigen from a mixture containing many other antigens. The antigens for which the antibody has no affinity can be washed away, and the purified antigen then eluted from the bound antibody with an elution buffer.

In some aspects, the anti-dystrophin peptide antibody is raised against a peptide comprising or consisting of an amino acid sequence selected from the group consisting of SEQ ID NO:179, SEQ ID NO:200, SEQ ID NO:235, SEQ ID NO:236, SEQ ID NO:237, SEQ ID NO:238, SEQ ID NO:239, SEQ ID NO:240, or SEQ ID NO:241. The anti-dystrophin peptide antibody may be raised against a peptide comprising or consisting of the amino acid sequence of SEQ ID NO:235. The anti-dystrophin peptide antibody may be raised against a peptide comprising or consisting of the amino acid sequence of SEQ ID NO:236. The anti-dystrophin peptide antibody may be raised against a peptide comprising or consisting of the amino acid sequence of SEQ ID NO:237. The anti-dystrophin peptide antibody may be raised against a peptide comprising or consisting of the amino acid sequence of SEQ ID NO:238. The anti-dystrophin peptide antibody may be raised against a peptide comprising or consisting of the amino acid sequence of SEQ ID NO:239. The anti-dystrophin peptide antibody may be raised against a peptide comprising or consisting of the amino acid sequence of SEQ ID NO:240. The anti-dystrophin peptide antibody may be raised against a peptide comprising or consisting of the amino acid sequence of SEQ ID NO:241.

The anti-dystrophin peptide antibody may be raised against a common dystrophin peptide. The anti-dystrophin peptide antibody may be raised against a peptide comprising or consisting of the amino acid sequence of LLQVAVEDR (SEQ ID NO:200). The anti-dystrophin peptide antibody may be raised against a peptide comprising or consisting of the amino acid sequence of SLEGSDDAVLLQR (SEQ ID NO:179).

The anti-dystrophin peptide antibody may be a polyclonal antibody. The anti-dystrophin peptide antibody may be a monoclonal antibody.

In some aspects, the invention relates to an anti-dystrophin peptide antibody. In some aspects, the invention relates to methods of generating anti-dystrophin antibodies. In some aspects, the invention relates to methods of using anti-dystrophin antibodies to measure, quantify, or assess dystrophin in a biological sample.

In some aspects, the invention relates to a peptide comprising or consisting of the amino acid sequence selected from the group consisting of SEQ ID NO:235, SEQ ID NO:236, SEQ ID NO:237, SEQ ID NO:238, SEQ ID NO:239, SEQ ID NO:240, or SEQ ID NO:241. In some aspects, the invention relates to methods of generating peptides of the invention. In some aspects, the invention relates to methods of using peptides of the invention to measure, quantify, or assess dystrophin in a biological sample.

The peptide solution resulting from one or more protein digestion can be used a source for isolation of proteolytic fragments or peptides of the dystrophin protein, i.e., dystrophin peptides. In certain embodiments dystrophin peptides can be selected by contacting a peptide solution with a dystrophin peptide affinity reagent, such as an affinity column comprising a dystrophin peptide specific antibody. The dystrophin specific affinity agent can specifically bind to one or more peptides of dystrophin. The dystrophin peptide can be an endogenous dystrophin peptide, an engineered dystrophin peptide or both an endogenous dystrophin peptide and engineered dystrophin peptide. The affinity column can have the capability of binding 1, 2, 3, 4, 5, 6, 7, 8, 9, 10 or more peptides identified in any of SEQ ID NO:1 to 241. The dystrophin peptide enriched sample may comprise endogenous dystrophin peptides. The dystrophin peptide enriched sample may comprise engineered dystrophin peptides. The dystrophin peptide enriched sample may comprise exogenous dystrophin reference peptides—said exogenous dystrophin reference peptides being produced by the proteolytic digestion by the first and/or second protease of an exogenous dystrophin reference protein added to the biological sample, or added to the protein sample.

An “epitope” refers to the area or region of an antigen to which an antibody specifically binds, e.g., an area or region comprising residues that interacts with the antibody. Epitopes can be linear or conformational.

An antibody or antibodies that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular antigen than it does with alternative antigens. An antibody “specifically binds” or “preferentially binds” to a target if it binds with greater affinity, avidity, more readily, and/or with greater duration than it binds to other substances. It is also understood by reading this definition that, for example, an antibody (or moiety or epitope) which specifically or preferentially binds to a first target may or may not specifically or preferentially bind to a second target. As such, “specific binding” or “preferential binding” does not necessarily require (although it can include) exclusive binding.

Peptide Analysis

Once dystrophin peptides are selected from the peptide sample, the peptides will be further analyze using fractionation and mass spectrometry. In certain aspects, the process of determining the quantity of dystrophin protein expressed by the cells in the sample includes comparing a mass ratio of particular peptides to a calibration curve, wherein the calibration curve is a mathematical relationship between a known amount of the peptide and the quotient of the known amount of peptide and a constant amount of a standard peptide.

Sample Fractionation. The selected peptides can be further physically separated or isolated prior to undergoing ionization. The peptide sample can be fractionated using liquid chromatography upstream of the ionization component of the mass spectrometer. In certain aspect the chromatography step is a reverse phase nanoflow chromatography. The some aspects, the method further comprises a reversed phase nano liquid chromatography step. The liquid chromatography step is coupled with mass spectrometry of the resulting fractions. In some aspects, the reverse phase nanoflow chromatography column is a C18 column having 3 μm particle size, 100 Å pore size, and dimensions of 75 μm×15 cm.

Mass spectrometry. A “mass spectrometer” is an analytical instrument that can be used to determine the molecular weights of various substances, such as proteins and nucleic acids. It can also be used in some applications, e.g., to determine the sequence of protein molecules and the chemical composition of virtually any material. Typically, a mass spectrometer comprises four parts: a sample inlet, an ionization source, a mass analyzer, and a detector. A sample is optionally introduced via various types of inlets, e.g., solid probe, GC, or LC, in gas, liquid, or solid phase. The sample is then typically ionized in the ionization source to form one or more ions. The resulting ions are introduced into and manipulated by the mass analyzer. Surviving ions are detected based on mass to charge ratio. In one embodiment, the mass spectrometer bombards the substance under investigation with an electron beam and quantitatively records the result as a spectrum of positive and negative ion fragments. Separation of the ion fragments is on the basis of mass to charge ratio of the ions. If all the ions are singly charged, this separation is essentially based on mass. Traditional quantitative MS has used electrospray ionization (ESI) followed by tandem MS (MS/MS) while newer quantitative methods are being developed using matrix assisted laser desorption/ionization (MALDI) followed by time of flight (TOF) MS. In some aspects, the mass spectrometer is a triple quadrupole mass spectrometer configured for multiple reaction monitoring (MRM). In some aspects, the method further comprises using LCMS, wherein the mass spectrometry includes electrospray ionization of the dystrophin peptide fractions and detection of ionized peptide fragments. In some aspects, the method further comprises using triple quadrupole mass spectrometer in multiple reaction monitoring (MRM) mode to detect ionized forms of one or more selected from the group consisting of engineered dystrophin peptides, exogenous dystrophin reference peptides, and endogenous dystrophin peptides. In some aspects, analyzing the dystrophin peptide fraction further comprises quantitating the dystrophin peptides detecting using a peptide calibration curve.

Electrospray Ionization (ESI)

ESI is a convenient ionization technique developed by Fenn and colleagues (Fenn et al., Science, 246(4926):64-71, 1989) that is used to produce gaseous ions from highly polar, mostly nonvolatile biomolecules, including lipids. The sample is injected as a liquid at low flow rates (1−10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice. Kabarle et al., Anal. Chem. 65(20):972A-986A (1993). A potential difference of between 1 to 5 kV (but more typically 2 to 3 kV) is applied to the capillary by power supply to generate a high electrostatic field (106 to 107 V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multicharged gas phase ions in the form of an ion beam. The ions are then collected by the grounded (or negatively charged) interface plate and led through an orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; 6,756,586, 5,572,023 and 5,986,258.

ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., Anal. Chem., 74:2446, 2000) and bioactive peptides (Desiderio et al., Biopolymers, 40:257, 1996). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide. Bucknall et al., J. Am. Soc. Mass Spectrometry, 13(9):1015-27 (2002). Protein quantification has been achieved by quantifying tryptic peptides. Mirgorodskaya et al., Rapid Commun. Mass Spectrom., 14:1226, 2000. Complex mixtures such as crude extracts can be analyzed, but in some instances sample cleanup is required. Gobom et al., Anal. Chem. 72:3320, 2000. Desorption electrospray is a new associated technique for sample surface analysis.

SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

LD-MS and LDLPMS

Laser desorption mass spectroscopy (LD-MS) involves the use of a pulsed laser, which induces desorption of sample material from a sample site-effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and the separation of fragments is due to different size causing different velocity. Each ion mass will thus have a different flight-time to the detector.

One can either form positive ions or negative ions for analysis. Positive ions are made from regular direct photoionization, but negative ion formation requires a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of negative ion spectra.

MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers, peptide and protein analysis (Zaluzec et al., Protein Expr. Purif., 6:109, 1995; Roepstorff et al., EXS, 88:81, 2000), DNA and oligonucleotide sequencing, and the characterization of recombinant proteins. Recently, applications of MALDI-TOF-MS have been extended to include the direct analysis of biological tissues and single cell organisms with the aim of characterizing endogenous peptide and protein constituents. Li et al., Trends Biotechnol., 18:151 (2000); Caprioli et al., Anal. Chem., 69:4751 (1997).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of polypeptides (i.e., peptides and proteins) is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use. Wang et al., J. Agric. Food. Chem., 48:3330 (2000); Desiderio et al., Biopolymers, 40:257 (1996). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Mass analyzers separate the ions according to their mass-to-charge ratio. There are a variety of analyzers that can be used, including sector instruments, time-of-flight, quadrupole mass filter, three dimensional quadrupole ion trap, cylindrical ion trap, etc.

Sector instruments. A sector field mass analyzer uses a static electric and/or magnetic field to affect the path and/or velocity of the charged particles in some way.

Time-of-flight. The time-of-flight (TOF) analyzer uses an electric field to accelerate the ions through the same potential, and then measures the time they take to reach the detector. If the particles all have the same charge, their kinetic energies will be identical, and their velocities will depend only on their masses. Ions with a lower mass will reach the detector first.

Quadrupole mass filter. Quadrupole mass analyzers use oscillating electrical fields to selectively stabilize or destabilize the paths of ions passing through a radio frequency (RF) quadrupole field created between 4 parallel rods. Only the ions in a certain range of mass/charge ratio are passed through the system at any time, but changes to the potentials on the rods allow a wide range of m/z values to be swept rapidly, either continuously or in a succession of discrete hops.

Ion traps. The quadrupole ion trap works on the same physical principles as the quadrupole mass analyzer, but the ions are trapped and sequentially ejected. The cylindrical ion trap mass spectrometer (CIT) is a derivative of the quadrupole ion trap where the electrodes are formed from flat rings rather than hyperbolic shaped electrodes.

Kits

Some embodiments relate to immunoassay kits for measuring, quantifying, or assessing dystrophin proteins in a biological sample. The kits can comprise one or more antibody composition that bind specifically to a particular dystrophin peptide. In certain aspects the antibody composition is provided in the form of a column matrix or a pre-packed affinity column. The kit can further include a homogenization reagents and buffers, proteolytic reagents and buffers, affinity chromatography buffers, reference proteins and/or peptides, and the like. By way of example, such kits can be useful for detecting or quantitating the levels of dystrophin in a tissue sample.

### Definitions

The term “dystrophin protein” refers to all variants and derivative transcribed and translated from a natural or recombinant dystrophin gene or nucleic acid.

The term “dystrophin peptide” refers to peptides or protein fragments that are present in or result from proteolysis of a dystrophin protein.

The term “endogenous” gene or protein as used herein, refers to proteins produce by transcription and translation of genes originating from the unmodified genome of an organism, tissue, or cell, i.e., the naturally occurring, non-manipulated gene/protein of an organism, tissue, or cell.

As used herein, the term “engineered” refers to any genetic material and/or resulting protein that is not encoded or expressed by the unmodified genome of an organism, tissue, or cell. Such “engineered” proteins can thus exclude any protein expressed from genetic material normally found within a unmanipulated cell. An engineered protein includes, in some embodiments, any protein expressed or caused to be expressed from recombinant genetic material introduced into a cell. Additionally, “engineered” includes proteins generated via an expression vector/cassette or other expression vehicle that is distinct from the cellular genome, but includes material integrated into the genome at non-natural location(s) or engineered nucleic acid that replaces defective genomic sequences. In some embodiments, recombinant genetic material is extrachromosomal, while in others, all or part of it becomes integrated into a cell's chromosome (intrachromosomal or transgenic).

The term “endogenous dystrophin protein” as used herein, refers to dystrophin proteins produce by transcription and translation of the dystrophin gene in an unmodified genome of an organism, tissue, or cell, i.e., the naturally occurring, non-manipulated dystrophin gene/protein of an organism, tissue, or cell.

The term “engineered dystrophin protein” refers to any dystrophin encoding genetic material and/or resulting dystrophin protein that is not encoded or expressed by an unmodified genome of an organism, tissue, or cell. Such “engineered” dystrophin proteins can thus exclude any protein expressed from genetic material normally found within a unmanipulated cell. An engineered dystrophin protein includes any dystrophin protein expressed or caused to be expressed from recombinant genetic material introduced into a cell.

The term “common dystrophin peptide” refers to a peptide that is present in both an endogenous dystrophin protein and an engineered dystrophin protein.

The term “endogenous dystrophin peptide” refers to amino segments or fragments that are present in an endogenous dystrophin protein.

The term “engineered dystrophin specific peptide” refers to amino segments or fragments that are only present in an engineered dystrophin protein.

The term “exogenous dystrophin reference protein” refers to a dystrophin protein that is synthesized independently of the biological sample.

The term “exogenous dystrophin reference peptide” refers to amino segments or fragments that are present in an exogenous dystrophin reference protein.

The term “variant” refers to peptides or polypeptides of the differing at one or more amino acid residues of a reference molecule, such as dystrophin proteins and peptides described herein.

An antibody “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarity determining regions (CDRs), and contribute to the formation of the antigen-binding site of antibodies.

“Framework” (FR) residues are antibody variable domain residues other than the CDR residues. A VH or VL domain framework comprises four framework sub-regions, FR1, FR2, FR3 and FR4, interspersed with CDRs in the following structure: FR1-CDR1-FR2-CDR2-FR3-CDR3-FR4.

An “epitope” refers to the area or region of an antigen to which an antibody specifically binds, e.g., an area or region comprising residues that interacts with the antibody. Epitopes can be linear or conformational.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. It is also understood by reading this definition that, for example, an antibody (or moiety or epitope) which specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “biological sample” means sample material from a living organism. The term “biological sample” is intended to include tissues, cells, and biological fluids isolated from a subject, as well as tissues, cells and fluids present within a subject. Biological samples of the present technology include, e.g., but are not limited to, whole blood, plasma, and muscle. Biological samples can be obtained from biopsies of internal organs or from tissues.

“About” or “approximately,” when used in connection with a measurable numerical variable, refers to the indicated value of the variable and to all values of the variable that are within the experimental error of the indicated value (e.g. within the 95% confidence interval for the mean) or within 10 percent of the indicated value, whichever is greater. Numeric ranges are inclusive of the numbers defining the range.

As used herein, “vector” means a nucleic acid construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “identity,” as known in the art, refers to a relationship between the sequences of two or more polypeptide molecules or two or more nucleic acid molecules, as determined by comparing the sequences. In the art, “identity” also means the degree of sequence relatedness between polypeptide or nucleic acid molecule sequences, as the case may be, as determined by the match between strings of nucleotide or amino acid sequences. “Identity” measures the percent of identical matches between two or more sequences with gap alignments addressed by a particular mathematical model of computer programs (i.e. “algorithms”).

The term “similarity” is a related concept, but in contrast to “identity”, refers to a measure of similarity which includes both identical matches and conservative substitution matches. Since conservative substitutions apply to polypeptides and not nucleic acid molecules, similarity only deals with polypeptide sequence comparisons. If two polypeptide sequences have, for example, 10 out of 20 identical amino acids, and the remainder are all nonconservative substitutions, then the percent identity and similarity would both be 50%. If in the same example, there are 5 more positions where there are conservative substitutions, then the percent identity remains 50%, but the percent similarity would be 75% (15 out of 20). Therefore, in cases where there are conservative substitutions, the degree of similarity between two polypeptide sequences will be higher than the percent identity between those two sequences.

### EXAMPLES

Representative examples of methods and materials are described herein, although methods and materials similar or equivalent to those described herein can also be used in the practice or testing of the present invention. The materials, methods, and examples are illustrative only and not intended to be limiting.

### Example 1

### Peptide Selection

Peptide selection and ranking was based on the following criteria. Human full-length endogenous dystrophin amino acid sequence FASTA file from Uniprot (accession number P11532)(SEQ ID NO:242) was used to generate theoretical tryptic endogenous peptides with minimum 6 and maximum 30 residues with in-silico digestion using Pepdigest from EMBOSS. Using BLAST+ 2.9.0 tool from NCBI, each individual endogenous dystrophin peptide was used in a BLASTP search against reference proteome from cynomolgus monkey (Macaca fascicularis), rat (Rattus norvegicus), dog (Canis familiaris), and human specific databases to identify human endogenous dystrophin specific and conserved endogenous dystrophin peptides between human and other species dystrophin. In certain aspects, a single affinity reagent can be used for those peptides conserved across pre-clinical and clinical species. BLASTP parameters were set as the following: −evalue 200000, gapopen 15, gapextend 3, word size 2, matrix PAM30, subject besthit, max hsps 1, max target seqs 1, comp based stats 0. Engineered dystrophin protein sequence was used for sequence alignment of the engineered dystrophin protein sequence with human full length endogenous dystrophin. Engineered dystrophin specific peptides were identified (see Table 11).

Peptides were ranked based on the Peptide Spectrum Matches (PSM) scores reported in publically available proteomics database developed by Technische Universität München (proteomicsdb.com). Peptides were ranked based on predicted antigenicity score ranging from 0 to 5 using an online bioinformatics tool developed by Thermo Fisher Scientific (available from thermofisher.com).

### Example 2

### Sample Preparation and Assessment

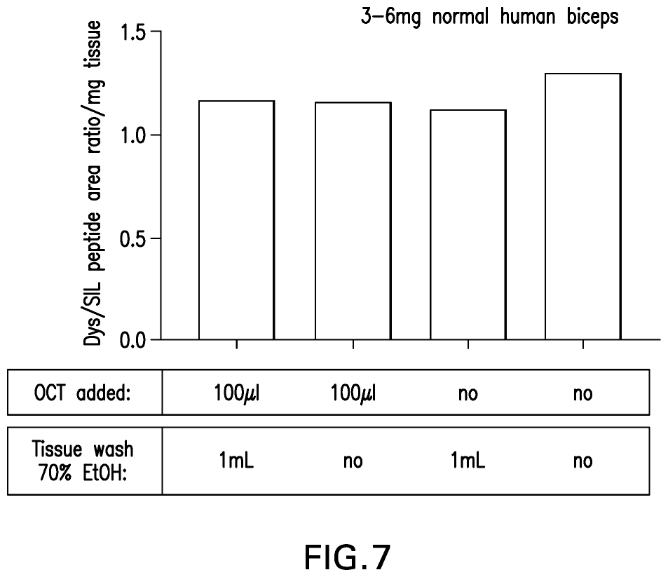

In this study tolerance to optimal cutting temperature (OCT) medium was evaluated, and it was shown that up to 15 μL OCT per mg tissue is tolerated by the assay and there is no need to wash the OCT prior to the sample preparation (FIG. 7). Excessive amounts of OCT need to be washed out with cold 70% ethanol prior to sample preparation.

Method workflow is summarized in FIG. 1A. Twenty patient samples each from healthy, BMD, and DMD muscle were received as cryosections (10 slices of 10 μm) in a moderate amount of optimal cutting temperature (OCT) medium. Snap-frozen samples or samples with excessive OCT had 1 mL of ice cold 70% ethanol added and were vortexed 5×5 seconds (medium speed). Samples with excessive OCT were subsequently centrifuged at 14,000 rpm at 4° C. for 10 min and the supernatant discarded and air dried at ambient temperature for 5 min. All samples were weighed prior to tissue lysis.

### Example 3

### SDS Titration

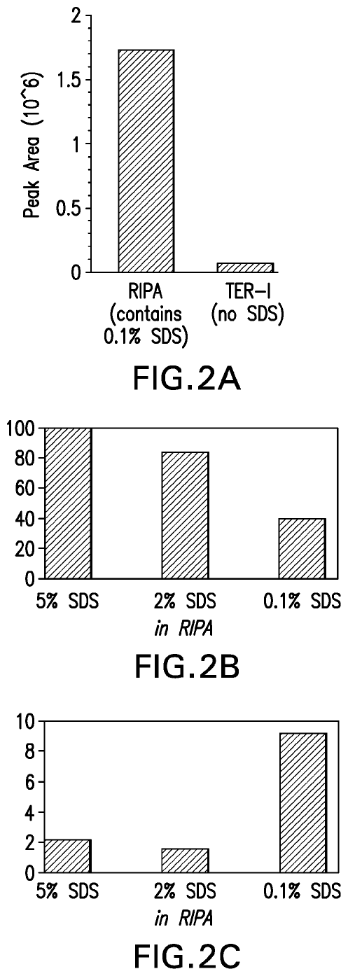

The effect of sodium dodecyl sulfate (SDS) in the lysis buffer on percentage dystrophin protein extraction was evaluated by comparing Radio Immuno Precipitation Assay (RIPA) buffer (0.1% SDS) with TER-I (no SDS) in an experiment and RIPA buffer with no added SDS, 5% SDS and 10% SDS content in another experiment. Data showed that presence of presence of SDS in RIPA buffer leads to significant increase in extraction of dystrophin protein from human skeletal muscle tissue. Also further titration of SDS showed that modified RIPA with 5% SDS leads to maximum extraction of dystrophin protein from human skeletal muscle tissue (FIGS. 2A-2C).

### Example 4

### Tissue Lysis and Extraction

Approximately 10 beads of a 0.9-2.0-mm blend of stainless steel beads (NextAdvance™ Averill Park, NY) were added to 500 μL lysis buffer consisting of RIPA buffer with 5% SDS (Fisher Scientific International, Hampton, NH) and 1×HALT Protease Inhibitor Cocktail (ThermoFisher Scientific, Waltham, MA) at 100 μL per 10 mL lysis buffer. Tissues were homogenized at room temperature using the Bullet Blender® (Next Advance) for −10 min. Lysates were clarified by centrifugation at room temperature and 14,000 rpm for 20 min and a 20 μL aliquot of each sample retained for the bicinchoninic assay (BCA) protein determination assay.

Healthy and DMD muscle lysates were diluted at a ratio of 1.5-mg tissue mass to 1000 μL lysis buffer, with the volume of lysis buffer adjusted to the mass of the sample cut. A 20 μL aliquot of each sample was stored at room temperature in a clean Eppendorf tube (Hauppauge, NY) until the total protein assay could be performed. Calibration curves and quality control samples were prepared by adding a 120 μL aliquot to the appropriate well on the filter plate.

Stable isotope-labeling by amino acids in cell culture (SILAC) engineered dystrophin (an exogenous dystrophin reference protein) was prepared by diluting 200 μL of the 20,000 fmol/mL stock with 1800 μL of surrogate matrix. The surrogate matrix was prepared by adding serum to the lysis buffer at a final concentration of 0.7%. SILAC exogenous engineered dystrophin reference protein was added to each sample (20 μL of 2,000 fmol/mL) and incubated for 4-6 min at room temperature.

### Example 5

### Protein Precipitation

A 120 μL aliquot of each sample was precipitated in 800 μL acetone at room temperature for 50-70 min with gentle mixing at 300 rpm in the ThermoMixer® (Eppendorf). The supernatant was removed using positive-pressure manifold connected to nitrogen gas and the flow through discarded. Protein pellets were washed in 1 mL acetone per sample at room temperature and the wash filtered out by positive pressure manifold. This step was repeated and the filter dried. Protein pellets were solubilized in 120 μL of 50 ng/μl tosyl phenylalanyl chloromethyl ketone (TPCK)-treated trypsin per sample, the filter plate sealed and incubated at 37° C. in the ThermoMixer at 900 rpm for 12-18 h.

In a further optimization of the method during the method validation, it was shown that using acetonitrile (ACN) for protein precipitation resulted in more efficient and reproducible recovery of dystrophin from the skeletal muscle tissue lysates in comparison to acetone. This comparison was made with the spiked-in SILAC peptides recovered from the lysates precipitated with acetone vs ACN. In this alternative method, a 120 μL aliquot of each sample was precipitated in 800 μL acetonitrile at room temperature for 25-30 min with gentle mixing at 300 rpm in the Fisher Isotemp Shake Touch. The supernatant was removed using positive-pressure manifold connected to nitrogen gas and the flow through discarded (pressure ˜20 psi). Protein pellets were washed in 1 mL acetonitrile per sample at room temperature and the wash filtered out by positive pressure manifold. This step was repeated and the filter air dried. Protein pellets were solubilized in 120 μL of buffer comprised of 80% PBS, 10% 8M Urea, 10% Acetonitrile and 50 μg/mL of tosyl phenylalanyl chloromethyl ketone (TPCK)-treated trypsin (6.0 μg per well). The filter plate sealed and incubated at 37° C. in the Fisher Isotemp Shake Touch at 900 rpm for 12-18 h.

### Example 6

### On-Filter Protein Digestion and Positive Pressure Filtration

A filter plate was used for the sample throughput improvement and advancing the workflow capacity in comparison to the use of the Eppendorf tubes for handling of the tissue lysates. Comparison between protein precipitation and pellet digestion in Eppendorf tubes and filter plates showed similar efficiency of dystrophin recovery from the skeletal muscle lysates indicating the benefit of filter plates use in the assay. In the same experiment also showed that filter plate handling of protein precipitation in room temperature result in similar recovery of dystrophin peptides with filter plate protein precipitation done at 4° C.

The plate was briefly spun down (5400 rpm for 15 s) and pellets further solubilized by adding 30 μL of 50 ng/μL TPCK-trypsin in PBS to each sample (1.5 μg per well), incubating in the ThermoMixer at 37° C. with shaking at 900 rpm for 2.5-3.5 h. The plate was briefly spun down and using positive-pressure manifold and the digested mixture was filtered directly into a 1.5 mL 96-deep well collection plate placed below the filter plate. An initial pressure of 20 psi was used and adjusted accordingly. The filter plate was washed using 100 μL (50 μL may also be used) of PBS and positive-pressure filtration. Disulfide reduction was carried out at 60° C. for 50-70 min by adding 10 μL of freshly prepared 150 mM dithiothreitol to each sample, followed by alkylation at room temperature, in the dark, for 50-70 min with 10 μL of 300 mM iodoacetamide. Samples were subsequently digested at 37° C. for 23 h by adding 10 μL of 100 ng/μL LysC-trypsin. Samples were injected onto a high-performance LC-MS (Dionex UltiMate™ 3000; ThermoFisher).

### Example 7

### IA LC-MS Assay

A detailed description of IA LC-MS has been described by Palandra et al (2013) Anal Chem 85(11):5522-5529.). The online immunoaffinity (IA), liquid chromatography (LC) method configuration was optimized to trap column forward elution and back wash for more robustness and reproducibility. IA-LC-MS/MS configuration is summarized in FIG. 1A (see also Table 1). The antibody column is maintained at room temperature. Briefly, the sample is loaded and flow through the antibody column is controlled by valve A. Peptides of interest are captured and unwanted peptides and contaminants are removed to waste (valve B). The antibody column is washed with 25 mM ammonium formate, followed by 0.5% trifluoroacetic acid in water to elute target peptides onto the C18 trap. Chromatographic separation is subsequently achieved in the Thermo Easy Spray PepMap C18 at a flow rate of 0.6 μl/min with acetonitrile/formic acid/water buffers. For this assay, the temperature controlled autosampler was set to 5° C. and an injection volume of 80 μL. Analysis of human samples used a single anti-peptide Ab column holding two anti-peptide antibodies against LLQVAVEDR (SEQ ID NO:200) and LEMPSSLMLEVPTHR (SEQ ID NO:236) peptides. Analysis of the DMD^{mdx} rat samples utilized anti-peptide antibodies against peptides LLQVAVEDR (SEQ ID NO:200) and SLEGSDDAVLLQR (SEQ ID NO:179).

All custom anti-peptide antibodies (immunoglobulin G) were generated by Cambridge Research Biochemicals and the anti-peptide column prepared in-house. The anti-peptide antibody columns were prepared using IDEX Biosafe Column System (2.1 mm×30 mm×2.0 μm). The antibody solution was prepared so as to include anti-LLQVAVEDR (SEQ ID NO:200) and anti-LEMPSSLMLEVPTHR (SEQ ID NO:236) antibodies ranging from 0.30-0.40 (for anti-LLQV) and 0.8-1.0 (for anti-SEQ ID NO:236) mg per antibody per column. Anti-SLEGSDDAVLLQR (SEQ ID NO:179) was used at 0.5 mg per antibody column. The trap column and nano LC were maintained at a temperature of 60° C. and eluent from the anti-antibody columns was collected on a p-Precolumn Cartridge fitted with a PepMap™ 100 C18 (ThermoFisher) with 5-μm particle size, 100 Å pore size, 300 μm diameter, 5-mm length. Chromatographic separation was achieved using Easy-Spray PepMap C18 column (75 μm×15 cm).

The eluate from nanoflow chromatography was introduced into Easy Spray Ionization Source (ThermoFisher) at 60° C. with a coupling spray voltage of 3000 V and a collision gas pressure of 1.5 mTorr. Detection of peptides was performed on a Quantiva Triple Quadrupole MS (ThermoFisher) by multiple reaction monitoring (MRM) in positive ion mode. Transition summing was used to enhance the signal and sensitivity of the assay for each peptide, including calibration standards and quality controls. Major precursor ions to fragment transitions were scanned multiple times during each MRM cycle. Transitions, including multiple product ions, were then combined which generated a summed, quantifiable area under the curve (AUC). MS acquisition time was −14.5 min, with expected retention times of 11.1±1.5 and 12.1±1.5 min for LLQVAVEDR (SEQ ID NO:200) and LEMPSSLMLEVPTHR (SEQ ID NO:236), respectively.

A high flow rate at the antibody column pump and the large binding capacity of the antibody columns allowed a relatively large volume of the processed sample to be loaded rapidly while taking advantage of the sensitivity gains provided by the analytical nanoflow chromatography and nanospray ionization on the mass spectrometer. This is important for the detection of low abundance proteins, such as dystrophin. Bound peptides are eluted from the antibody column and captured on a trap column. The LC flow path was configured such that peptides were then forward eluted through the trap onto the analytical column while any build-up within the system was back-flushed into waste (valve B), ensuring that the trap remains clean.

Mass spectrometry parameter setting and multiple reaction monitoring (MRM) transitions are provide in Table 2. Echo transition summing can be easily applied to improve common dystrophin peptide (SEQ ID NO:200) by increasing peak area and averaging random noise. Echo transition summing was also used for engineered dystrophin peptide (SEQ ID NO:236) to further enhance MS sensitivity and improve signal-to-noise.

TABLE 1
Liquid chromatography system description.
Micro Pump:
Mobile Phases and Program
A: 25 mM Ammonium Formate
B: 0.50% Trifluoroacetic Acid in Water
C: 0.75% Formic Acid, 3% Isopropanol in Water

| Retention | Flow rate | | | |
|---|---|---|---|---|
| Time (min) | (mL/min) | % A | % B | % C |
| 0.00 | 0.600 | 100 | 0 | 0 |
| 3.00 | 0.600 | 100 | 0 | 0 |
| 3.00 | 0.300 | 0 | 100 | 0 |
| 6.00 | 0.300 | 0 | 100 | 0 |
| 6.00 | 1.00 | 0 | 0 | 100 |
| 9.50 | 1.00 | 0 | 0 | 100 |
| 9.50 | 1.00 | 100 | 0 | 0 |
| 12.00 | 1.00 | 100 | 0 | 0 |
| 12.50 | 0.100 | 100 | 0 | 0 |

Loading Pump:
Mobile Phases and Program
A: 0.1% Trifluoroacetic Acid in Water
C: 90/10/0.1% Acetonitrile/Water/Formic Acid

| Retention | Flow rate | | |
|---|---|---|---|
| Time (min) | (mL/min) | % A | % C |
| 0.00 | 0.250 | 15 | 85 |
| 0.05 | 0.250 | 15 | 85 |
| 1.00 | 0.250 | 100 | 0 |
| 2.75 | 0.250 | 100 | 0 |
| 3.00 | 0.050 | 100 | 0 |
| 3.25 | 0.050 | 70 | 30 |
| 9.00 | 0.050 | 70 | 30 |
| 12.10 | 0.050 | 70 | 30 |
| 12.15 | 0.250 | 70 | 30 |
| 12.50 | 0.250 | 15 | 85 |

Nano (NC) Pump:
Mobile Phases and Program
A: 98/2/0.1% Water/Acetonitrile/Formic Acid
B: 90/10/0.1% Acetonitrile/Water/Formic Acid

| Retention | Flow rate | | |
|---|---|---|---|
| Time (min) | (μL/min) | % A | % B |
| 0.00 | 0.600 | 15 | 85 |
| 1.00 | 0.600 | 15 | 85 |
| 2.00 | 0.600 | 98 | 2 |
| 6.00 | 0.600 | 98 | 2 |
| 11.00 | 0.600 | 65 | 35 |
| 11.50 | 0.600 | 65 | 35 |
| 11.50 | 0.600 | 15 | 85 |

Valve Switching
(Column Oven NCS-3500RSC)

| Retention | | |
|---|---|---|
| Time (min) | Valve 2 | Valve 2 |
| Initial | 1_2 | 6_1 |
| 3.25 | 6_1 | 6_1 |
| 6.00 | 1_2 | 1_2 |
| 12.00 | 1_2 | 6_1 |

Valve Switching
(Column Oven TCC-3200RS)

| Retention Time (min) | Valve 1 |
|---|---|
| Initial | 6_1 |

TABLE 2
Mass spectrometer parameters (A) and MRM transitions (B). (H) indicates labelled
exogenous reference peptide.
A)

| MS system | Thermo Quantiva Triple Quadrupole |
|---|---|
| Ion source | Thermo Fisher Scientific Easy Spray Ionization Source |
| LC coupling spray | 3000 |
| voltage, V | |
| Heated capillary | 300 |
| temperature, ° C. | |
| Collision gas | 1.5 |
| pressure, mTorr | |
| MS acquisition | 14.5 |
| time, min | |
| Polarity | Positive |
| Data type | Centroid |
| Q1, FWHM | 0.70 |
| Q3, FWHM | 0.70 |
| Scan width, m/z | 0.010 |

B)

| | Start | End | | | | Frament | Collision | Dwell |
|---|---|---|---|---|---|---|---|---|
| Compound | Time | Time | | Precursor | Product | Type/ | Energy | Time |
| SEQ ID NO | (min) | (min) | Polarity | (m/z) | (m/z) | Number | (V) | (ms) |
| 200 | 8 | 11.6 | Positive | 521.798 | 589.294 | [y5] | 19.4 | 60 |
| 200 | 8 | 11.6 | Positive | 521.798 | 688.362 | [y6] | 19.4 | 60 |
| 200 | 8 | 11.6 | Positive | 521.798 | 816.421 | [y7] | 19.4 | 60 |
| 200 (H) | 8 | 11.6 | Positive | 527.818 | 589.294 | [y5] | 19.5 | 60 |
| 200 (H) | 8 | 11.6 | Positive | 527.818 | 688.362 | [y6] | 19.5 | 60 |
| 200 (H) | 8 | 11.6 | Positive | 527.818 | 816.421 | [y7] | 19.5 | 60 |
| 236 | 11.6 | 14.5 | Positive | 580.632 | 609.335 | [y5] | 26.3 | 20 |
| 236 | 11.6 | 14.5 | Positive | 580.632 | 609.339 | [y5] | 26.3 | 20 |
| 236 | 11.6 | 14.5 | Positive | 580.632 | 609.343 | [y5] | 26.3 | 20 |
| 236 | 11.6 | 14.5 | Positive | 580.632 | 609.347 | [y5] | 26.3 | 20 |
| 236 | 11.6 | 14.5 | Positive | 580.632 | 609.351 | [y5] | 26.3 | 20 |
| 236 | 11.6 | 14.5 | Positive | 580.632 | 609.355 | [y5] | 26.3 | 20 |
| 236 | 11.6 | 14.5 | Positive | 580.632 | 609.359 | [y5] | 26.3 | 20 |
| 236 | 11.6 | 14.5 | Positive | 580.632 | 738.385 | [y6] | 26.3 | 20 |
| 236 | 11.6 | 14.5 | Positive | 580.632 | 738.389 | [y6] | 26.3 | 20 |
| 236 | 11.6 | 14.5 | Positive | 580.632 | 738.393 | [y6] | 26.3 | 20 |
| 236 (H) | 11.6 | 14.5 | Positive | 586.652 | 609.337 | [y5] | 26.3 | 20 |
| 236 (H) | 11.6 | 14.5 | Positive | 586.652 | 609.341 | [y5] | 26.3 | 20 |
| 236 (H) | 11.6 | 14.5 | Positive | 586.652 | 609.345 | [y5] | 26.3 | 20 |
| 236 (H) | 11.6 | 14.5 | Positive | 586.652 | 609.349 | [y5] | 26.3 | 20 |
| 236 (H) | 11.6 | 14.5 | Positive | 586.652 | 609.353 | [y5] | 26.3 | 20 |
| 236 (H) | 11.6 | 14.5 | Positive | 586.652 | 609.357 | [y5] | 26.3 | 20 |
| 236 (H) | 11.6 | 14.5 | Positive | 586.652 | 609.361 | [y5] | 26.3 | 20 |
| 236 (H) | 11.6 | 14.5 | Positive | 586.652 | 738.387 | [y6] | 26.3 | 20 |
| 236 (H) | 11.6 | 14.5 | Positive | 586.652 | 738.391 | [y6] | 26.3 | 20 |
| 236 (H) | 11.6 | 14.5 | Positive | 586.652 | 738.395 | [y6] | 26.3 | 20 |

For this assay, the temperature controlled autosampler was set to 5° C. and an injection volume of 100 μL. An anti-antibody column maintained at room temperature was used and all anti-peptide antibodies packed at 0.5 mg each. All custom anti-peptide antibodies (immunoglobulin G) were generated by Cambridge Research Biochemicals and the anti-peptide column prepared in-house. Anti-peptide antibody columns for LEMPSSLMLEVPTHR (SEQ ID NO:236) and LLQVAVEDR (SEQ ID NO:200) were prepared using Applied Biosystems column body or equivalent (2.1 mm×30 mm). The trap column and nano LC were maintained at a temperature of 60° C. and eluent from the anti-antibody columns was collected on a p-Precolumn Cartridge fitted with a PepMap™ 100 C18 (ThermoFisher) with 5 μm particle size, 100 Å pore size, 300 μm diameter, 5 mm length. Chromatographic separation was achieved using Easy-Spray PepMap C18 column (3 μm particle size, 100 Å pore size 75 μm×15 cm).

The eluate from nanoflow chromatography was introduced into Easy Spray Ionization Source (ThermoFisher) at 60° C. with a coupling spray voltage of 3000 V and a collision gas pressure of 1.5 mTorr. Detection of peptides was performed on a Quantiva Triple Quadrupole MS (ThermoFisher) by multiple reaction monitoring (MRM) in positive ion mode. Transition summing was used to enhance the signal and sensitivity of the assay for each peptide, including calibration standards and quality controls. Major precursor ions to fragment transitions were scanned multiple times during each MRM cycle. Transitions, including multiple product ions, were then combined which generated a summed, quantifiable area under the curve (AUC). MS acquisition time was ˜14.5 min.

Peptides SEQ ID NO:200 and SEQ ID NO:236 were successfully validated for the analysis of dystrophin and mini-dystrophin in human skeletal muscle sample lysates for the concentration range 20.0-3333 fmol/mL (=pM) for both peptides. Inter- and intra-assay precision were ≤20% (≤25% at the lower limit of quantification, LLQVAVEDR (SEQ ID NO:200)) and inter- and intra-run relative error was within ±20% (±25% at LLOQ) (Table 3). The normal lysate pool was successfully assigned both an endogenous dystrophin concentration of 1470 fmol/mL and a total protein concentration of 0.490 mg/mL determined by BCA assay.

TABLE 3
Summary of inter- and intra-assay accuracy and precision
based on peptides LLQV and LEMP from five accuracy
and precision runs during validation

| Peptide | Precision, (% CV) | | Accuracy (% RE) | |
|---|---|---|---|---|
| SEQ ID | Intra-assay | Inter-assay | Intra-assay | Inter-assay |
| 200 | 3.7 to 23.4 | 7.8 to 17.9 | −10.0 to 9.0 | −5.0 to 8.5 |
| 236 | 2.7 to 22.5 | 9.1 to 20.6 | −12.8 to 18.2 | −8.0 to 12.5 |

Intra- and inter-run % CV was ≤20% (except ≤25% at QCLOQ) and intra and inter-run % RE was within ±20% (except ±25% at QCLOQ).
CV, coefficient of variation;
QCLOQ, limit of quantitation quality control sample;
RE, relative error.

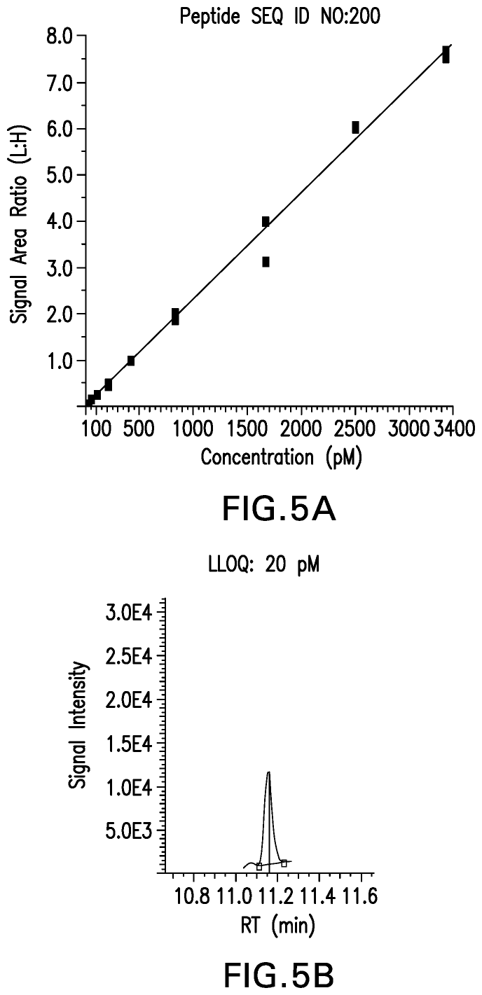

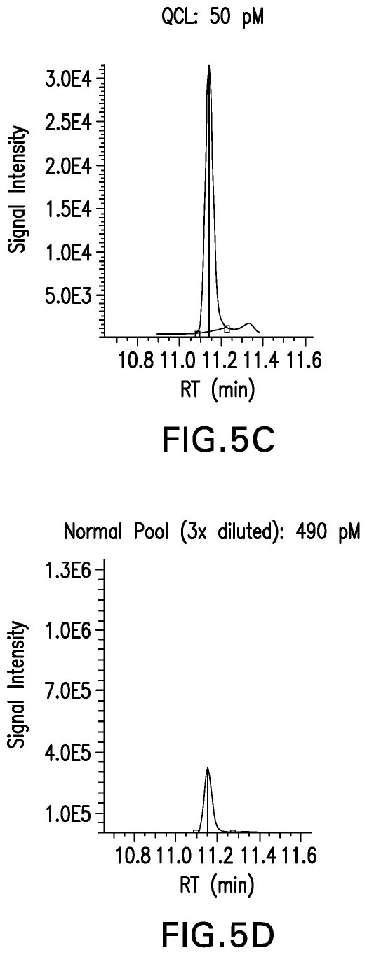

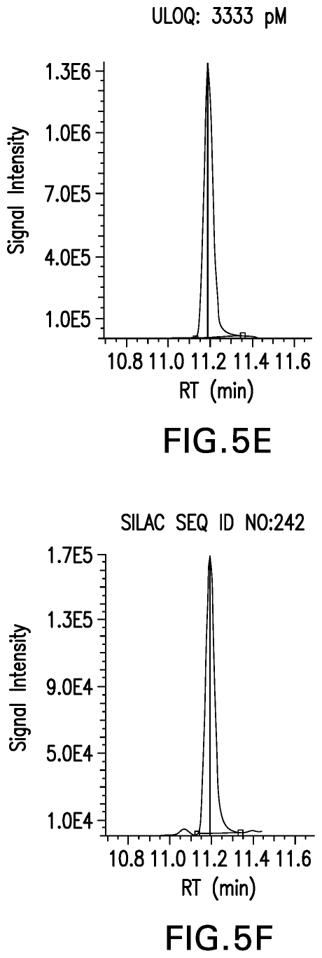

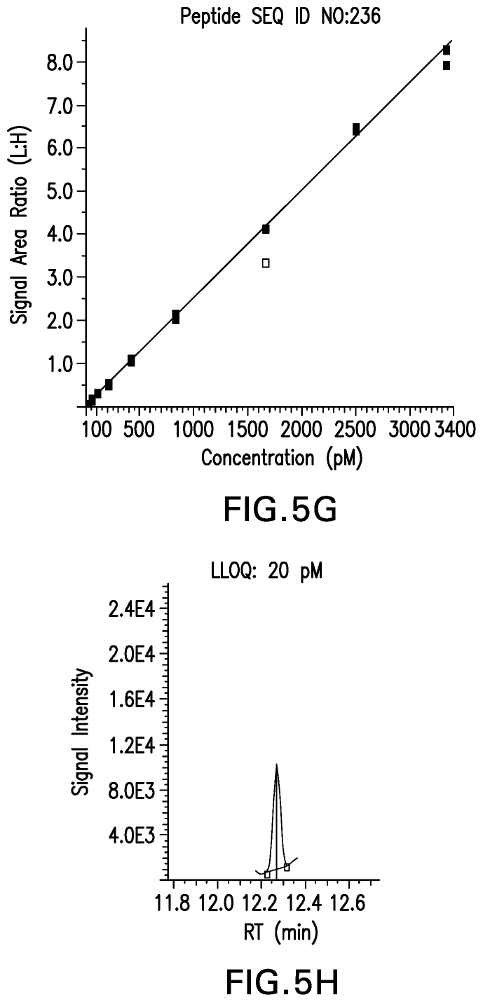

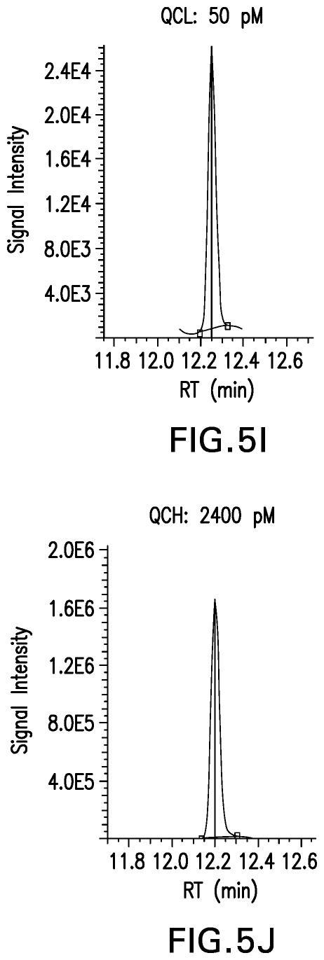

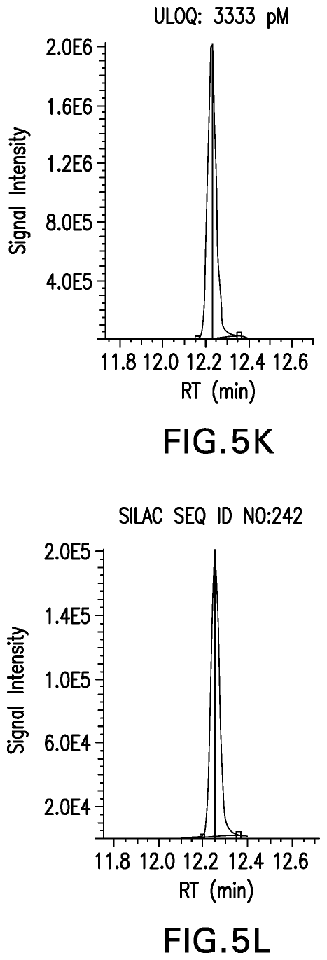

Back-calculated mini-dystrophin calibration standards for LLQV and LEMP peptides are presented in Table 4. Calibration curves and representative ion chromatograms for peptides LLQVAVEDR (SEQ ID NO:200) and LEMPSSLMLEVPTHR (SEQ ID NO:236) are shown in FIGS. 5A-5L.

Processed sample stability was established for up to one week at −70° C. (Table 5) and autosampler stability established for up to 72 h. The normal lysate was found to be stable over a 30-week testing period following storage at −70° C. (Table 6) and up to two cycles of freezing and thawing (Table 7). Assay sensitivity was maintained, confirming the stability of the calibrator. These results confirm a high performance of the method, particularly in terms of relative error and precision even for low-level detection of endogenous dystrophin and engineered dystrophin. The reproducibility of the method compares favorably with that of other published techniques for the quantification of dystrophin, including capillary Western immunoassays (PLoS One 13(4):e0195850) and high-throughput immunofluorescence (PLoS One 13(3):e0194540), and exceeds that reported for traditional Western blots (Neurology 83(22):2062-2069).

TABLE 4
Calibration curve performance for A) LLQV and B) LEMP peptides.

| | | | | | | | | | |
|---|---|---|---|---|---|---|---|---|---|
| CS 1 | CS 2 | CS 3 | CS 4 | CS 5 | CS 6 | CS 7 | CS 8 | CS 9 | CS 10 |
| 20 | 26 | 52.1 | 104 | 208 | 417 | 833 | 1670 | 2500 | 3300 |
| fmol/mL | fmol/mL | fmol/mL | fmol/mL | fmol/mL | fmol/mL | fmol/mL | fmol/mL | fmol/mL | fmol/mL |

A)

| | | | | | | | | | | |
|---|---|---|---|---|---|---|---|---|---|---|
| Mean | 19.5 | 26.5 | 55.4 | 108 | 214 | 408 | 827 | 1580 | 2460 | 3210 |
| SD | 1.13 | 2.3 | 3.4 | 11.3 | 15.6 | 27.0 | 55.0 | 160 | 200 | 256 |
| CV, % | 5.8 | 8.6 | 6.1 | 10.5 | 7.3 | 6.6 | 6.7 | 10.2 | 8.1 | 8.0 |
| RE, % | −2.5 | 1.9 | 6.3 | 3.8 | 2.9 | −2.2 | −0.7 | −5.4 | −1.6 | −2.7 |

B)

| | | | | | | | | | | |
|---|---|---|---|---|---|---|---|---|---|---|
| Mean | 19.8 | 25.4 | 56.2 | 104 | 217 | 412 | 816 | 1590 | 2470 | 3250 |
| SD | 0.44 | 2.1 | 3.8 | 10.1 | 16.4 | 31.7 | 62.8 | 154 | 208 | 315 |
| CV, % | 2.2 | 8.2 | 6.7 | 9.8 | 7.6 | 7.7 | 7.7 | 9.6 | 8.4 | 9.7 |
| RE, % | −1.0 | −2.3 | 7.9 | 0.0 | 4.3 | −1.2 | −2.0 | −4.8 | −1.2 | −1.5 |

Summary table presents calibration standards above 20 fmol/mL from initial validation runs (that extended down to 7 fmol/mL) and supplemental validation runs with lowest calibration standard at 20 fmol/mL. Calibrants below 20 fmol/mL not shown.

TABLE 5
One-week freezer stability (−70° C.) of endogenous
dystrophin and mini-dystrophin in skeletal muscle
lysate for A) LLQV and B) LEMP.

| | | | |
|---|---|---|---|
| QCL 20.0 | QCM1 200 | QCM2 800 | QCH 2400 |
| fmol/mL | fmol/mL | fmol/mL | fmol/mL |

A)

| | | | | |
|---|---|---|---|---|
| | 16.7 | 203 | 841 | 2630 |
| | 16.8 | 125^{a} | 822 | 2130 |
| | 17.0 | 146^{a} | 820 | 2620 |
| | 16.9 | 173 | 736 | 2430 |
| | 9.98^{a} | 193 | 570^{a} | 2250 |
| | 17.2 | 148^{a} | 757 | 2370 |
| Mean | 15.8 | 165 | 758 | 2410 |
| SD | 2.84 | 30.1 | 101 | 199 |
| % CV | 18.0 | 18.2 | 13.3 | 8.3 |
| % RE | −21.0^{b} | −17.5 | −5.3 | 0.4 |
| n | 6 | 6 | 6 | 6 |

B)

| | | | | |
|---|---|---|---|---|
| | 22.3 | 209 | 843 | 2620 |
| | 19.0 | 132^{a} | 844 | 2150 |
| | 16.6 | 154^{a} | 814 | 2610 |
| | 18.8 | 176 | 744 | 2390 |
| | 14.2^{a} | 195 | 564^{a} | 2220 |
| | 16.8 | 158^{a} | 767 | 2430 |
| Mean | 18.0 | 171 | 763 | 2400 |
| SD | 2.76 | 28.4 | 105 | 194 |
| % CV | 15.3 | 16.6 | 13.8 | 8.1 |
| % RE | −10.0 | −14.5 | −4.6 | 0.0 |
| n | 6 | 6 | 6 | 6 |

Note:
Initial QC range displayed
CV: coefficient of variation; n: number of samples; QCH: High Quality Control; QCL: Low Quality Control;
QCM1: Medium Quality Control 1; QCM2: Medium Quality Control 2.; RE: relative error; SD: standard deviation
^{a}RE >20%
^{b}RE >25% (applies only to LLOQ)

TABLE 6
Normal lysate stability over a 30-week testing
period following storage at −70° C.

| | Test Week 7 | Test Week 26 | Test Week 30 | |
|---|---|---|---|---|
| | Concentration | Concentration | Concentration | |
| | 1470 fmol/mL | 1470 fmol/mL | 1470 fmol/mL | Overall |
| | 1270 | 1330 | 1510 | — |
| | 1550 | 1330 | 1450 | — |
| | 1380 | 1460 | 1450 | — |
| | 1360 | 1500 | 1400 | — |
| | 1320 | 1510 | 1020 | — |
| | 1450 | 1490 | 1510 | — |
| Mean | 1390 | 1430 | 1390 | 1400 |
| SD | 99.5 | 92.4 | 186 | 127 |
| % CV | 7.2 | 6.5 | 13.4 | 9.0 |
| % RE | −5.4 | −2.7 | −5.4 | −6.0 |
| n | 6 | 6 | 6 | 18 |

Stability is demonstrated if the mean concentration of the stored QC samples is within 20% compared to their nominal concentration with a CV ≤20%.

TABLE 7
Stability of mini-dystrophin in skeletal muscle lysate after cycles of freezing and
thawing for A) LLQV and B) LEMP.

| | | | | | |
|---|---|---|---|---|---|
| FT1-QCL | FT2-QCL | FT3-QCL | FT1-QCH | FT2-QCH | FT3-QCH |
| 20.0 fmol/mL | 20.0 fmol/mL | 20.0 fmol/mL | 2400 fmol/mL | 2400 fmol/mL | 2400 fmol/mL |

A)

| | | | | | | |
|---|---|---|---|---|---|---|
| | 79.5^{b} | 17.8 | 16.5 | 2410 | 2360 | 2300 |
| | 23.3 | 17.3 | 7.55^{a} | 2230 | 2340 | 2380 |
| | 18.1 | 16.9 | 13.0^{a} | 2120 | 1990 | 2240 |
| | 16.8 | 13.2^{a} | 18.0 | 2380 | 2320 | 2340 |
| | 14.1^{a} | 17.3 | 90.3^{b} | 2530 | 2640 | 2510 |
| | 16.6 | 17.1 | 15.9^{a} | 2580 | 2270 | 2490 |
| Mean | 17.8 | 16.6 | 14.2 | 2380 | 2320 | 2380 |
| SD | 3.41 | 1.69 | 4.13 | 175 | 208 | 106 |
| % CV | 19.2 | 10.2 | 29.1 | 7.4 | 9.0 | 4.5 |
| % RE | −11.0 | −17.0 | −29.0 | −0.8 | −3.3 | −0.8 |
| n | 5 | 6 | 5 | 6 | 6 | 6 |

B)

| | | | | | | |
|---|---|---|---|---|---|---|
| | 67.76 | 15.0^{a} | 15.9 | 2310 | 2170 | 2190 |
| | 20.2 | 17.9 | 13.1^{a} | 2220 | 1930 | 2300 |
| | 21.8 | 17.5 | 16.3 | 1950 | 1890ª | 2150 |
| | 18.8 | 17.7 | 18.7 | 2350 | 2280 | 2370 |
| | 16.5 | 17.5 | 81.1^{b} | 2540 | 2460 | 2550 |
| | 18.8 | 18.6 | 17.4 | 2490 | 2210 | 2330 |
| Mean | 19.2 | 17.4 | 16.3 | 2310 | 2160 | 2320 |
| SD | 1.96 | 1.23 | 2.08 | 212 | 216 | 143 |
| % CV | 10.2 | 7.1 | 12.8 | 9.2 | 10.0 | 6.2 |
| % RE | −4.0 | −13.0 | −18.5 | −3.8 | −10.0 | −3.3 |
| n | 5 | 6 | 5 | 6 | 6 | 6 |

Note:
Freeze-Thaw is acceptable for up to 2 cycles.
CV: coefficient of variation; FT: Freeze/thaw cycle; n: number of samples; QCH: High Quality Control; QCL: Low Quality Control; RE: relative error; SD: standard deviation.
^{a}RE >20%.
^{b}Outlier based on Dixon Test (99% confidence).

### Example 8

### Quantification Strategy

Dystrophin expression was calculated as a percentage of dystrophin expression in the healthy sample pool. Engineered dystrophin protein (fmol/mL) was calculated as a percentage of total protein (mg) and back-calculated against an engineered dystrophin protein standard curve. Calibrant standards were freshly prepared in 0.7% human serum in lysis buffer. Engineered dystrophin protein was spiked into normal or DMD lysate prior to protein extraction and digestion. Engineered dystrophin protein calibrant concentrations were 3333, 2500, 1667, 833, 407, 208, 104, 52.1, 26.0, 20.0, and 0 fmol/mL. Duplicate calibration standards were included in each 96-well plate.

Total protein quality control (QC) samples were freshly prepared and measured in replicates of four. Healthy tissue QC samples were normal tissue lysate 3× diluted in lysis buffer, endogenous normal tissue lysate, and endogenous normal tissue lysate (1500 μg/mL) spiked with 1500 μg/mL BSA. Equivalent DMD tissue QC samples were prepared along with a fourth QC sample, which was prepared by mixing endogenous normal tissue lysate and DMD tissue lysate in a 1:1 ratio. Peptide concentrations from healthy, BMD, and DMD lysate samples were normalized to total protein content, as determined by a photometric BCA assay (Pierce™ BCA Protein Analysis kit, ThermoFisher).

All LC-MS/MS data were generated by TraceFinder™ General Quan v. 4.2 (ThermoFisher). Peaks were identified by retention time using the associated raw data file from a calibration curve sample (single peak detection using the ICIS detection algorithm, default settings). Peaks areas from all associated transition summing were compiled and the AUC imported to Watson LIMS. AUC data was then analyzed for concentration based on back-calculated data from the calibration curve. The calibration curve was fitted with a linear regression model and 1/x² weighting.

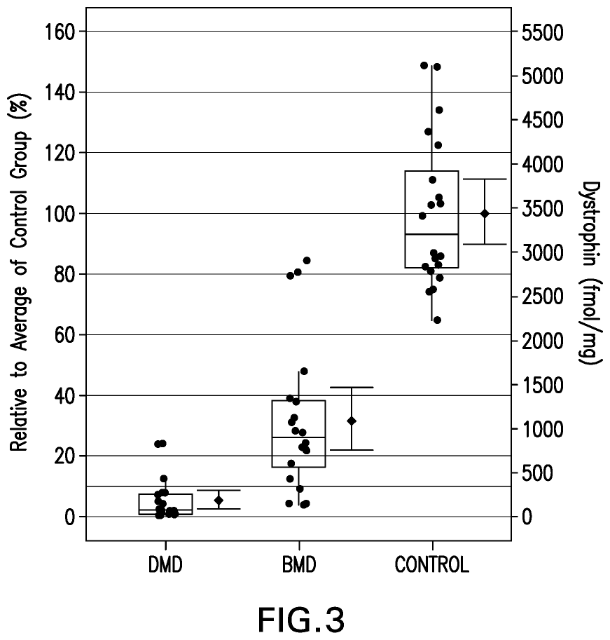

Control-tissue dystrophin expression had an expression range 65-149% of the control mean expression (100%, median 93%; 3440 fmol/mg) based on the LLQV peptide (FIG. 3). In BMD muscle, the expression range was 4-85% of normal mean (mean 32%, median 26%; 1089 fmol/mg) and in DMD muscle, the expression range was 0.4-24.1% of normal mean (mean 5%, median 2%; 186 fmol/mg), with no dystrophin quantifiable in 7 of 20 DMD samples. The LLOQ of the LC-MS assay for dystrophin in the tissue lysate was 20.0 fmol/mL; however, the normalized tissue LLOQ of dystrophin in fmol/mg of total protein depends on the amount of tissue or total protein used and is derived by dividing the lysate dystrophin LLOQ (20.0 fmol/mL) by the total protein concentration in lysate (mg/mL). For all below limit of quantitation (BLQ) results, dystrophin lysate concentrations were imputed as 0.5*LLOQ for the summary statistics and graphical presentation. One patient who was diagnosed with DMD had an in-frame mutation (ex 3-30 del), generally characteristic of BMD, with a 24.1% expression level. Importantly, there was no overlap in mean (95% confidence interval) dystrophin expression between DMD and non-dystrophic muscle (FIG. 3). With the average levels of dystrophin in patients with DMD measured as 5%, the present invention is consistent with previous reports that expression of endogenous dystrophin at levels >10% of those in healthy tissue, may be sufficient to protect from a more severe disease phenotype. Similarly, a mean of 32% dystrophin expression in patients with BMD was consistent with previous reports suggesting ˜30% of native dystrophin may be sufficient to reduce muscle weakness.

To evaluate the response to a given treatment, pre-treatment dystrophin levels need to be accurately quantified. As most patients with DMD produce trace amounts of dystrophin, or have revertant fibers, methods to quantify dystrophin must be sensitive enough to differentiate between low levels of expression. The methods of the present invention are able to detect very small differences in dystrophin expression and is sensitive enough to detect DMD revertant fibers. The methods of the present invention reflect the heterogeneity of dystrophin expression seen in patients with BMD or DMD and were relatively similar to those reported using a capillary Western immunoassay (BMD, 10-90%; DMD, 0.7-7%) (PLoS One 13(4):e0195850). In the same study, dystrophin expression in healthy human tissue ranged from 49-149% or 32-173%, depending on the antibody used, highlighting the importance of antibody selection in Western blot methods and consequently the potential for high variability.

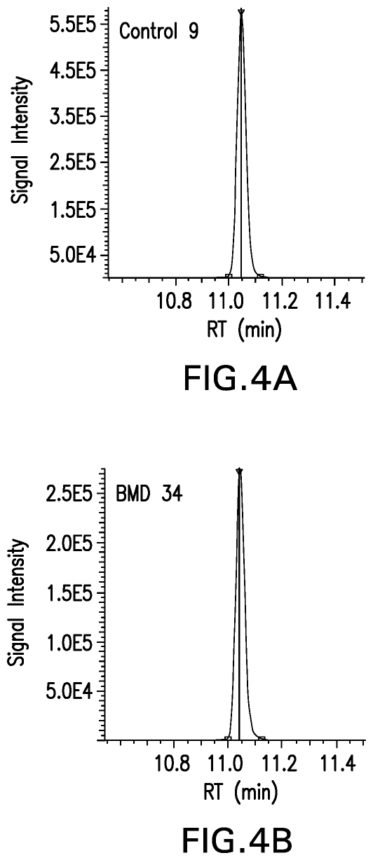

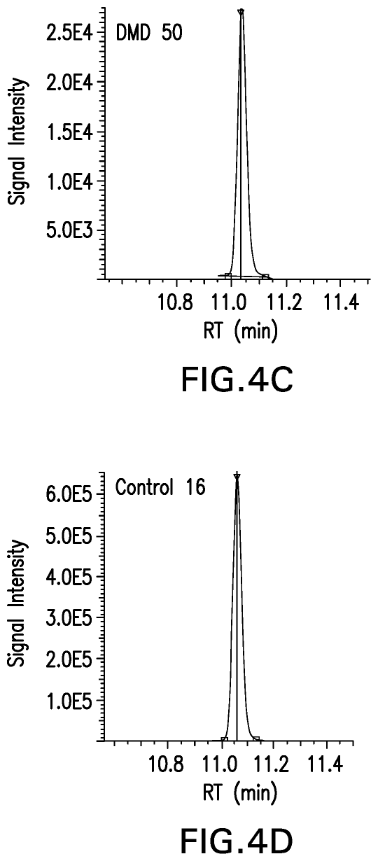

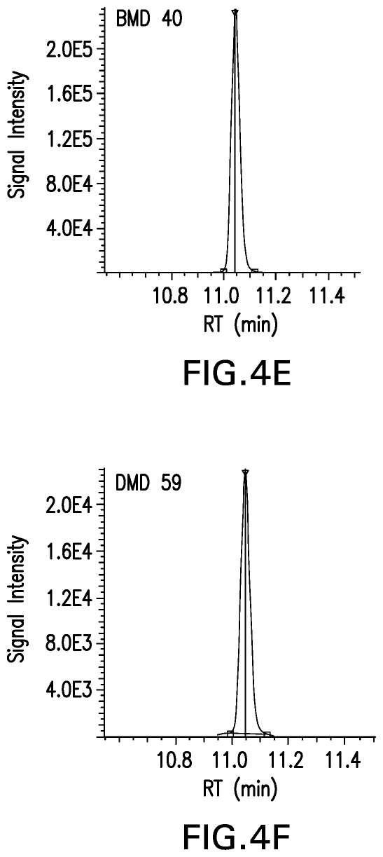

Representative LLQVAVEDR (SEQ ID NO:200) peptide extracted ion chromatograms from age-matched healthy, BMD, and DMD muscle samples are shown in FIGS. 4A-4F. As expected, peptide LEMPSSLMLEVPTHR (SEQ ID NO:236) was not detected in any of the samples.

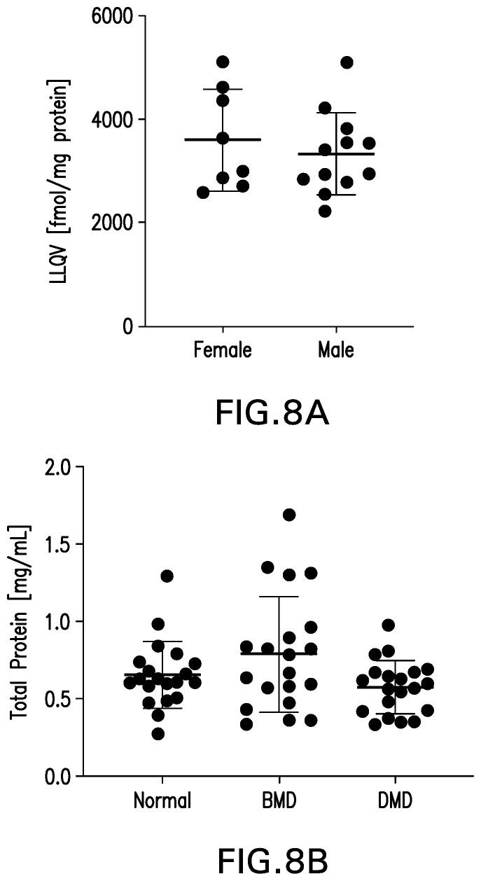

There was no apparent difference in dystrophin expression between healthy males and females (FIG. 8A). While mean total protein concentrations appeared to be consistent across healthy, BMD, and DMD muscle samples, the observed variability is a consequence of varying sizes of the tissue blocks from which the 10 um sections were cut (FIG. 8B).

### Example 9

### Method Validation

Validation samples for normal human tissue lysates were endogenous, 10× dilute endogenous and endogenous spiked with 833 fmol/mL engineered dystrophin protein. Human DMD validation samples were endogenous and endogenous spiked with 41.6 and 833 fmol/mL engineered dystrophin protein. Intra- and inter-assay precision were evaluated against acceptance criteria: overall precision (% coefficient of variation [CV]) and accuracy (% relative error) ≤25.0% (≤30% at LLOQ) for QC samples (low, medium, high). Stability studies assessed the lysate bench top stability, 72 h auto injector stability, 7-day processed sample stability, and 2 cycles of freeze-thaw at −70° C. The normal lysate pool, meanwhile, was assigned a concentration during the accuracy and precision portion of the validation.

### Example 10

### Quantification of Engineered Dystrophin Protein in Skeletal Muscle from DMD^{mdx} Rats Treated with an AAV9-Engineered-Dystrophin Candidate Gene Therapy

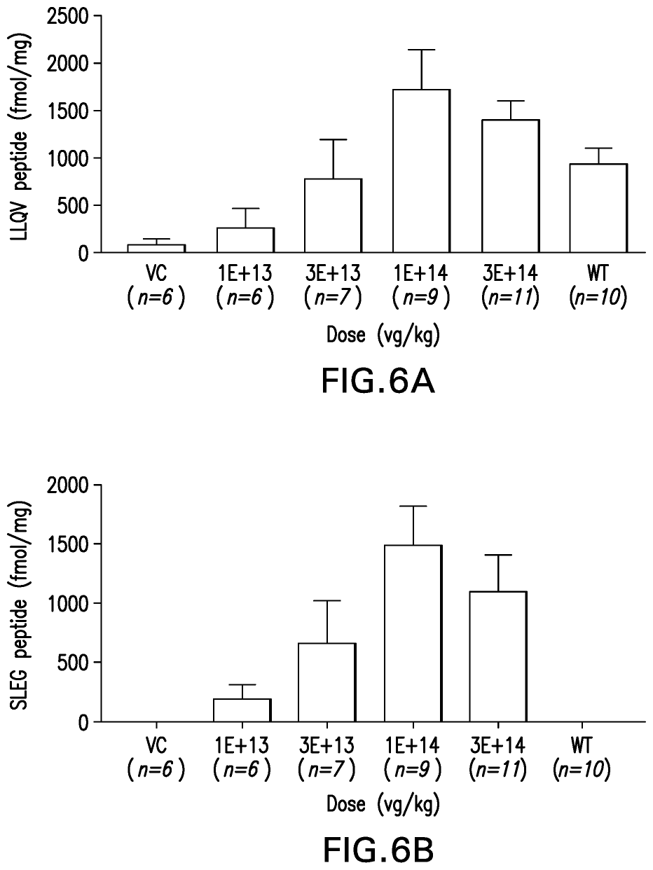

Endogenous full-length dystrophin and AAV-mediated expression of engineered dystrophin were quantified in bicep femoris muscle samples obtained from DMD^{mdx} rats (Larcher et al., PLoS One. 9, e110371 (2014)), after a single intravenous administration of AAV9.hCK.Hopti-Dys3978.spA in a dose-finding efficacy study. Results are presented for muscle samples collected at 6 mo post-administration for vehicle control rat groups and 4 DMD^{mdx} rat dose groups at 1×10¹³, 3×10¹³, 1×10¹⁴ and 3×10¹⁴ vector genome/kg of AAV9.hCK.Hopti-Dys3978.spA (FIG. 6A and FIG. 6B). Muscles samples were also harvested from untreated wild type (WT) rats. Common dystrophin peptide LLQVAVEDR (SEQ ID NO:200), which is conserved across human and rat, and detects total dystrophin (endogenous dystrophin (SEQ ID NO:242) and engineered dystrophin of SEQ ID NO:243); and SLEGSDDAVLLQR (SEQ ID NO:179), which is a human dystrophin-specific sequence that detects engineered dystrophin only, were quantified using the IA LC-MS/MS.

IA LC-MS/MS analysis of biceps femoris tissue samples from DMD^{mdx} rats treated with different doses (1×10¹³, 3×10¹³, 1×10¹⁴ and 3×10¹⁴ vg/kg) of a AAV9 vector encoding engineered dystrophin (AAV9.hCK.Hopti-Dys3978.spA) showed a dose-dependent increase in engineered dystrophin expression. At 6 months post-administration, the maximum engineered dystrophin expression was observed in the 1×10¹⁴ vector genome/kg (vg/kg) dose group (FIGS. 6A and 6B). Expression of SLEGSDDAVLLQR (SEQ ID NO:179) (engineered-dystrophin) and LLQVAVEDR (SEQ ID NO:200) (common dystrophin peptide) were comparable, with a maximum total dystrophin also observed in the 1×10¹⁴ vg/kg dose group (SEQ ID NO:179 can function as an engineered dystrophin peptide as it is not present in the endogenous dystrophin found in rat (see Table 11). Compared with WT rats, molar levels of engineered dystrophin in the 1×10¹⁴ vg/kg dose group surpassed normal levels of dystrophin by up to 150% normal. The concentration of revertant fiber dystrophin in DMD^{mdx} rats was 7.4%-10.4% of dystrophin expression in the WT rats. This allowed the quantification of dystrophin at the low femtomolar per milliliter level and was able to detect dystrophin in DMD muscle samples as low as approximately 1% relative to healthy human muscle. These results demonstrate the validity of the method across species and its potential use in preclinical and ultimately in clinical studies. This demonstrates the applicability of the methods of the invention in a preclinical DMD^{mdx} rat study, without the need for methodological changes, by showing a dose-dependent expression of mini-dystrophin protein 6 months post-administration of AAV9.hCK. Hopti-Dys3978.spA. The application of this assay in clinical trials can be invaluable in establishing the link between mini-dystrophin transgene expression and function, as well as providing guidance for dosing and method of administration.

### Example 11

### Peptide Selection and Tissue Processing

Targeted dystrophin and engineered dystrophin peptide sequences are summarized in FIG. 1B. Peptide LLQVAVEDR (SEQ ID NO:200), present in endogenous dystrophin and engineered dystrophin, is expressed in human, rattus, and canis species. Expression in a variety of species makes this target peptide and assay viable for use in clinical and preclinical investigations. Peptide SLEGSDDAVLLQR (SEQ ID NO:179), different in rattus and canis species but part of the human endogenous dystrophin and engineered dystrophin sequence, is a key peptide in preclinical studies reporting the transgene expressed engineered dystrophin. Peptide LEMPSSLMLEVPTHR (SEQ ID NO:236) is present in engineered dystrophin of SEQ ID NO:243 only and is used in clinical assessment of transgene protein expression.

Efficient protein extraction is essential for the large membrane-bound dystrophin protein. Tissue lysis with SDS was critical for dystrophin extraction, with optimal extraction achieved with 5% SDS in RIPA lysis buffer. SDS is commonly used in Western blot methods to enable efficient protein extraction and separation, but must be subsequently removed to prevent interference with protein-antibody binding or other downstream steps in an LC-MS assay. To this end, precipitation with an organic solvent removed not only SDS, but also OCT compound and potential contaminants. It also served to concentrate the sample. The use of OCT in muscle tissue samples had no effect on assay performance. Sections for LC-MS and tissue staining can therefore be taken from the same tissue block. Dystrophin expression is known to vary substantially between patients, with disease severity and type of mutation, as well as within different muscle type. The ability to analyze adjacent sections from the same tissue block may therefore improve the reliability of data interpretation. Although the presence of 5% SDS in the lysis buffer was used for the efficient extraction of dystrophin, any residual SDS should be removed prior to LC-MS analysis. Protein precipitation, washing the pellets with acetone and the anti-dystrophin peptide antibody column coupled online with LC-MS/MS allowed for effective removal of residual SDS and OCT, which can suppress the signal in LC-MS.

In contrast to Western blot methods, the use of a 96-well plate for lysate processing in this assay, with sufficient capacity to include calibrants and QC samples for reliable quantification, allowed for relatively large number of samples to be prepared simultaneously without the need to run multiple assays.

### Example 12

### IA LC-MS/MS Assay

SILAC engineered dystrophin (KempBio, Frederick, MD) was used as the internal standard, i.e., exogenous dystrophin reference protein. A high flow rate at the antibody column pump and the large binding capacity of the anti-dystrophin peptide antibody columns allowed a large volume of the processed sample to be loaded while taking advantage of the sensitivity gains provided by the analytical nanoflow chromatography and nanospray ionization on the mass spectrometer. This is important for the detection of low abundance proteins, such as dystrophin. A similar approach was successfully employed in the quantification of the human neonatal Fc receptor in transgenic mice and human tissues (Fan et al, Biomolecules. 2019; 9:373).

To further enhance mass spectrometric sensitivity and improve signal-to-noise, transition summing was used for all peptides. Transition summing can be easily applied to improve LLOQ by increasing peak area and averaging random noise.

### Example 13

### IA LC-MS/MS Assay Validation

Inter- and intra-assay accuracy and precision are summarized in Table 8. Peptides LEMPSSLMLEVPTHR (SEQ ID NO:236) and LLQVAVEDR (SEQ ID NO:200) met acceptance criteria and were successfully validated for the analysis of endogenous dystrophin and engineered dystrophin in human skeletal muscle sample lysates for the concentration ranges 6.67-3333 fmol/mL and 1.67-3333 fmol/mL, respectively. These results confirm high reproducibility of the method even for very low level detection of endogenous dystrophin and engineered dystrophin. The reproducibility of the method compares favorably with that of other techniques that are being developed for the quantification of dystrophin, including capillary Western immunoassays (Beekman et al., PLoS One. 13, e0195850 (2018)) and high-throughput immunofluorescence (Sardone et al., PLoS One. 13, e0194540 (2018)), and exceeds that reported for traditional Western blots (Schnell et al., US Neurol. 15, 40-46 (2019).).

Inter-assay accuracy and precision for LLQVAVEDR (SEQ ID NO:200) could only be assessed in spiked DMD samples where endogenous dystrophin was not detectable. Dystrophin concentration in each freshly prepared tissue sample was presumed to be different due to potential differences in tissue morphology.

Lysate samples were stable for 5 h at room temperature and after 4 weeks freeze-thaw at −70° C. Endogenous dystrophin was detected and levels were stable over a 5 month testing period following storage at −80° C. Assay sensitivity was maintained, confirming the stability of the calibrator.

TABLE 8
Inter- and intra-assay accuracy and precision
based on peptides LLQVAVEDR (SEQ ID NO:
200) and LEMPSSLMLEVPTHR (SEQ ID NO: 236)

| | Precision, (% CV) | | Accuracy (% RE) | |
|---|---|---|---|---|
| Peptide | Intra-assay | Inter-assay | Intra-assay | Inter-assay |
| SEQ ID NO: 200 | 1.6 to 12.6 | 5.1 to 8.8* | −12.1 to 9.9 | −5.7 to −2.4* |
| SEQ ID NO: 236 | 1.6 to 10.7 | 5.7 to 9.3 | −16.9 to 1.6 | −5.9 to −4.3 |

CV, coefficient of variation; DMD, Duchenne muscular dystrophy; RE, relative error.
*Assessment of inter-assay precision and accuracy for LLQVAVEDR (SEQ ID NO: 200) was only possible in spiked DMD validation samples where dystrophin was not detectable; endogenous dystrophin in normal tissue varied between runs as a fresh tissue section was used on each testing occasion.

### Example 14

### Dystrophin Expression in Normal, BMD, and DMD Muscles

Twenty skeletal muscle biopsy samples from healthy subjects and patients with BMD or DMD were obtained for dystrophin expression analysis. Approximately 90% of samples were biopsied from quadriceps muscle. One patient biopsy was collected from the gastrocnemius muscle and the muscle source of 4 samples was unknown. Healthy subject samples were collected from males (n=12) and females (n=8). Mean age (range) at the time of biopsy was 9.7 (4-16), 8.3 (3-19), and 6.0 (3-10) years for healthy subjects, patients with BMD, and those with DMD, respectively. Individual results from the dystrophin and total protein analysis in the lysate are available in Table 9 and the summary statistics are presented in Table 10.

Normal tissue dystrophin expression had a 23% CV with an expression range 61-148% of the normal mean expression (100%) based on the LLQVAVEDR (SEQ ID NO:200) peptide (FIG. 3). In BMD muscle, the expression range was 3-81% of normal mean (average 32%, n=20) and in DMD muscle, the expression range was 0.4-11.4% of normal mean (average 5%, n=16), with no dystrophin detected in 3 of 20 DMD samples. One patient with DMD had an in-frame mutation, generally characteristic of BMD, with a 28.1% expression level. Importantly, there was no overlap in dystrophin expression between DMD and healthy muscle (FIG. 3). The results are generally consistent with previous reports that expression of native dystrophin expression >10% of in healthy tissue, may be sufficient to protect from a more severe disease phenotype (van den Bergen et al., J. Neurol. Neurosurg. Psychiatry. 85, 747-753 (2014)), with the highest levels of dystrophin in patients with DMD measured as 11.4%. Similarly, a mean of 32% dystrophin expression in patients with BMD was consistent with previous reports suggesting that ˜30% of native of dystrophin may be sufficient to reduce muscle weakness (Neri et al., Neuromuscul. Disord. 17, 913-918 (2007)).

To evaluate the response to a given treatment, pre-treatment dystrophin levels need to be accurately quantified. As most patients with DMD produce trace amounts of dystrophin, or have revertant fibers, methods to quantify dystrophin must be sensitive enough to differentiate between low levels of expression. Previously reported LC-MS methods have been unable to resolve differences in dystrophin expression <5%. The current assay was able to detect very small differences in dystrophin expression and is sensitive enough to detect DMD revertant fibers. Our results reflect the heterogeneity of dystrophin expression seen in patients with BMD or DMD and were relatively similar to those reported using a capillary Western immunoassay (BMD, 10-90%; DMD, 0.7-7%) (Beekman et al., PLoS One. 13, e0195850 (2018)). In the same study, dystrophin expression in healthy human tissue ranged from 49-149% or 32-173%, depending on the antibody used, highlighting the importance of antibody selection in Western blot methods and consequently the potential for high variability.

There was a strong correlation between LLQVAVEDR (SEQ ID NO:200) and SLEGSDDAVLLQR (SEQ ID NO:179) peptides in healthy, BMD, and DMD muscle samples. As expected, peptide LEMPSSLMLEVPTHR (SEQ ID NO:236) was not detected in any of the samples.

There was no apparent difference in dystrophin expression between healthy males and females. Total protein expression appeared to be consistent across healthy, BMD, and DMD muscle samples.

The invention thus has been disclosed broadly and illustrated in reference to representative embodiments described above. Those skilled in the art will recognize that various modifications can be made to the present invention without departing from the spirit and scope thereof. All publications, patent applications, and issued patents, are herein incorporated by reference to the same extent as if each individual publication, patent application or issued patent were specifically and individually indicated to be incorporated by reference in its entirety. Definitions that are contained in text incorporated by reference are excluded to the extent that they contradict definitions in this disclosure.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. In particular, any aspect of the invention described in the claims, alone or in combination with one or more additional claims and/or aspects of the description, is to be understood as being combinable with other aspects of the invention set out elsewhere in the claims and/or description and/or sequence listings and/or drawings.

In so far as specific examples found herein do not fall within the scope of an invention, said specific example may be explicitly disclaimed.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value. For example, about can be ±10% of the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

The use of the term “or” in the claims is used to mean “and/or” unless explicitly indicated to refer to alternatives only or the alternatives are mutually exclusive, although the disclosure supports a definition that refers to only alternatives and “and/or.” As used herein the specification, “a” or “an” may mean one or more, unless clearly indicated otherwise. As used herein in the claim(s), when used in conjunction with the word “comprising”, the words “a” or “an” may mean one or more than one. As used herein “another” may mean at least a second or more. Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The words “comprises/comprising” and the words “having/including” when used herein with reference to the present invention are used to specify the presence of stated features, integers, steps or components but does not preclude the presence or addition of one or more other features, integers, steps, components or groups thereof.

Although the disclosed teachings have been described with reference to various applications, methods, and compositions, it will be appreciated that various changes and modifications can be made without departing from the teachings herein and the claimed invention below. The examples are provided to better illustrate the disclosed teachings and are not intended to limit the scope of the teachings presented herein. While the present teachings have been described in terms of these exemplary embodiments, numerous variations and modifications of these exemplary embodiments are possible without undue experimentation. All such variations and modifications are within the scope of the current teachings.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The description and examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

TABLE 9
Dystrophin and total protein analysis for A) control, non-
dystrophic subjects, B) BMD patients and C) DMD patients

| | | | | |
|---|---|---|---|---|
| | LCMS | BCA total | Dystrophin | Average |
| | dystrophin | protein | tissue | relative |
| | concen- | concen- | concen- | to |
| Anonymized | tration, | tration, | tration, | control |
| subject, # | fmol/mL | mg/mL | fmol/mg | group, % |

A)

| Control 1 | 1110 | 0.391 | 2838.9 | 82.5 |
|---|---|---|---|---|
| Control 2 | 2360 | 0.789 | 2991.1 | 86.9 |
| Control 3 | 1620 | 0.581 | 2788.3 | 81.1 |
| Control 4 | 1240 | 0.486 | 2551.4 | 74.2 |
| Control 5 | 2380 | 0.657 | 3622.5 | 105.3 |
| Control 6 | 3500 | 1.29 | 2713.2 | 78.9 |
| Control 7 | 3230 | 0.631 | 5118.9 | 148.8 |
| Control 8 | 2000 | 0.677 | 2954.2 | 85.9 |
| Control 9 | 2130 | 0.602 | 3538.2 | 102.9 |
| Control 10 | 2800 | 0.607 | 4612.9 | 134.1 |
| Control 11 | 2170 | 0.841 | 2580.3 | 75.0 |
| Control 12 | 1930 | 0.505 | 3821.8 | 111.1 |
| Control 13 | 2070 | 0.474 | 4367.1 | 126.9 |
| Control 14 | 772 | 0.27 | 2859.3 | 83.1 |
| Control 15 | 2880 | 0.983 | 2929.8 | 85.2 |
| Control 16 | 2620 | 0.737 | 3555.0 | 103.3 |
| Control 17 | 3210 | 0.629 | 5103.3 | 148.3 |
| Control 18 | 2470 | 0.724 | 3411.6 | 99.2 |
| Control 19 | 2520 | 0.598 | 4214.0 | 122.5 |
| Control 20 | 1350 | 0.605 | 2231.4 | 64.9 |
| | | Average, | | |
| | Average, % | fmol/ml | SD | CV, % |
| Overall | 100.0 | 3440.2 | 857.2 | 24.9 |

B)

| BMD 21 | 436 | 0.334 | 1305.4 | 37.9 |
|---|---|---|---|---|
| BMD 22 | 952 | 0.577 | 1649.9 | 48.0 |
| BMD 23 | 2430 | 0.836 | 2906.7 | 84.5 |
| BMD 24 | 1760 | 0.634 | 2776.0 | 80.7 |
| BMD 25 | 497 | 0.593 | 838.1 | 24.4 |
| BMD 26 | 72.1 | 0.473 | 152.4 | 4.4 |
| BMD 27 | 185 | 0.428 | 432.2 | 12.6 |
| BMD 28 | 115 | 0.36 | 319.4 | 9.3 |
| BMD 29 | 436 | 0.569 | 766.3 | 22.3 |
| BMD 30 | 55.5 | 0.36 | 154.2 | 4.5 |
| BMD 31 | 114 | 0.821 | 138.9 | 4.0 |
| BMD 32 | 788 | 1.3 | 606.2 | 17.6 |
| BMD 33 | 2130 | 0.78 | 2730.8 | 79.4 |
| BMD 34 | 1450 | 1.35 | 1074.1 | 31.2 |
| BMD 35 | 523 | 0.663 | 788.8 | 22.9 |
| BMD 36 | 1280 | 1.31 | 977.1 | 28.4 |
| BMD 37 | 2270 | 1.69 | 1343.2 | 39.0 |
| BMD 38 | 615 | 0.823 | 747.3 | 21.7 |
| BMD 39 | 918 | 0.96 | 956.3 | 27.8 |
| BMD 40 | 1000 | 0.891 | 1122.3 | 32.6 |
| | | Average, | | |
| | Average, % | fmol/ml | SD | CV, % |
| Overall | 31.7 | 1089.3 | 845.7 | 77.6 |

C)

| DMD 41 | 10* | 0.332 | 30.1 | 0.9 |
|---|---|---|---|---|
| DMD 42 | 44.7 | 0.617 | 72.4 | 2.1 |
| DMD 43 | 10* | 0.689 | 14.5 | 0.4 |
| DMD 44 | 70.9 | 0.477 | 148.6 | 4.3 |
| DMD 45 | 10* | 0.349 | 28.7 | 0.8 |
| DMD 46 | 268 | 0.973 | 275.4 | 8.0 |
| DMD 47 | 338 | 0.782 | 432.2 | 12.6 |
| DMD 48 | 49.6 | 0.643 | 77.1 | 2.2 |
| DMD 49 | 57.7 | 0.665 | 86.8 | 2.5 |
| DMD 50 | 105 | 0.598 | 175.6 | 5.1 |
| DMD 51 | 10* | 0.414 | 24.2 | 0.7 |
| DMD 52 | 10* | 0.544 | 18.4 | 0.5 |
| DMD 53 | 471 | 0.568 | 829.2 | 24.1 |
| DMD 54 | 10* | 0.374 | 26.7 | 0.8 |
| DMD 55 | 24.1 | 0.349 | 69.1 | 2.0 |
| DMD 56 | 24.1 | 0.56 | 43.0 | 1.3 |
| DMD 57 | 10* | 0.626 | 16.0 | 0.5 |
| DMD 58 | 221 | 0.808 | 273.5 | 8.0 |
| DMD 59 | 106 | 0.422 | 251.2 | 7.3 |
| DMD 60 | 555 | 0.673 | 824.7 | 24.0 |
| | | Average, | | |
| | Average, % | fmol/ml | SD | CV, % |
| Overall | 5.4 | 185.9 | 247.0 | 132.9 |

*Below limit of quantification (20.0 fmol/mL). Concentrations below the limit of quantification were imputed as 0.5*LLOQ for statistical analyses.
BCA, Bicinchoninic assay; BMD, Becker muscular dystrophy; CV, coefficient of variation; DMD, Duchenne muscular dystrophy; LCMS, liquid chromatography mass spectrometry; SD, standard deviation.

TABLE 10
Summary statistics of the tissue dystrophin concentration

| | | | | Tissue Dystrophin Concentration | | | | | |
|---|---|---|---|---|---|---|---|---|---|
| | | | | (fmol/mg) | | | | | |
| | | | | | | Bootstrap | Relative to Average of Control | | |
| | | | | | | 95% | Group (%) | | |
| | | | | | | Confidence | | | Bootstrap |
| | | Age (years) | | | | Interval | | | 95% |
| | | Median | Mean ± | Median | Mean ± | for the | Median | Mean ± | CI for the |
| Cohort | n | (Min, Max) | SD | (Min, Max) | SD | Mean* | (Min, Max) | SD | Mean* |
| DMD | 20 | 6 | 6 ± 1.8 | 74.8 | 185.9 ± 247.0 | (91, 299) | 2.2 | 5.4 ± 7.2 | (3, 9) |
| | | (3, 10) | | (14.5, 829.2) | | | (0.4, 24.1) | | |
| BMD | 20 | 8 | 8 ± 4.1 | 897.2 | 1089.3 ± 845.7 | (753, 1465) | 26.1 | 31.7 ± 24.6 | (22, 43) |
| | | (3, 19) | | (138.9, 2906.7) | | | (4.0, 84.5) | | |
| CTRL | 20 | 9 | 10 ± 4.3 | 3201.4 | 3440.2 ± 857.2 | (3091, 3824) | 93.1 | 100 ± 24.9 | (90, 111) |
| | | (4, 18) | | (2231.4, 5118.9) | | | (64.9, 148.8) | | |

*Random number generator state (random seed) was set at 2020 in R version 3.5.0 (43)
BMD, Becker muscular dystrophy; CI, confidence interval; CTRL, control; DMD, Duchenne muscular dystrophy; min, max, minimum, maximum; SD, standard deviation.

TABLE 11
Endogenous Dystrophin Peptides

| | | | | | | | | | | | Proteomics | | |
|---|---|---|---|---|---|---|---|---|---|---|---|---|---|
| | | | | | | | | | | | DB | | |
| | | | | | | In | | | | | Average | Predicted | |
| | Start- | | N | | C | Engineered | Conserved? | | | | PSM | Antigenicity | |
| No | End | Length | Term | Sequence | term | Dystrophin | Cyno | Mus | Rat | Dog | (>200 red) | Ranking | Comment |
| 1 | 1-13 | 13 | . | MLWWEEVEDCYER | E | Yes | X | X | X | X | NO | 4.7 | |
| 2 | 24-31 | 8 | K | WVNAQFSK | F | Yes | X | | | X | 22 | 1.8 | |
| 3 | 35-48 | 14 | K | QHIENLFSDL QDGR | R | Yes | X | | | X | 20 | 2.9 | 32-62 Missing in BMD |
| 4 | 50-61 | 12 | R | LLDLLEGLTG QK | L | Yes | X | X | X | X | 266 | 2.9 | 32-62 Missing in BMD |
| 5 | 71-79 | 9 | R | VHALNNVNK | A | Yes | X | X | X | X | 125 | 1.1 | |
| 6 | 83- | 23 | R | VLQNNNVDLVNIGSTDIV | L | Yes | X | | | | 110 | 2.7 | |
| | 105 | | | DG NHK | | | | | | | | | |
| 7 | 106- | 16 | K | LTLGLIWNII LHWQVK | N | Yes | X | X | X | X | N/O | 1.5 | |
| | 121 | | | | | | | | | | | | |
| 8 | 126- | 13 | K | NIMAGLQQTNSEK | I | Yes | X | | | X | 175 | 1.7 | |
| | 138 | | | | | | | | | | | | |
| 9 | 139- | 7 | K | ILLSWVR | Q | Yes | X | X | X | X | 549 | 1.7 | W not ideal, not |
| | 145 | | | | | | | | | | | | unique in human |
| | | | | | | | | | | | | | dystrophin |
| 10 | 178- | 18 | R | PDLFDWNSVVCQQSATQ | L | Yes | X | | | X | N/O | 3.1 | |
| | 195 | | | R | | | | | | | | | |
| 11 | 196- | 9 | R | LEHAFNIAR | Y | Yes | X | | | | 152 | 1.9 | |
| | 204 | | | | | | | | | | | | |
| 12 | 205- | 7 | R | YQLGIEK | L | Yes | X | | | X | 76 | 3.0 | |
| | 211 | | | | | | | | | | | | |
| 13 | 212- | 14 | K | LLDPEDVDTT YPDK | K | Yes | | | | | 27 | 4.4 | |
| | 225 | | | | | | | | | | | | |
| 14 | 263- | 26 | K | EEHFQLHHQM | T | Yes | X | | | X | N/O | 3.1 | |
| | 288 | | | HYSQQITVSL AQGYER | | | | | | | | | |
| 15 | 298- | 17 | K | SYAYTQAAYV TTSDPTR | S | Yes | X | | | X | 11 | 2.2 | |
| | 314 | | | | | | | | | | | | |
| 16 | 315- | 14 | R | SPFPSQHLEA PEDK | S | Yes | X | | | | 92 | 3.9 | |
| | 328 | | | | | | | | | | | | |
| 17 | 329- | 15 | K | SFGSSLMESE VNLDR | Y | Yes | X | | | | 76 | 2.6 | |
| | 343 | | | | | | | | | | | | |
| 18 | 344- | 33 | R | YQTALEEVLS | D | Yes | X | | | | N/O | 3.2 | Antigen.score: N- |
| | 376 | | | WLLSAEDTLQ | | | | | | | | | term: 3.2 C-term: 3.2 |
| | | | | AQGEISNDVE VVK | | | | | | | | | |
| 19 | 377- | 19 | K | DQFHTHEGYM | V | Yes | | | | | 9 | 3.3 | |
| | 395 | | | MDLTAHQGR | | | | | | | | | |
| 20 | 396- | 10 | R | VGNILQLGSK | L | Yes | | | | | 20 | 1.8 | |
| | 405 | | | | | | | | | | | | |
| 21 | 406- | 6 | K | LIGTGK | L | Yes | | | | | N/O | 2.3 | |
| | 411 | | | | | | | | | | | | |
| 22 | 412- | 19 | K | LSEDEETEVQ | W | Yes | X | | | X | 93 | 2.9 | |
| | 430 | | | EQMNLLNSR | | | | | | | | | |
| 23 | 436- | 6 | R | VASMEK | Q | Yes | X | X | X | X | N/O | 2.2 | |
| | 441 | | | | | | | | | | | | |
| 24 | 442- | 6 | K | QSNLHR | V | Yes | X | | | | N/O | 1.7 | |
| | 447 | | | | | | | | | | | | |
| 25 | 448- | 9 | R | VLMDLQNQK | L | Yes | X | X | X | | 520 | 1.9 | 3 x N demidation risk |
| | 456 | | | | | | | | | | | | |
| 26 | 459- | 8 | K | ELNDWLTK | T | Yes | X | | | X | 34 | 2.5 | |
| | 466 | | | | | | | | | | | | |
| 27 | 474- | 14 | K | MEEEPLGPDL EDLK | R | Yes | X | | X | | N/O | 4.5 | |
| | 487 | | | | | | | | | | | | |
| 28 | 489- | 6 | R | QVQQHK | V | Yes | X | | | X | N/O | 2.2 | |
| | 494 | | | | | | | | | | | | |
| 29 | 495 | 12 | K | VLQEDLEQEQVR | V | Yes | X | X | X | X | 1239 | 3.9 | 3 x Q demidation risk; |
| | 506 | | | | | | | | | | | | 495-534 Missing in |
| | | | | | | | | | | | | | BMD |
| 30 | 507- | 28 | R | VNSLTHMVW | V | Yes | X | X | X | X | 45 | 1.9 | 495-534 Missing in |
| | 534 | | | VDESSGDHAT AALEEQLK | | | | | | | | | BMD |
| 31 | 540- | 6 | R | WANICR | W | Yes | X | X | | X | 9 | 1.7 | |
| | 545 | | | | | | | | | | | | |
| 32 | 551- | 10 | R | WVLLQDILLK | W | No | X | | | X | 131 | 2.0 | |
| | 560 | | | | | | | | | | | | |
| 33 | 564- | 15 | R | LTEEQCLFSA WLSEK | E | No | X | | | | 8 | 2.3 | |
| | 578 | | | | | | | | | | | | |
| 34 | 579- | 6 | K | EDAVNK | I | No | | | | X | N/O | 3.8 | |
| | 584 | | | | | | | | | | | | |
| 35 | 585- | 7 | K | IHTTGFK | D | No | X | | | X | 69 | 2.0 | |
| | 591 | | | | | | | | | | | | |
| 36 | 592- | 11 | K | DQNEMLSSLQK | L | No | X | | | | 128 | 2.1 | |
| | 602 | | | | | | | | | | | | |
| 37 | 625- | 8 | K | QDLLSTLK | N | No | X | | | | 76 | 2.1 | |
| | 632 | | | | | | | | | | | | |
| 38 | 640- | 10 | K | TEAWLDNFAR | C | No | | | | | 121 | 2.3 | |
| | 649 | | | | | | | | | | | | |
| 39 | 650- | 8 | R | CWDNLVQK | L | No | X | | | | 36 | 2.1 | |
| | 657 | | | | | | | | | | | | |
| 40 | 661- | 31 | K | STAQISQAVT | E | No | | | | | N/O | 1.9 | Antigen score: N- |
| | 691 | | | TTQPSLTQTT | | | | | | | | | Term: 1.8 C-Term: 2.0 |
| | | | | VMETVTTVTT R | | | | | | | | | |
| 41 | 692- | 6 | R | EQILVK | H | No | X | | | | 12 | 2.5 | not unique in mouse |
| | 697 | | | | | | | | | | | | and rat dystrophin |
| 42 | 698- | 13 | K | HAQEELPPPP PQK | K | No | X | X | X | X | 50 | 2.2 | |
| | 710 | | | | | | | | | | | | |
| 43 | 713- | 9 | R | QITVDSEIR | K | No | X | | X | | 43 | 2.4 | |
| | 721 | | | | | | | | | | | | |
| 44 | 724- | 14 | R | LDVDITELHS WITR | S | No | X | X | X | X | 162 | 2.2 | |
| | 737 | | | | | | | | | | | | |
| 45 | 738- | 14 | R | SEAVLOSPEFA IFR | K | No | | | | | 1551 | 3.3 | not in MD |
| | 751 | | | | | | | | | | | | |
| 46 | 753- | 8 | K | EGNFSDLK | E | No | X | | | X | 14 | 3.3 | |
| | 760 | | | | | | | | | | | | |
| 47 | 763 | 6 | K | VNAIER | E | No | X | | | X | 21 | 2.2 | |
| | 768 | | | | | | | | | | | | |
| 48 | 777- | 6 | K | LQDASR | S | No | X | X | X | X | N/O | 2.5 | |
| | 782 | | | | | | | | | | | | |
| 49 | 783- | 20 | R | SAQALVEQMV | Q | No | | | | X | 111 | 2.9 | |
| | 802 | | | NEGVNADSIK | | | | | | | | | |
| 50 | 803- | 9 | K | QASEQLNSR | W | No | X | X | X | X | 11 | 2.0 | |
| | 811 | | | | | | | | | | | | |
| 51 | 812- | 11 | R | WIEFCOLLSER | L | No | X | | | X | 46 | 2.9 | |
| | 822 | | | | | | | | | | | | |
| 52 | 823- | 32 | R | LNWLEYQNNI | I | No | | | | | N/O | 2.7 | Antigen score: N- |
| | 854 | | | IAFYNQLQQL | | | | | | | | | term: 2.7 C-term: 2.8 |
| | | | | EQMTTTAENW LK | | | | | | | | | |
| 53 | 855- | 13 | K | IQPTTPSEPTAIK | S | No | X | | | | 2426 | 2.9 | not in MD |
| | 867 | | | | | | | | | | | | |
| 54 | 880- | 10 | R | LSDLQPQIER | L | No | | | | | 125 | \|2.7 | |
| | 889 | | | | | | | | | | | | |
| 55 | 892- | 7 | K | IQSIALK | E | No | | | | X | 198 | 1.7 | |
| | 898 | | | | | | | | | | | | |
| 56 | 901- | 19 | K | GQGPMFLDADFVAFTNH | Q | No | | | | | 66 | 3.2 | |
| | 919 | | | FK | | | | | | | | | |
| 57 | 920- | 9 | K | QVFSDVQAR | E | No | | | | | 121 | 2.2 | |
| | 928 | | | | | | | | | | | | |
| 58 | 931- | 13 | K | ELQTIFDTLPPMR | Y | No | X | X | X | | 18 | 3.0 | |
| | 943 | | | | | | | | | | | | |
| 59 | 944- | 9 | R | YQETMSAIR | T | No | X | | | | 249 | 2.2 | not in MD |
| | 952 | | | | | | | | | | | | |
| 50 | 953- | 9 | R | TWVQQSETK | L | No | X | | | | 14 | 2.2 | |
| | 961 | | | | | | | | | | | | |
| 61 | 962- | 17 | K | LSIPQLSVTDYEIMEQR | L | No | X | | | | 34 | 3.4 | |
| | 978 | | | | | | | | | | | | |
| 62 | 979- | 26 | R | LGELQALQSSLQEQQSGLY | E | No | | | | | 67 | 2.5 | |
| | 1004 | | | YLSTTVK | | | | | | | | | |
| 63 | 1010- | 7 | K | APSEISR | K | No | X | | | | 12 | 2.6 | |
| | 1016 | | | | | | | | | | | | |
| 64 | 1018- | 11 | K | YQSEFEEIEGR | W | No | | | | X | 25 | 4.5 | |
| | 1028 | | | | | | | | | | | | |
| 65 | 1032- | 11 | K | LSSQLVEHCQK | L | No | | | | X | 60 | 1.9 | |
| | 1042 | | | | | | | | | | | | |
| 66 | 1043- | 7 | K | LEEQMNK | L | No | X | | | | 48 | 3.6 | |
| | 1049 | | | | | | | | | | | | |
| 67 | 1053- | 9 | K | IQNHIQTLK | K | No | X | | | | 58 | 1.6 | |
| | 1061 | | | | | | | | | | | | |
| 68 | 1063- | 10 | K | WMAEVDVFLK | E | No | X | X | X | | N/O | 2.3 | |
| | 1072 | | | | | | | | | | | | |
| 59 | 1073- | 13 | K | EEWPALGDSEILK | K | No | X | | | X | 15 | 3.5 | |
| | 1085 | | | | | | | | | | | | |
| 70 | 1093- | 22 | R | LLVSDIQTIQPSLNSVNEG | I | No | | | | | 168 | 2.4 | |
| | 1114 | | | GQK | | | | | | | | | |
| 71 | 1117- | 10 | K | NEAEPEFASR | L | No | | | | | 21 | 3.9 | |
| | 1126 | | | | | | | | | | | | |
| 72 | 1127- | 6 | R | LETELK | E | No | | | | | N/O | 4.0 | not unique inmouse |
| | 1132 | | | | | | | | | | | | dystrophin |
| 73 | 1133- | 16 | K | ELNTQWDHMCQQVYAR | K | No | X | | | | 168 | 2.1 | |
| | 1148 | | | | | | | | | | | | |
| 74 | 1159- | 6 | K | TVSLQK | D | No | X | X | X | X | N/O | 1.8 | |
| | 1164 | | | | | | | | | | | | |
| 75 | 1165- | 19 | K | DLSEMHEWMTQAEEEYL | D | No | X | X | X | X | 18 | 4.3 | |
| | 1183 | | | ER | | | | | | | | | |
| 76 | 1189- | 7 | K | TPDELQK | A | No | X | | | | 77 | 3.8 | |
| | 119 | | | | | | | | | | | | |
| 77 | 1196- | 6 | K | AVEEMK | R | No | X | | | | N/O | 3.8 | |
| | 1201 | | | | | | | | | | | | |
| 78 | 1205- | 6 | K | EEAQQK | E | No | X | | | X | N/O | 4.3 | not unique in dog |
| | 1210 | | | | | | | | | | | | dystrophin |
| 79 | 1216- | 22 | K | LLTESVNSVIAQAPPVAQE | K | No | X | | | | 62 | 2.4 | |
| | 123 | | | ALK | | | | | | | | | |
| 80 | 1239- | 15 | K | ELETLTTNYQWLCTR | L | No | X | X | X | | 109 | 2.1 | |
| | 1253 | | | | | | | | | | | | |
| 81 | 1260- | 18 | K | TLEEVWACWHELLSYLEK | A | No | X | X | X | X | N/O | 2.4 | |
| | 1277 | | | | | | | | | | | | |
| 82 | 1281- | 8 | K | WLNEVEFK | L | No | X | | | | 64 | 3.1 | |
| | 1288 | | | | | | | | | | | | |
| 83 | 1291- | 24 | K | TTENIPGGAEEISEVLDSLE | H | No | | | | | 146 | 2.8 | |
| | 1314 | | | NLMR | | | | | | | | | |
| 84 | 1315- | 10 | R | HSEDNPNQIR | I | No | | | | | 170 | 2.4 | |
| | 1324 | | | | | | | | | | | | |
| 85 | 1325- | 26 | R | ILAQTLTDGGVMDELINEE | W | No | X | | | X | 30 | 3.2 | |
| | 1350 | | | LETFNSR | | | | | | | | | |
| 86 | 1353- | 8 | R | ELHEEAVR | R | No | X | X | X | X | 85 | 4.0 | |
| | 1360 | | | | | | | | | | | | |
| 87 | 1364- | 14 | K | LLEQSIQSAQETEK | S | No | | | | | 925 | 2.4 | |
| | 1377 | | | | | | | | | | | | |
| 88 | 1378- | 14 | K | SLHLIQESLTFIDK | Q | No | X | | | | 115 | 2.0 | |
| | 1391 | | | | | | | | | | | | |
| 89 | 1392- | 9 | K | QLAAYIADK | V | No | | | X | | 9 | 2.3 | |
| | 1400 | | | | | | | | | | | | |
| 90 | 1401- | 12 | K | VDAAQMPQEAQK | I | No | X | X | X | X | 92 | 2.4 | |
| | 1412 | | | | | | | | | | | | |
| 91 | 1413- | 16 | K | IQSDLTSHEISLEEMK | K | No | X | X | X | X | 25 | 2.3 | |
| | 1428 | | | | | | | | | | | | |
| 92 | 1440- | 10 | R | VLSQIDVAQK | K | No | X | X | X | X | 682 | 2.0 | not in MD |
| | 1449 | | | | | | | | | | | | |
| 93 | 1451- | 7 | K | LQDVSMK | F | No | X | X | | X | 33 | 2.1 | |
| | 1457 | | | | | | | | | | | | |
| 94 | 1464- | 7 | K | PANFEQR | L | No | X | X | X | X | 6 | 3.2 | not unique in human |
| | 1470 | | | | | | | | | | | | dystrophin |
| 95 | 1476- | 7 | K | MILDEVK | M | No | X | X | X | X | 94 | 3.6 | |
| | 1482 | | | | | | | | | | | | |
| 96 | 1483- | 9 | K | MHLPALETK | S | No | X | X | X | X | 58 | 2.1 | |
| | 1491 | | | | | | | | | | | | |
| 97 | 1492- | 19 | K | SVEQEVVQSQLNHCVNLY | S | No | X | | | X | 150 | 2.3 | |
| | 1510 | | | K | | | | | | | | | |
| 98 | 1511- | 6 | K | SLSEVK | S | No | X | X | X | X | N/O | 2.3 | |
| | 1516 | | | | | | | | | | | | |
| 99 | 1517- | 8 | K | SEVEMVIK | T | No | X | X | X | X | 82 | 2.4 | |
| | 1524 | | | | | | | | | | | | |
| 100 | 1534- | 6 | K | QTENPK | E | No | X | X | X | X | N/O | 2.8 | |
| | 1539 | | | | | | | | | | | | |
| 101 | 1550- | 9 | K | LHYNELGAK | V | No | X | X | X | X | 102 | 2.3 | |
| | 1558 | | | | | | | | | | | | |
| 102 | 1579- | 18 | K | EMNVLTEWLAATDMELT | R | No | X | | | | 162 | 2.0 | |
| | 1596 | | | K | | | | | | | | | |
| 103 | 1598- | 18 | R | SAVEGMPSNLDSEVAWG | A | No | X | X | X | X | 246 | 2.7 | not in MD |
| | 1615 | | | K | | | | | | | | | |
| 104 | 1630- | 10 | K | SITEVGEALK | T | No | X | | | | 59 | 2.9 | |
| | 1639 | | | | | | | | | | | | |
| 105 | 1646- | 7 | K | ETLVEDK | L | No | X | X | X | | 6 | 4.3 | |
| | 1652 | | | | | | | | | | | | |
| 106 | 1653- | 14 | K | LSLLNSNWIAVTSR | A | No | X | X | | X | 114 | 1.2 | |
| | 1666 | | | | | | | | | | | | |
| 107 | 1667- | 13 | R | AEEWLNLLLEYQK | H | No | X | | | X | 26 | 3.0 | |
| | 1679 | | | | | | | | | | | | |
| 108 | 1680- | 14 | K | HMETFDQNVDHITK | W | No | X | | | | 158 | 2.6 | |
| | 1693 | | | | | | | | | | | | |
| 109 | 1694- | 14 | K | WIIQADTLLDESEK | K | No | X | | | | 62 | 2.6 | |
| | 1707 | | | | | | | | | | | | |
| 110 | 1736- | 10 | R | DQAANLMANR | G | No | X | | | X | 133 | 1.6 | |
| | 1745 | | | | | | | | | | | | |
| 111 | 1752- | 12 | K | LVEPQISELNHR | F | No | X | | | | 6023 | 2.8 | not in MD |
| | 1763 | | | | | | | | | | | | |
| 112 | 1764- | 7 | R | FAAISHR | I | No | X | X | X | X | 40 | 1.7 | |
| | 1770 | | | | | | | | | | | | |
| 113 | 1776- | 6 | K | ASIPLK | E | No | X | X | X | X | N/O | 2.2 | |
| | 1781 | | | | | | | | | | | | |
| 114 | 1782- | 11 | K | ELEQFNSDIQK | L | No | X | X | X | X | 46 | 2.6 | |
| | 1792 | | | | | | | | | | | | |
| 115 | 1793- | 16 | K | LLEPLEAEIQQGVNLK | E | No | X | X | X | X | 75 | 2.9 | |
| | 1808 | | | | | | | | | | | | |
| 116 | 1809- | 6 | K | EEDFNK | D | No | X | X | X | X | N/O | 3.2 | |
| | 1814 | | | | | | | | | | | | |
| 117 | 1815- | 11 | K | DMNEDNEGTVK | E | No | X | | | | N/O | 4.2 | |
| | 1825 | | | | | | | | | | | | |
| 118 | 1831- | 7 | R | GDNLQQR | I | No | X | X | X | X | 24 | 3.1 | |
| | 1837 | | | | | | | | | | | | |
| 119 | 1851- | 7 | K | QQLLQTK | H | No | X | X | X | X | 29 | 1.5 | |
| | 1857 | | | | | | | | | | | | |
| 120 | 1872- | 12 | K | ALEISHQWYQYK | R | No | X | X | X | X | 46 | 2.4 | |
| | 1883 | | | | | | | | | | | | |
| 121 | 1885- | 7 | R | QADDLLK | C | No | X | X | | X | 12 | 3.9 | |
| | 1891 | | | | | | | | | | | | |
| 122 | 1892- | 7 | K | CLDDIEK | K | No | X | | | X | 17 | 4.3 | |
| | 1898 | | | | | | | | | | | | |
| 123 | 1900- | 8 | K | LASLPEPR | D | No | X | X | X | X | 194 | 3.8 | |
| | 1907 | | | | | | | | | | | | |
| 124 | 1924- | 7 | K | EELNAVR | R | No | X | X | X | X | 1174 | 3.6 | not in MD |
| | 1930 | | | | | | | | | | | | |
| 125 | 1932- | 23 | R | QAEGLSEDGAAMAVEPT | R | No | X | | | X | 156 | 3.3 | |
| | 1954 | | | QIQLSK | | | | | | | | | |
| 126 | 1969- | 10 | R | LNFAQIHTVR | E | No | X | | | | 141 | 1.5 | |
| | 1978 | | | | | | | | | | | | |
| 127 | 1979- | 48 | R | EETMMVMTEDMPLEISYV | D | No | | | | | N/O | 2.8 | Antigen score: N- |
| | 2026 | | | PSTYLTEITHVSQALLEVEQ | | | | | | | | | term: 2.6 C-term: 3.0 |
| | | | | LLNAPDLCAK | | | | | | | | | |
| 128 | 2027- | 7 | K | DFEDLFK | Q | No | X | X | X | | 72 | 3.6 | |
| | 2033 | | | | | | | | | | | | |
| 129 | 2034- | 6 | K | QEESLK | N | No | X | X | X | X | N/O | 4.2 | not unique in human |
| | 2039 | | | | | | | | | | | | dystrophin |
| 130 | 2043- | 9 | K | DSLQQSSGR | I | No | | | | | 13 | 2.3 | |
| | 2051 | | | | | | | | | | | | |
| 131 | 2052- | 7 | R | IDIIHSK | K | No | X | | | | 24 | 2.1 | |
| | 2058 | | | | | | | | | | | | |
| 132 | 2060- | 12 | K | TAALQSATPVER | V | No | X | | | | 175 | 2.0 | |
| | 2071 | | | | | | | | | | | | |
| 133 | 2074- | 14 | K | LQEALSQLDFQWEK | V | No | | | | | 101.4 | 3.0 | |
| | 2087 | | | | | | | | | | | | |
| 134 | 2109- | 6 | R | FHYDIK | I | No | X | | | | 15 | 2.5 | |
| | 2114 | | | | | | | | | | | | |
| 135 | 2115- | 14 | K | IFNQWLTEAEQFLR | K | No | X | | | | 156 | 2.1 | |
| | 2128 | | | | | | | | | | | | |
| 136 | 2130- | 11 | K | TQIPENWEHAK | Y | No | X | | | X | 5 | 3.0 | |
| | 2140 | | | | | | | | | | | | |
| 137 | 2147- | 9 | K | ELQDGIGQR | Q | No | X | X | X | X | 82 | 3.5 | |
| | 2155 | | | | | | | | | | | | |
| 138 | 2161- | 15 | R | TLNATGEEIIQQSSK | T | No | | X | X | | 2546.25 | 2.5 | not in MD |
| | 2175 | | | | | | | | | | | | |
| 139 | 2176- | 9 | K | TDASILQEK | L | No | X | | | X | 96 | 2.4 | |
| | 2184 | | | | | | | | | | | | |
| 140 | 2185- | 7 | K | LGSLNLR | W | No | X | | | X | 27 | 1.7 | |
| | 2191 | | | | | | | | | | | | |
| 141 | 2192- | 6 | R | WQEVCK | Q | No | X | | | X | 6 | 2.7 | |
| | 2197 | | | | | | | | | | | | |
| 142 | 2211- | 8 | K | NILSEFQR | D | No | X | | | X | 184.5 | 2.6 | |
| | 2218 | | | | | | | | | | | | |
| 143 | 2219- | 24 | R | DLNEFVLWLEEADNIASIPLE | E | No | | | | | N/O | 3.5 | |
| | 2242 | | | EPGK | | | | | | | | | |
| 144 | 2255- | 9 | K | LLVEELPLR | Q | No | X | | | | 2191.5 | 2.7 | not in MD |
| | 2263 | | | | | | | | | | | | |
| 145 | 2269- | 22 | K | QLNETGGPVLVSAPISPEE | L | No | X | | | | N/O | 3.0 | |
| | 2290 | | | QDK | | | | | | | | | |
| 146 | 2297- | 8 | K | QTNLQWIK | V | No | | X | X | X | 31.5 | 1.8 | |
| | 2304 | | | | | | | | | | | | |
| 147 | 2313- | 9 | K | QGEIEAQIK | D | No | X | | | | 50.25 | 3.3 | |
| | 2321 | | | | | | | | | | | | |
| 148 | 2322- | 7 | K | DLGQLEK | K | No | X | | | | 26 | 4.1 | |
| | 2328 | | | | | | | | | | | | |
| 149 | 2330- | 19 | K | LEDLEEQLNHLLLWLSPIR | N | No | X | | | | N/O | 2.8 | |
| | 2348 | | | | | | | | | | | | |
| 150 | 2349- | 18 | R | NQLEIYNQPNQEGPFDVK | E | No | | | | | 18 | 3.6 | |
| | 2366 | | | | | | | | | | | | |
| 151 | 2367- | 9 | K | ETEIAVQAK | Q | No | X | | | | 25 | 2.4 | |
| | 2375 | | | | | | | | | | | | |
| 152 | 2376- | 10 | K | QPDVEEILSK | G | No | | | | | 39.75 | 3.5 | |
| | 2385 | | | | | | | | | | | | |
| 153 | 2386- | 6 | K | GQHLYK | E | No | | X | X | X | N/O | 2.7 | |
| | 2391 | | | | | | | | | | | | |
| 154 | 2394- | 7 | K | PATQPVK | R | No | X | | | | N/O | 2.1 | |
| | 2400 | | | | | | | | | | | | |
| 155 | 2403- | 9 | K | LEDLSSEWK | A | No | X | | | | 6 | 3.9 | |
| | 2411 | | | | | | | | | | | | |
| 156 | 2416- | 6 | R | LLQELR | A | No | | | | | N/O | 2.5 | not unique in human |
| | 2421 | | | | | | | | | | | | dystrophin |
| 157 | 2424- | 29 | K | QPDLAPGLTTIGASPTQTV | E | No | | | | | N/O | 2.4 | |
| | 2452 | | | TLVTQPVVTK | | | | | | | | | |
| 158 | 2453- | 6 | K | ETAISK | L | Yes | X | | | X | N/O | 2.3 | |
| | 2458 | | | | | | | | | | | | |
| 159 | 2459- | 19 | K | LEMPSSLMLEVPALADEN | A | No | X | | | | 297 | 3.1 | not in MD |
| | 2477 | | | R | | | | | | | | | |
| 160 | 2478- | 17 | R | AWTELTDWLSLLDQVIK | S | No | | | | | 6 | 2.1 | |
| | 2494 | | | | | | | | | | | | |
| 161 | 2498- | 15 | R | VMVGDLEDINEMIIK | Q | No | X | X | X | X | 231 | 2.8 | not in MD |
| | 2512 | | | | | | | | | | | | |
| 162 | 2515- | 9 | K | ATMQDLEQR | R | No | | | | | 280 | 2.4 | not in MD |
| | 2523 | | | | | | | | | | | | |
| 163 | 2525- | 14 | R | PQLEELITAAQNLK | N | No | X | X | X | X | N/O | 2.1 | |
| | 2538 | | | | | | | | | | | | |
| 164 | 2541- | 7 | K | TSNQEAR | T | No | X | X | X | X | N/O | 2.1 | |
| | 2547 | | | | | | | | | | | | |
| 165 | 2548- | 6 | R | TIITDR | I | No | X | X | X | X | 12 | 2.3 | |
| | 2553 | | | | | | | | | | | | |
| 166 | 2557- | 15 | R | IQNOWDEVQEHLQNR | R | No | X | | | | 62 | 2.3 | |
| | 2571 | | | | | | | | | | | | |
| 167 | 2573- | 8 | R | QQLNEMLK | D | No | X | X | X | X | 1196 | 2.0 | not in MD |
| | 2580 | | | | | | | | | | | | |
| 168 | 2581- | 9 | K | DSTQWLEAK | E | No | X | X | X | X | 59 | 2.6 | |
| | 2589 | | | | | | | | | | | | |
| 169 | 2590- | 11 | K | EEAEQVLGQAR | A | No | X | | | X | 82 | 2.8 | |
| | 2600 | | | | | | | | | | | | |
| 170 | 2608- | 11 | K | EGPYTVDAIQK | K | No | | | | | 66 | 3.4 | |
| | 2618 | | | | | | | | | | | | |
| 171 | 2632- | 15 | R | QWQTNVDVANDLALK | L | No | | | | | 27 | 1.9 | |
| | 2646 | | | | | | | | | | | | |
| 172 | 2650- | 8 | R | DYSADDTR | K | No | X | X | X | X | 14 | 4.9 | |
| | 2657 | | | | | | | | | | | | |
| 173 | 2659- | 13 | K | VHMITENINASWR | S | No | | | | | 60 | 1.5 | |
| | 2671 | | | | | | | | | | | | |
| 174 | 2681- | 9 | R | EAALEETHR | L | No | | | | X | 33 | 3.8 | |
| | 2689 | | | | | | | | | | | | |
| 175 | 2690- | 11 | R | LLQQFPLDLEK | F | Yes | X | X | X | X | 194 | 3.1 | |
| | 2700 | | | | | | | | | | | | |
| 176 | 2701- | 20 | K | FLAWLTEAETTANVLQDA | K | Yes | X | | | | 121 | 2.4 | |
| | 2720 | | | TR | | | | | | | | | |
| 177 | 2724- | 6 | R | LLEDSK | G | Yes | X | | | X | 3 | 4.0 | |
| | 2729 | | | | | | | | | | | | |
| 178 | 2737- | 25 | K | QWQDLQGEIEAHTDVYH | I | Yes | X | | | | 28 | 3.4 | |
| | 2761 | | | NLDENSQK | | | | | | | | | |
| 179 | 2765- | 13 | R | SLEGSDDAVLLQR | R | Yes | X | | | | 1233 | 3.0 | |
| | 2777 | | | | | | | | | | | | |
| 180 | 2779- | 7 | R | LDNMNFK | W | Yes | X | X | X | X | 39 | 1.9 | |
| | 2785 | | | | | | | | | | | | |
| 181 | 2798- | 11 | R | SHLEASSDQWK | R | Yes | X | X | | X | 89 | 2.3 | |
| | 280 | | | | | | | | | | | | |
| 182 | 2810- | 15 | R | LHLSLQELLVWLQLK | D | Yes | X | X | X | X | N/O | 1.7 | |
| | 2824 | | | | | | | | | | | | |
| 183 | 2825- | 6 | K | DDELSR | Q | Yes | X | X | X | X | 3 | 3.7 | |
| | 2830 | | | | | | | | | | | | |
| 184 | 2831- | 13 | R | QAPIGGDFPAVQK | Q | Yes | | X | X | X | 107 | 3.6 | |
| | 2843 | | | | | | | | | | | | |
| 185 | 2844- | 6 | K | QNDVHR | A | Yes | X | | X | X | N/O | 2.3 | |
| | 2849 | | | | | | | | | | | | |
| 186 | 2859- | 12 | K | EPVIMSTLETVR | I | Yes | X | X | X | X | 33 | 2.7 | |
| | 2870 | | | | | | | | | | | | |
| 187 | 2871- | 13 | R | IFLTEQPLEGLEK | L | Yes | X | X | X | X | 168 | 3.3 | |
| | 2883 | | | | | | | | | | | | |
| 188 | 2884- | 6 | K | LYQEPR | E | Yes | X | X | X | X | 3 | 3.0 | |
| | 2889 | | | | | | | | | | | | |
| 189 | 2890- | 7 | R | ELPPEER | A | Yes | X | X | X | X | 7.5 | 3.6 | |
| | 2896 | | | | | | | | | | | | |
| 190 | 2897- | 6 | R | AQNVTR | L | Yes | X | X | X | X | N/O | 1.7 | |
| | 2902 | | | | | | | | | | | | |
| 191 | 2907- | 11 | K | QAEEVNTEWEK | L | Yes | X | | | | 6 | 3.9 | |
| | 2917 | | | | | | | | | | | | |
| 192 | 2918- | 10 | K | LNLHSADWQR | K | Yes | | | X | | 11 | 1.9 | in BMD 2921H→R |
| | 2927 | | | | | | | | | | | | |
| 193 | 2929- | 7 | K | IDETLER | L | Yes | X | | | | 8 | 4.3 | |
| | 2935 | | | | | | | | | | | | |
| 194 | 2936- | 14 | R | LQELQEATDELDLK | L | Yes | X | | | X | 149 | 3.9 | |
| | 2949 | | | | | | | | | | | | |
| 195 | 2952- | 6 | R | QAEVIK | G | Yes | X | X | X | X | 3 | 2.5 | |
| | 2957 | | | | | | | | | | | | |
| 196 | 2958- | 20 | K | GSWQPVGDLLIDSLQDHL | V | Yes | X | X | X | X | 64 | 3.5 | |
| | 2977 | | | EK | | | | | | | | | |
| 197 | 2983- | 7 | R | GEIAPLK | E | Yes | X | X | X | | 7 | 3.4 | |
| | 2989 | | | | | | | | | | | | |
| 198 | 2990- | 11 | K | ENVSHVNDLAR | Q | Yes | X | | | | 44 | 1.9 | |
| | 3000 | | | | | | | | | | | | |
| 199 | 3001- | 23 | R | QLTTLGIQLSPYNLSTLEDL | W | Yes | X | | | | 71 | 2.4 | |
| | 3023 | | | NTR | | | | | | | | | |
| 200 | 3026- | 9 | K | LLQVAVEDR | V | Yes | X | X | X | | 227 | 2.4 | |
| | 3034 | | | | | | | | | | | | |
| 201 | 3037- | 7 | R | QLHEAHR | D | Yes | X | X | X | X | N/O | 2.3 | |
| | 3043 | | | | | | | | | | | | |
| 202 | 3044- | 20 | R | DFGPASQHELSTSVQGPW | A | Yes | X | X | X | X | 54 | 3.4 | |
| | 3063 | | | ER | | | | | | | | | |
| 203 | 3064- | 6 | R | AISPNK | V | Yes | X | X | X | X | N/O | 2.0 | not unique in human |
| | 3069 | | | | | | | | | | | | dystrophin |
| 204 | 3070- | 18 | K | VPYYINHETQTTCWDHPK | M | Yes | X | X | X | X | 14 | 2.4 | |
| | 3087 | | | | | | | | | | | | |
| 205 | 3088- | 15 | K | MTELYQSLADLNNVR | F | Yes | X | X | X | X | 2850 | 1.9 | N-term N no ideal |
| | 3102 | | | | | | | | | | | | oxidation |
| 206 | 3118- | 22 | K | ALCLDLLSLSAACDALDQH | Q | Yes | X | X | X | X | 242 | 2.2 | long, multiple cys |
| | 3139 | | | NLK | | | | | | | | | |
| 207 | 3140- | 21 | K | QNDQPMDILQIINCLTTIY | L | Yes | X | X | X | | N/O | 3.0 | |
| | 3160 | | | DR | | | | | | | | | |
| 208 | 3161- | 30 | R | LEQEHNNLVNVPLCVDMC | T | Yes | X | X | X | X | N/O | 2.8 | |
| | 3190 | | | LNWLLNVYDTGR | | | | | | | | | |
| 209 | 3201- | 8 | K | TGIISLCK | A | Yes | X | X | X | X | 31 | 1.8 | |
| | 3208 | | | | | | | | | | | | |
| 210 | 3209- | 6 | K | AHLEDK | Y | Yes | X | X | X | X | N/O | 4.0 | |
| | 3214 | | | | | | | | | | | | |
| 211 | 3221- | 12 | K | QVASSTGFCDQR | R | Yes | | X | X | X | 409 | 2.6 | 2 x Q and 1 x C not |
| | 3232 | | | | | | | | | | | | ideal, but possible |
| 212 | 3234- | 13 | R | LGLLLHDSIQIPR | Q | Yes | X | X | X | X | 214 | 2.1 | |
| | 3246 | | | | | | | | | | | | |
| 213 | 3247- | 18 | R | QLGEVASFGGSNIEPSVR | S | Yes | X | X | X | X | 260 | 3.2 | too long |
| | 3264 | | | | | | | | | | | | |
| 214 | 3274- | 13 | K | PEIEAALFLDWMR | L | Yes | X | X | X | X | N/O | 3.0 | |
| | 3286 | | | | | | | | | | | | |
| 215 | 3287- | 14 | R | LEPQSMVWLPVLHR | V | Yes | X | X | X | X | 3993 | 2.1 | M and W oxidation, |
| | 3300 | | | | | | | | | | | | not unique in cyno, |
| | | | | | | | | | | | | | rat and dog |
| | | | | | | | | | | | | | dystrophin |
| 216 | 3301- | 8 | R | VAAAETAK | H | Yes | X | X | X | X | 123 | 2.0 | not unique in human |
| | 3308 | | | | | | | | | | | | dystrophin |
| 217 | 3318- | 8 | K | ECPIIGFR | Y | Yes | X | X | X | X | 78 | 3.3 | |
| | 3325 | | | | | | | | | | | | |
| 218 | 3331- | 15 | K | HFNYDICQSCFFSGR | V | Yes | X | X | X | X | 50 | 2.6 | |
| | 3345 | | | | | | | | | | | | |
| 219 | 3352- | 19 | K | MHYPMVEYCTPTTSGEDV | D | Yes | X | X | X | X | 63 | 3.5 | |
| | 3370 | | | R | | | | | | | | | |
| 220 | 3392- | 45 | R | MGYLPVQTVLEGDNMET | I | No | X | X | X | X | 5 | 2.5 | in BMD 3421A→V; |
| | 3436 | | | PVTLINFWPVDSAPASSPQ | | | | | | | | | antigen score: N- |
| | | | | LSHDDTHSR | | | | | | | | | term: 2.5 C-term: 2.6 |
| 221 | 3437- | 7 | R | IEHYASR | L | No | X | X | X | X | 29 | 2.6 | |
| | 3443 | | | | | | | | | | | | |
| 222 | 3444- | 46 | R | LAEMENSNGSYLNDSISPN | S | No | X | X | X | X | N/O | 2.7 | Antigen score: N- |
| | 3489 | | | ESIDDEHLLIQHYCQSLNQ | | | | | | | | | term: 2.6 C-term: 2.8 |
| | | | | DSPLSQPR | | | | | | | | | |
| 223 | 3490- | 14 | R | SPAQILISLESEER | G | No | X | X | X | X | 80 | 2.4 | |
| | 3503 | | | | | | | | | | | | |
| 224 | 3509- | 10 | R | ILADLEEENR | N | No | X | X | X | X | 965 | 3.9 | not in MD |
| | 3518 | | | | | | | | | | | | |
| 225 | 3519- | 8 | R | NLQAEYDR | L | No | X | X | X | X | 158 | 3.9 | |
| | 3526 | | | | | | | | | | | | |
| 226 | 3529- | 6 | K | QQHEHK | G | No | X | X | X | X | N/O | 2.7 | |
| | 3534 | | | | | | | | | | | | |
| 227 | 3535- | 20 | K | GLSPLPSPPEMMPTSPQS | D | No | X | X | X | X | 808 | 3.0 | double M prone to |
| | 3554 | | | PR | | | | | | | | | oxidation |
| 228 | 3555- | 9 | R | DAELIAEAK | L | No | X | X | X | X | 948 | 3.8 | not in MD |
| | 3563 | | | | | | | | | | | | |
| 229 | 3576- | 9 | R | MQILEDHNK | Q | No | X | X | X | X | 368 | 2.2 | not in MD, not unique |
| | 3584 | | | | | | | | | | | | in human dystrophin |
| 230 | 3585- | 8 | K | QLESQLHR | L | No | X | X | X | X | 234 | 2.3 | not in MD, not unique |
| | 3592 | | | | | | | | | | | | in human dystrophin |
| 231 | 3595- | 11 | R | QLLEQPQAEAK | V | No | X | X | X | X | 3366 | 2.6 | not in MD |
| | 3605 | | | | | | | | | | | | |
| 232 | 3606- | 15 | K | VNGTTVSSPSTSLQR | S | No | X | X | X | X | 194 | 1.6 | |
| | 3620 | | | | | | | | | | | | |
| 233 | 3621- | 10 | R | SDSSQPMLLR | V | No | X | X | IX | X | 1870 | 2.1 | not in MD |
| | 3630 | | | | | | | | | | | | |
| 234 | 3631- | 41 | R | VVGSQTSDSMGEEDLLSP | G | No | | | | | N/O | 3.3 | too long; Antigen |
| | 3671 | | | PQDTSTGLEEVMEQLNNS | | | | | | | | | score: N-term: 3.3 C- |
| | | | | FPSSR | | | | | | | | | term: 3.4 |

TABLE 12
Engineer Dystrophin Specific Peptides

| | | Predicted | |
|---|---|---|---|
| | Engineered | Antigenicity | Engineered Dystrophin |
| No | Dystrophin Peptde Sequence | Ranking | Protein |
| 235 | WVLLQDQPDLAPGLTTIGASPTQTVTLVTQPVV | (too large to | SEQ ID NO: 243 |
| | TK | predict, <31 aa) | |
| 236 | LEMPSSLMLEVPTHR | 2.1 | SEQ ID NO: 243 |
| 237 | MGYLPVQTVLEGDNMET | 3.2 | SEQ ID NO: 243 (new C-term) |
| 238 | QSNLHSYVPSTYLTEITHVSQALLEVEQLLNAPDL | (too large to | SEQ ID NO: 244 (Sp1-Sp16 |
| | CAK | predict, <31 aa) | junction) |
| 239 | LEEQSDQWK | 4.1 | SEQ ID NO: 244 (Sp17-Sp23 |
| | | | junction) |
| 240 | MGYLPVQTVLEGDNMETDTM | 3.2 | SEQ ID NO: 244 & SEQ ID |
| | | | NO: 245 (new C-term) |
| 241 | LQELTLER | 2.6 | SEQ ID NO :245 |

SEQUENCE OF FULL LENGTH HUMAN DYSTROPHIN (Uniprot ID: P11532)
(SEQ ID NO: 242)
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMKNI
MAGLQQTNSEKILLSWVRQSTRNYPQVNVINFTTSWSDGLALNALIHSHRPDLFDWNSVVCQQ
SATQRLEHAFNIARYQLGIEKLLDPEDVDTTYPDKKSILMYITSLFQVLPQQVSIEAIQEVEMLPR
PPKVTKEEHFQLHHQMHYSQQITVSLAQGYERTSSPKPRFKSYAYTQAAYVTTSDPTRSPFPS
QHLEAPEDKSFGSSLMESEVNLDRYQTALEEVLSWLLSAEDTLQAQGEISNDVEVVKDQFHTH
EGYMMDLTAHQGRVGNILQLGSKLIGTGKLSEDEETEVQEQMNLLNSRWECLRVASMEKQSN
LHRVLMDLQNQKLKELNDWLTKTEERTRKMEEEPLGPDLEDLKRQVQQHKVLQEDLEQEQVR
VNSLTHMVVVVDESSGDHATAALEEQLKVLGDRWANICRWTEDRWVLLQDILLKWQRLTEEQ
CLFSAWLSEKEDAVNKIHTTGFKDQNEMLSSLQKLAVLKADLEKKKQSMGKLYSLKQDLLSTLK
NKSVTQKTEAWLDNFARCWDNLVQKLEKSTAQISQAVTTTQPSLTQTTVMETVTTVTTREQILV
KHAQEELPPPPPQKKRQITVDSEIRKRLDVDITELHSWITRSEAVLQSPEFAIFRKEGNFSDLKE
KVNAIEREKAEKFRKLQDASRSAQALVEQMVNEGVNADSIKQASEQLNSRWIEFCQLLSERLN
WLEYQNNIIAFYNQLQQLEQMTTTAENWLKIQPTTPSEPTAIKSQLKICKDEVNRLSDLQPQIER
LKIQSIALKEKGQGPMFLDADFVAFTNHFKQVFSDVQAREKELQTIFDTLPPMRYQETMSAIRT
WVQQSETKLSIPQLSVTDYEIMEQRLGELQALQSSLQEQQSGLYYLSTTVKEMSKKAPSEISRK
YQSEFEEIEGRWKKLSSQLVEHCQKLEEQMNKLRKIQNHIQTLKKWMAEVDVFLKEEWPALGD
SEILKKQLKQCRLLVSDIQTIQPSLNSVNEGGQKIKNEAEPEFASRLETELKELNTQWDHMCQQ
VYARKEALKGGLEKTVSLQKDLSEMHEWMTQAEEEYLERDFEYKTPDELQKAVEEMKRAKEE
AQQKEAKVKLLTESVNSVIAQAPPVAQEALKKELETLTTNYQWLCTRLNGKCKTLEEVWACWH
ELLSYLEKANKWLNEVEFKLKTTENIPGGAEEISEVLDSLENLMRHSEDNPNQIRILAQTLTDGG
VMDELINEELETFNSRWRELHEEAVRRQKLLEQSIQSAQETEKSLHLIQESLTFIDKQLAAYIADK
VDAAQMPQEAQKIQSDLTSHEISLEEMKKHNQGKEAAQRVLSQIDVAQKKLQDVSMKFRLFQK
PANFEQRLQESKMILDEVKMHLPALETKSVEQEVVQSQLNHCVNLYKSLSEVKSEVEMVIKTG
RQIVQKKQTENPKELDERVTALKLHYNELGAKVTERKQQLEKCLKLSRKMRKEMNVLTEWLAA
TDMELTKRSAVEGMPSNLDSEVAWGKATQKEIEKQKVHLKSITEVGEALKTVLGKKETLVEDKL
SLLNSNWIAVTSRAEEWLNLLLEYQKHMETFDQNVDHITKWIIQADTLLDESEKKKPQQKEDVL
KRLKAELNDIRPKVDSTRDQAANLMANRGDHCRKLVEPQISELNHRFAAISHRIKTGKASIPLKE
LEQFNSDIQKLLEPLEAEIQQGVNLKEEDFNKDMNEDNEGTVKELLQRGDNLQQRITDERKRE
EIKIKQQLLQTKHNALKDLRSQRRKKALEISHQWYQYKRQADDLLKCLDDIEKKLASLPEPRDE
RKIKEIDRELQKKKEELNAVRRQAEGLSEDGAAMAVEPTQIQLSKRWREIESKFAQFRRLNFAQ
IHTVREETMMVMTEDMPLEISYVPSTYLTEITHVSQALLEVEQLLNAPDLCAKDFEDLFKQEESL
KNIKDSLQQSSGRIDIIHSKKTAALQSATPVERVKLQEALSQLDFQWEKVNKMYKDRQGRFDRS
VEKWRRFHYDIKIFNQWLTEAEQFLRKTQIPENWEHAKYKWYLKELQDGIGQRQTVVRTLNAT
GEEIIQQSSKTDASILQEKLGSLNLRWQEVCKQLSDRKKRLEEQKNILSEFQRDLNEFVLWLEE
ADNIASIPLEPGKEQQLKEKLEQVKLLVEELPLRQGILKQLNETGGPVLVSAPISPEEQDKLENKL
KQTNLQWIKVSRALPEKQGEIEAQIKDLGQLEKKLEDLEEQLNHLLLWLSPIRNQLEIYNQPNQE
GPFDVKETEIAVQAKQPDVEEILSKGQHLYKEKPATQPVKRKLEDLSSEWKAVNRLLQELRAK
QPDLAPGLTTIGASPTQTVTLVTQPVVTKETAISKLEMPSSLMLEVPALADFNRAWTELTDWLS
LLDQVIKSQRVMVGDLEDINEMIIKQKATMQDLEQRRPQLEELITAAQNLKNKTSNQEARTIITDR
IERIQNQWDEVQEHLQNRRQQLNEMLKDSTQWLEAKEEAEQVLGQARAKLESWKEGPYTVD
AIQKKITETKQLAKDLRQWQTNVDVANDLALKLLRDYSADDTRKVHMITENINASWRSIHKRVS
EREAALEETHRLLQQFPLDLEKFLAWLTEAETTANVLQDATRKERLLEDSKGVKELMKQWQDL
QGEIEAHTDVYHNLDENSQKILRSLEGSDDAVLLQRRLDNMNFKWSELRKKSLNIRSHLEASSD
QWKRLHLSLQELLVWLQLKDDELSRQAPIGGDFPAVQKQNDVHRAFKRELKTKEPVIMSTLET
VRIFLTEQPLEGLEKLYQEPRELPPEERAQNVTRLLRKQAEEVNTEWEKLNLHSADWQRKIDET
LERLQELQEATDELDLKLRQAEVIKGSWQPVGDLLIDSLQDHLEKVKALRGEIAPLKENVSHVN
DLARQLTTLGIQLSPYNLSTLEDLNTRWKLLQVAVEDRVRQLHEAHRDFGPASQHFLSTSVQG
PWERAISPNKVPYYINHETQTTCWDHPKMTELYQSLADLNNVRFSAYRTAMKLRRLQKALCLD
LLSLSAACDALDQHNLKQNDQPMDILQIINCLTTIYDRLEQEHNNLVNVPLCVDMCLNWLLNVY
DTGRTGRIRVLSFKTGIISLCKAHLEDKYRYLFKQVASSTGFCDQRRLGLLLHDSIQIPRQLGEV
ASFGGSNIEPSVRSCFQFANNKPEIEAALFLDWMRLEPQSMVWLPVLHRVAAAETAKHQAKCN
ICKECPIIGFRYRSLKHFNYDICQSCFFSGRVAKGHKMHYPMVEYCTPTTSGEDVRDFAKVLKN
KFRTKRYFAKHPRMGYLPVQTVLEGDNMETPVTLINFWPVDSAPASSPQLSHDDTHSRIEHYA
SRLAEMENSNGSYLNDSISPNESIDDEHLLIQHYCQSLNQDSPLSQPRSPAQILISLESEERGEL
ERILADLEEENRNLQAEYDRLKQQHEHKGLSPLPSPPEMMPTSPQSPRDAELIAEAKLLRQHK
GRLEARMQILEDHNKQLESQLHRLRQLLEQPQAEAKVNGTTVSSPSTSLQRSDSSQPMLLRVV
GSQTSDSMGEEDLLSPPQDTSTGLEEVMEQLNNSFPSSRGRNTPGKPMREDTM
SEQ ID NO: 243: SEQUENCE OF EXEMPLIFIED ENGINERED DYSTROPHIN
MLWWEEVEDCYEREDVQKKTFTKWVNAQFSKFGKQHIENLFSDLQDGRRLLDLLEGLTGQKL
PKEKGSTRVHALNNVNKALRVLQNNNVDLVNIGSTDIVDGNHKLTLGLIWNIILHWQVKNVMKNI
MAGLQQTNSEKILLSWVRQSTRNYPQVNVINFTTSWSDGLALNALIHSHRPDLFDWNSVVCQQ
SATQRLEHAFNIARYQLGIEKLLDPEDVDTTYPDKKSILMYITSLFQVLPQQVSIEAIQEVEMLPR
PPKVTKEEHFQLHHQMHYSQQITVSLAQGYERTSSPKPRFKSYAYTQAAYVTTSDPTRSPFPS
QHLEAPEDKSFGSSLMESEVNLDRYQTALEEVLSWLLSAEDTLQAQGEISNDVEVVKDQFHTH
EGYMMDLTAHQGRVGNILQLGSKLIGTGKLSEDEETEVQEQMNLLNSRWECLRVASMEKQSN
LHRVLMDLQNQKLKELNDWLTKTEERTRKMEEEPLGPDLEDLKRQVQQHKVLQEDLEQEQVR
VNSLTHMVVVVDESSGDHATAALEEQLKVLGDRWANICRWTEDRWVLLQDQPDLAPGLTTIG
ASPTQTVTLVTQPVVTKETAISKLEMPSSLMLEVPTHRLLQQFPLDLEKFLAWLTEAETTANVLQ
DATRKERLLEDSKGVKELMKQWQDLQGEIEAHTDVYHNLDENSQKILRSLEGSDDAVLLQRRL
DNMNFKWSELRKKSLNIRSHLEASSDQWKRLHLSLQELLVWLQLKDDELSRQAPIGGDFPAVQ
KQNDVHRAFKRELKTKEPVIMSTLETVRIFLTEQPLEGLEKLYQEPRELPPEERAQNVTRLLRK
QAEEVNTEWEKLNLHSADWQRKIDETLERLQELQEATDELDLKLRQAEVIKGSWQPVGDLLID
SLQDHLEKVKALRGEIAPLKENVSHVNDLARQLTTLGIQLSPYNLSTLEDLNTRWKLLQVAVED
RVRQLHEAHRDFGPASQHFLSTSVQGPWERAISPNKVPYYINHETQTTCWDHPKMTELYQSL
ADLNNVRFSAYRTAMKLRRLQKALCLDLLSLSAACDALDQHNLKQNDQPMDILQIINCLTTIYDR
LEQEHNNLVNVPLCVDMCLNWLLNVYDTGRTGRIRVLSFKTGIISLCKAHLEDKYRYLFKQVAS
STGFCDQRRLGLLLHDSIQIPRQLGEVASFGGSNIEPSVRSCFQFANNKPEIEAALFLDWMRLE
PQSMVWLPVLHRVAAAETAKHQAKCNICKECPIIGFRYRSLKHFNYDICQSCFFSGRVAKGHK
MHYPMVEYCTPTTSGEDVRDFAKVLKNKFRTKRYFAKHPRMGYLPVQTVLEGDNMET
SEQ ID NO: 244: SEQUENCE OF ENGINERED DYSTROPHIN (Translation of SEQ
ID NO: 1 from WO 2019/245973)
MLWWEEVEDCYEREDVQKKTFTKWVNAQFSKFGKQHIENLFSDLQDGRRLLDLLEGL
TGQKLPKEKGSTRVHALNNVNKALRVLQNNNVDLVNIGSTDIVDGNHKLTLGLIWNIIL
HWQVKNVMKNIMAGLQQTNSEKILLSWVRQSTRNYPQVNVINFTTSWSDGLALNALIH
SHRPDLFDWNSVVCQQSATQRLEHAFNIARYQLGIEKLLDPEDVDTTYPDKKSILMYITS
LFQVLPQQVSIEAIQEVEMLPRPPKVTKEEHFQLHHQMHYSQQITVSLAQGYERTSSPKP
RFKSYAYTQAAYVTTSDPTRSPFPSQHLEAPEDKSFGSSLMESEVNLDRYQTALEEVLS
WLLSAEDTLQAQGEISNDVEVVKDQFHTHEGYMMDLTAHQGRVGNILQLGSKLIGTGK
LSEDEETEVQEQMNLLNSRWECLRVASMEKQSNLHRVLMDLQNQKLKELNDWLTKTE
ERTRKMEEEPLGPDLEDLKRQVQQHKVLQEDLEQEQVRVNSLTHMVVVVDESSGDHA
TAALEEQLKVLGDRWANICRWTEDRWVLLQDILLKWQRLTEEQCLFSAWLSEKEDAV
NKIHTTGFKDQNEMLSSLQKLAVLKADLEKKKQSMGKLYSLKQDLLSTLKNKSVTQKT
EAWLDNFARCWDNLVQKLEKSTAQISQAVTTTQPSLTQTTVMETVTTVTTREQILVKH
AQEELPPPPPQKKRTLERLQELQEATDELDLKLRQAEVIKGSWQPVGDLLIDSLQDHLEK
VKALRGEIAPLKENVSHVNDLARQLTTLGIQLSPYNLSTLEDLNTRWKLLQVAVEDRVR
QLHEAHRDFGPASQHFLSTSVQGPWERAISPNKVPYYINHETQTTCWDHPKMTELYQSL
ADLNNVRFSAYRTAMKLRRLQKALCLDLLSLSAACDALDQHNLKQNDQPMDILQIINC
LTTIYDRLEQEHNNLVNVPLCVDMCLNWLLNVYDTGRTGRIRVLSFKTGIISLCKAHLE
DKYRYLFKQVASSTGFCDQRRLGLLLHDSIQIPRQLGEVASFGGSNIEPSVRSCFQFANN
KPEIEAALFLDWMRLEPQSMVWLPVLHRVAAAETAKHQAKCNICKECPIIGFRYRSLKH
FNYDICQSCFFSGRVAKGHKMHYPMVEYCTPTTSGEDVRDFAKVLKNKFRTKRYFAKH
PRMGYLPVQTVLEGDNMETDTM-
SEQ ID NO: 245: SEQUENCE OF ENGINERED DYSTROPHIN SFT-001 from
WO2016115543-BDC7126 (micro-dys5; Ck8e-uDys5, Seq ID 4)
MLWWEEVEDCYEREDVQKKTFTKWVNAQFSKFGKQHIENLFSDLQDGRRLLDLLEGL
TGQKLPKEKGSTRVHALNNVNKALRVLQNNNVDLVNIGSTDIVDGNHKLTLGLIWNIIL
HWQVKNVMKNIMAGLQQTNSEKILLSWVRQSTRNYPQVNVINFTTSWSDGLALNALIH
SHRPDLFDWNSVVCQQSATQRLEHAFNIARYQLGIEKLLDPEDVDTTYPDKKSILMYITS
LFQVLPQQVSIEAIQEVEMLPRPPKVTKEEHFQLHHQMHYSQQITVSLAQGYERTSSPKP
RFKSYAYTQAAYVTTSDPTRSPFPSQHLEAPEDKSFGSSLMESEVNLDRYQTALEEVLS
WLLSAEDTLQAQGEISNDVEVVKDQFHTHEGYMMDLTAHQGRVGNILQLGSKLIGTGK
LSEDEETEVQEQMNLLNSRWECLRVASMEKQSNLHSYVPSTYLTEITHVSQALLEVEQL
LNAPDLCAKDFEDLFKQEESLKNIKDSLQQSSGRIDIIHSKKTAALQSATPVERVKLQEAL
SQLDFQWEKVNKMYKDRQGRFDRSVEKWRRFHYDIKIFNQWLTEAEQFLRKTQIPENW
EHAKYKWYLKELQDGIGQRQTVVRTLNATGEEIIQQSSKTDASILQEKLGSLNLRWQEV
CKQLSDRKKRLEEQSDQWKRLHLSLQELLVWLQLKDDELSRQAPIGGDFPAVQKQNDV
HRAFKRELKTKEPVIMSTLETVRIFLTEQPLEGLEKLYQEPRELPPEERAQNVTRLLRKQ
AEEVNTEWEKLNLHSADWQRKIDETLERLQELQEATDELDLKLRQAEVIKGSWQPVGD
LLIDSLQDHLEKVKALRGEIAPLKENVSHVNDLARQLTTLGIQLSPYNLSTLEDLNTRWK
LLQVAVEDRVRQLHEAHRDFGPASQHELSTSVQGPWERAISPNKVPYYINHETQTTCW
DHPKMTELYQSLADLNNVRFSAYRTAMKLRRLQKALCLDLLSLSAACDALDQHNLKQ
NDQPMDILQIINCLTTIYDRLEQEHNNLVNVPLCVDMCLNWLLNVYDTGRTGRIRVLSF
KTGIISLCKAHLEDKYRYLFKQVASSTGFCDQRRLGLLLHDSIQIPRQLGEVASFGGSNIE
PSVRSCFQFANNKPEIEAALFLDWMRLEPQSMVWLPVLHRVAAAETAKHQAKCNICKE
CPIIGFRYRSLKHFNYDICQSCFFSGRVAKGHKMHYPMVEYCTPTTSGEDVRDFAKVLK
NKFRTKRYFAKHPRMGYLPVQTVLEGDNMETDTM

1. A method for measuring the amount of dystrophin protein in a protein sample isolated from a biological sample comprising:
(i) contacting the protein sample with a first protease forming a protein digest comprising dystrophin peptides;
(ii) applying the protein digest to a dystrophin peptide affinity reagent, wherein the dystrophin peptides in the protein digest are bound to the dystrophin peptide affinity reagent, and eluting the bound dystrophin peptides forming a dystrophin peptide enriched sample containing endogenous dystrophin peptides, engineered dystrophin peptides, or endogenous dystrophin peptides and engineered dystrophin peptides; and
(iii) performing liquid-chromatography-mass spectrometry (LC/MS) on the dystrophin peptide enriched sample.

2. The method as set forth in claim 1, wherein the biological sample is from a human patient having Duchenne's muscular dystrophy (DMD), and the human patient having DMD has been treated with a gene therapy encoding an engineered dystrophin protein.

3. The method as set forth in claim 1 or claim 2, wherein the endogenous dystrophin protein has an amino acid sequence that is at least 95% identical to the amino acid sequence of SEQ ID NO:242.

4. The method as set forth in any one of claims 1 to 3, wherein the dystrophin protein being measured is an engineered dystrophin protein.

5. The method as set forth in any one of claims 1 to 4, wherein the engineered dystrophin protein has an amino acid sequence that is at least 95% identical to an amino acid sequence selected from the group consisting of SEQ ID NO:243, SEQ ID NO:244, or SEQ ID NO:245.

6. The method as set forth in any one of claims 1 to 5, wherein the engineered dystrophin protein has an amino acid sequence that is at least 95% identical to the amino acid sequence of SEQ ID NO:243.

7. The method as set forth in any one of claims 1 to 6, wherein the engineered dystrophin protein has an amino acid sequence that is identical to the amino acid sequence of SEQ ID NO:243.

8. The method as set forth in any one of claims 1 to 7, wherein the protein sample further comprises a metabolically labeled exogenous dystrophin reference protein added to the protein sample.

9. The methods as set forth in claim 8, wherein the exogenous dystrophin reference protein has an amino acid sequence that shares at least 95% identity with the engineered dystrophin protein.

10. The method as set forth in any one of claim 1 to 9, wherein digestion of both the endogenous dystrophin protein and the engineered dystrophin protein by the first protease yields a common dystrophin peptide that is present in both an endogenous dystrophin protein and an engineered dystrophin protein.

11. The method as set forth in claim 10, wherein the common dystrophin peptide has the amino acid sequence of SLEGSDDAVLLQR (SEQ ID NO:179) or LLQVAVEDR (SEQ ID NO:200).

12. The method as set forth in any one of claim 1 to 11, wherein the engineered dystrophin peptide comprises a contiguous amino acid sequence that is present in the engineered dystrophin protein and not present in the endogenous dystrophin protein.

13. The method as set forth in any one of claim 1 to 12, wherein the engineered dystrophin-specific peptide has an amino acid sequence of SEQ ID NO:235, SEQ ID NO:236, SEQ ID NO:237, SEQ ID NO:238, SEQ ID NO:239, SEQ ID NO:240, or SEQ ID NO:241.

14. The method as set forth in any one of claim 1 to 13, wherein the engineered dystrophin-specific peptide has the amino acid sequence of LEMPSSLMLEVPTHR (SEQ ID NO:236).

15. The method as set forth in any one claim 1 to 14, wherein the biological sample is homogenized in a lysis buffer.

16. The method as set forth in any one of claim 15, wherein the lysis buffer includes a detergent.

17. The method as set forth in any one of claim 15 to 16, wherein the detergent is SDS.

18. The method as set forth in any one of claim 1 to 17, wherein the lysis buffer is a RIPA buffer, and RIPA buffer contains 10 to 50 mM Tris-HCl, 100 to 200 mM NaCl, 0.5 to 1.% NP40, 1 to 2% sodium deoxycholate and 0.1% SDS.

19. The method as set forth in any one of claim 1 to 18, wherein isolating the protein component of a biological sample includes contacting a homogenized biological sample with an organic solvent forming a protein precipitate.

20. The method as set forth in claim 19, wherein the organic solvent is selected from the group consisting of acetic acid, acetone, acetonitrile, dimethylformamide, dimethyl sulfoxide, dioxane, ethanol, isopropanol, methanol, 1-propanol, or tetrahydrofuran, or a combination thereof.

21. The method as set forth in any one of claims 1-20, wherein the first protease is provided in a protease buffer, and the protease buffer comprises at least a chaotropic agent, organic solvent and buffer salts, and preferably comprises about 0.8M urea and about 10% acetonitrile in phosphate buffered saline.

22. The method as set forth in any one of claim 1 to 21, wherein the dystrophin peptide affinity reagent binds at least one dystrophin peptide in the peptide sample.

23. The method as set forth in any one of claim 1 to 22, wherein the bound dystrophin peptides are from an endogenous dystrophin protein, an engineered dystrophin protein, and/or an exogenous dystrophin reference protein.

24. The method as set forth in any one of claim 1 to 23, wherein the dystrophin peptide affinity column binds a common dystrophin peptide that is present in both the endogenous dystrohin protein and the engineered dystrophin protein.

25. The method as set forth in any one of claim 1 to 24, wherein the dystrophin peptide affinity reagent comprises an anti-dystrophin peptide antibody.

26. The method as set forth in any one of claim 1 to 25, wherein the anti-dystrophin peptide antibody is raised against a peptide comprising an amino acid sequence selected from the group consisting of SEQ ID NO:235, SEQ ID NO:236, SEQ ID NO:237, SEQ ID NO:238, SEQ ID NO:239, SEQ ID NO:240, or SEQ ID NO:241.

27. The method as set forth in any one of claim 1 to 26, wherein the anti-dystrophin peptide antibody is raised against a peptide comprising the amino acid sequence of SEQ ID NO:236.

28. The method as set forth in any one of claim 1 to 27, wherein the anti-dystrophin peptide antibody is raised against a peptide comprising the amino acid sequence of LLQVAVEDR (SEQ ID NO:200).

29. The method as set forth in any one of claim 1 to 28, wherein the anti-dystrophin peptide antibody is a polyclonal antibody.

30. An anti-dystrophin peptide antibody raised against a peptide comprising an amino acid sequence selected from the group consisting of SEQ ID NO:235, SEQ ID NO:236, SEQ ID NO:237, SEQ ID NO:238, SEQ ID NO:239, SEQ ID NO:240, or SEQ ID NO:241.

31. A peptide comprising the amino acid sequence selected from the group consisting of SEQ ID NO:235, SEQ ID NO:236, SEQ ID NO:237, SEQ ID NO:238, SEQ ID NO:239, SEQ ID NO:240, or SEQ ID NO:241.


SEQUENCE LISTING (253 sequences)

SEQ ID NO: 1 - protein, 13 aa, Homo sapien
MLWWEEVEDCYER

SEQ ID NO: 2 - protein, 8 aa, Homo sapien
WVNAQFSK

SEQ ID NO: 3 - protein, 14 aa, Homo sapien
QHIENLFSDLQDGR

SEQ ID NO: 4 - protein, 12 aa, Homo sapien
LLDLLEGLTGQK

SEQ ID NO: 5 - protein, 9 aa, Homo sapien
VHALNNVNK

SEQ ID NO: 6 - protein, 23 aa, Homo sapien
VLQNNNVDLVNIGSTDIVDGNHK

SEQ ID NO: 7 - protein, 16 aa, Homo sapien
LTLGLIWNIILHWQVK

SEQ ID NO: 8 - protein, 13 aa, Homo sapien
NIMAGLQQTNSEK

SEQ ID NO: 9 - protein, 7 aa, Homo sapien
ILLSWVR

SEQ ID NO: 10 - protein, 18 aa, Homo sapien
PDLFDWNSVVCQQSATQR

SEQ ID NO: 11 - protein, 9 aa, Homo sapien
LEHAFNIAR

SEQ ID NO: 12 - protein, 7 aa, Homo sapien
YQLGIEK

SEQ ID NO: 13 - protein, 14 aa, Homo sapien
LLDPEDVDTTYPDK

SEQ ID NO: 14 - protein, 26 aa, Homo sapien
EEHFQLHHQMHYSQQITVSLAQGYER

SEQ ID NO: 15 - protein, 17 aa, Homo sapien
SYAYTQAAYVTTSDPTR

SEQ ID NO: 16 - protein, 14 aa, Homo sapien
SPFPSQHLEAPEDK

SEQ ID NO: 17 - protein, 15 aa, Homo sapien
SFGSSLMESEVNLDR

SEQ ID NO: 18 - protein, 33 aa, Homo sapien
YQTALEEVLSWLLSAEDTLQAQGEISNDVEVVK

SEQ ID NO: 19 - protein, 19 aa, Homo sapien
DQFHTHEGYMMDLTAHQGR

SEQ ID NO: 20 - protein, 10 aa, Homo sapien
VGNILQLGSK

SEQ ID NO: 21 - protein, 6 aa, Homo sapien
LIGTGK

SEQ ID NO: 22 - protein, 19 aa, Homo sapien
LSEDEETEVQEQMNLLNSR

SEQ ID NO: 23 - protein, 6 aa, Homo sapien
VASMEK

SEQ ID NO: 24 - protein, 6 aa, Homo sapien
QSNLHR

SEQ ID NO: 25 - protein, 9 aa, Homo sapien
VLMDLQNQK

SEQ ID NO: 26 - protein, 8 aa, Homo sapien
ELNDWLTK

SEQ ID NO: 27 - protein, 14 aa, Homo sapien
MEEEPLGPDLEDLK

SEQ ID NO: 28 - protein, 6 aa, Homo sapien
QVQQHK

SEQ ID NO: 29 - protein, 12 aa, Homo sapien
VLQEDLEQEQVR

SEQ ID NO: 30 - protein, 28 aa, Homo sapien
VNSLTHMVVVVDESSGDHATAALEEQLK

SEQ ID NO: 31 - protein, 6 aa, Homo sapien
WANICR

SEQ ID NO: 32 - protein, 10 aa, Homo sapien
WVLLQDILLK

SEQ ID NO: 33 - protein, 15 aa, Homo sapien
LTEEQCLFSAWLSEK

SEQ ID NO: 34 - protein, 6 aa, Homo sapien
EDAVNK

SEQ ID NO: 35 - protein, 7 aa, Homo sapien
IHTTGFK

SEQ ID NO: 36 - protein, 11 aa, Homo sapien
DQNEMLSSLQK

SEQ ID NO: 37 - protein, 8 aa, Homo sapien
QDLLSTLK

SEQ ID NO: 38 - protein, 10 aa, Homo sapien
TEAWLDNFAR

SEQ ID NO: 39 - protein, 8 aa, Homo sapien
CWDNLVQK

SEQ ID NO: 40 - protein, 31 aa, Homo sapien
STAQISQAVTTTQPSLTQTTVMETVTTVTTR

SEQ ID NO: 41 - protein, 6 aa, Homo sapien
EQILVK

SEQ ID NO: 42 - protein, 13 aa, Homo sapien
HAQEELPPPPPQK

SEQ ID NO: 43 - protein, 9 aa, Homo sapien
QITVDSEIR

SEQ ID NO: 44 - protein, 14 aa, Homo sapien
LDVDITELHSWITR

SEQ ID NO: 45 - protein, 14 aa, Homo sapien
SEAVLQSPEFAIFR

SEQ ID NO: 46 - protein, 8 aa, Homo sapien
EGNFSDLK

SEQ ID NO: 47 - protein, 6 aa, Homo sapien
VNAIER

SEQ ID NO: 48 - protein, 6 aa, Homo sapien
LQDASR

SEQ ID NO: 49 - protein, 20 aa, Homo sapien
SAQALVEQMVNEGVNADSIK

SEQ ID NO: 50 - protein, 9 aa, Homo sapien
QASEQLNSR

SEQ ID NO: 51 - protein, 11 aa, Homo sapien
WIEFCQLLSER

SEQ ID NO: 52 - protein, 32 aa, Homo sapien
LNWLEYQNNIIAFYNQLQQLEQMTTTAENWLK

SEQ ID NO: 53 - protein, 13 aa, Homo sapien
IQPTTPSEPTAIK

SEQ ID NO: 54 - protein, 10 aa, Homo sapien
LSDLQPQIER

SEQ ID NO: 55 - protein, 7 aa, Homo sapien
IQSIALK

SEQ ID NO: 56 - protein, 19 aa, Homo sapien
GQGPMFLDADFVAFTNHFK

SEQ ID NO: 57 - protein, 9 aa, Homo sapien
QVFSDVQAR

SEQ ID NO: 58 - protein, 13 aa, Homo sapien
ELQTIFDTLPPMR

SEQ ID NO: 59 - protein, 9 aa, Homo sapien
YQETMSAIR

SEQ ID NO: 60 - protein, 9 aa, Homo sapien
TWVQQSETK

SEQ ID NO: 61 - protein, 17 aa, Homo sapien
LSIPQLSVTDYEIMEQR

SEQ ID NO: 62 - protein, 26 aa, Homo sapien
LGELQALQSSLQEQQSGLYYLSTTVK

SEQ ID NO: 63 - protein, 7 aa, Homo sapien
APSEISR

SEQ ID NO: 64 - protein, 11 aa, Homo sapien
YQSEFEEIEGR

SEQ ID NO: 65 - protein, 11 aa, Homo sapien
LSSQLVEHCQK

SEQ ID NO: 66 - protein, 7 aa, Homo sapien
LEEQMNK

SEQ ID NO: 67 - protein, 9 aa, Homo sapien
IQNHIQTLK

SEQ ID NO: 68 - protein, 10 aa, Homo sapien
WMAEVDVFLK

SEQ ID NO: 69 - protein, 13 aa, Homo sapien
EEWPALGDSEILK

SEQ ID NO: 70 - protein, 22 aa, Homo sapien
LLVSDIQTIQPSLNSVNEGGQK

SEQ ID NO: 71 - protein, 10 aa, Homo sapien
NEAEPEFASR

SEQ ID NO: 72 - protein, 6 aa, Homo sapien
LETELK

SEQ ID NO: 73 - protein, 16 aa, Homo sapien
ELNTQWDHMCQQVYAR

SEQ ID NO: 74 - protein, 6 aa, Homo sapien
TVSLQK

SEQ ID NO: 75 - protein, 19 aa, Homo sapien
DLSEMHEWMTQAEEEYLER

SEQ ID NO: 76 - protein, 7 aa, Homo sapien
TPDELQK

SEQ ID NO: 77 - protein, 6 aa, Homo sapien
AVEEMK

SEQ ID NO: 78 - protein, 6 aa, Homo sapien
EEAQQK

SEQ ID NO: 79 - protein, 22 aa, Homo sapien
LLTESVNSVIAQAPPVAQEALK

SEQ ID NO: 80 - protein, 15 aa, Homo sapien
ELETLTTNYQWLCTR

SEQ ID NO: 81 - protein, 18 aa, Homo sapien
TLEEVWACWHELLSYLEK

SEQ ID NO: 82 - protein, 8 aa, Homo sapien
WLNEVEFK

SEQ ID NO: 83 - protein, 24 aa, Homo sapien
TTENIPGGAEEISEVLDSLENLMR

SEQ ID NO: 84 - protein, 10 aa, Homo sapien
HSEDNPNQIR

SEQ ID NO: 85 - protein, 26 aa, Homo sapien
ILAQTLTDGGVMDELINEELETFNSR

SEQ ID NO: 86 - protein, 8 aa, Homo sapien
ELHEEAVR

SEQ ID NO: 87 - protein, 14 aa, Homo sapien
LLEQSIQSAQETEK

SEQ ID NO: 88 - protein, 14 aa, Homo sapien
SLHLIQESLTFIDK

SEQ ID NO: 89 - protein, 9 aa, Homo sapien
QLAAYIADK

SEQ ID NO: 90 - protein, 12 aa, Homo sapien
VDAAQMPQEAQK

SEQ ID NO: 91 - protein, 16 aa, Homo sapien
IQSDLTSHEISLEEMK

SEQ ID NO: 92 - protein, 10 aa, Homo sapien
VLSQIDVAQK

SEQ ID NO: 93 - protein, 7 aa, Homo sapien
LQDVSMK

SEQ ID NO: 94 - protein, 7 aa, Homo sapien
PANFEQR

SEQ ID NO: 95 - protein, 7 aa, Homo sapien
MILDEVK

SEQ ID NO: 96 - protein, 9 aa, Homo sapien
MHLPALETK

SEQ ID NO: 97 - protein, 19 aa, Homo sapien
SVEQEVVQSQLNHCVNLYK

SEQ ID NO: 98 - protein, 6 aa, Homo sapien
SLSEVK

SEQ ID NO: 99 - protein, 8 aa, Homo sapien
SEVEMVIK

SEQ ID NO: 100 - protein, 6 aa, Homo sapien
QTENPK

SEQ ID NO: 101 - protein, 9 aa, Homo sapien
LHYNELGAK

SEQ ID NO: 102 - protein, 18 aa, Homo sapien
EMNVLTEWLAATDMELTK

SEQ ID NO: 103 - protein, 18 aa, Homo sapien
SAVEGMPSNLDSEVAWGK

SEQ ID NO: 104 - protein, 10 aa, Homo sapien
SITEVGEALK

SEQ ID NO: 105 - protein, 7 aa, Homo sapien
ETLVEDK

SEQ ID NO: 106 - protein, 14 aa, Homo sapien
LSLLNSNWIAVTSR

SEQ ID NO: 107 - protein, 13 aa, Homo sapien
AEEWLNLLLEYQK

SEQ ID NO: 108 - protein, 14 aa, Homo sapien
HMETFDQNVDHITK

SEQ ID NO: 109 - protein, 14 aa, Homo sapien
WIIQADTLLDESEK

SEQ ID NO: 110 - protein, 10 aa, Homo sapien
DQAANLMANR

SEQ ID NO: 111 - protein, 12 aa, Homo sapien
LVEPQISELNHR

SEQ ID NO: 112 - protein, 7 aa, Homo sapien
FAAISHR

SEQ ID NO: 113 - protein, 6 aa, Homo sapien
ASIPLK

SEQ ID NO: 114 - protein, 11 aa, Homo sapien
ELEQFNSDIQK

SEQ ID NO: 115 - protein, 16 aa, Homo sapien
LLEPLEAEIQQGVNLK

SEQ ID NO: 116 - protein, 6 aa, Homo sapien
EEDFNK

SEQ ID NO: 117 - protein, 11 aa, Homo sapien
DMNEDNEGTVK

SEQ ID NO: 118 - protein, 7 aa, Homo sapien
GDNLQQR

SEQ ID NO: 119 - protein, 7 aa, Homo sapien
QQLLQTK

SEQ ID NO: 120 - protein, 12 aa, Homo sapien
ALEISHQWYQYK

SEQ ID NO: 121 - protein, 7 aa, Homo sapien
QADDLLK

SEQ ID NO: 122 - protein, 7 aa, Homo sapien
CLDDIEK

SEQ ID NO: 123 - protein, 8 aa, Homo sapien
LASLPEPR

SEQ ID NO: 124 - protein, 7 aa, Homo sapien
EELNAVR

SEQ ID NO: 125 - protein, 23 aa, Homo sapien
QAEGLSEDGAAMAVEPTQIQLSK

SEQ ID NO: 126 - protein, 10 aa, Homo sapien
LNFAQIHTVR

SEQ ID NO: 127 - protein, 48 aa, Homo sapien
EETMMVMTEDMPLEISYVPSTYLTEITHVSQALLEVEQLLNAPDLCAK

SEQ ID NO: 128 - protein, 7 aa, Homo sapien
DFEDLFK

SEQ ID NO: 129 - protein, 6 aa, Homo sapien
QEESLK

SEQ ID NO: 130 - protein, 9 aa, Homo sapien
DSLQQSSGR

SEQ ID NO: 131 - protein, 7 aa, Homo sapien
IDIIHSK

SEQ ID NO: 132 - protein, 12 aa, Homo sapien
TAALQSATPVER

SEQ ID NO: 133 - protein, 14 aa, Homo sapien
LQEALSQLDFQWEK

SEQ ID NO: 134 - protein, 6 aa, Homo sapien
FHYDIK

SEQ ID NO: 135 - protein, 14 aa, Homo sapien
IFNQWLTEAEQFLR

SEQ ID NO: 136 - protein, 11 aa, Homo sapien
TQIPENWEHAK

SEQ ID NO: 137 - protein, 9 aa, Homo sapien
ELQDGIGQR

SEQ ID NO: 138 - protein, 15 aa, Homo sapien
TLNATGEEIIQQSSK

SEQ ID NO: 139 - protein, 9 aa, Homo sapien
TDASILQEK

SEQ ID NO: 140 - protein, 7 aa, Homo sapien
LGSLNLR

SEQ ID NO: 141 - protein, 6 aa, Homo sapien
WQEVCK

SEQ ID NO: 142 - protein, 8 aa, Homo sapien
NILSEFQR

SEQ ID NO: 143 - protein, 24 aa, Homo sapien
DLNEFVLWLEEADNIASIPLEPGK

SEQ ID NO: 144 - protein, 9 aa, Homo sapien
LLVEELPLR

SEQ ID NO: 145 - protein, 22 aa, Homo sapien
QLNETGGPVLVSAPISPEEQDK

SEQ ID NO: 146 - protein, 8 aa, Homo sapien
QTNLQWIK

SEQ ID NO: 147 - protein, 9 aa, Homo sapien
QGEIEAQIK

SEQ ID NO: 148 - protein, 7 aa, Homo sapien
DLGQLEK

SEQ ID NO: 149 - protein, 19 aa, Homo sapien
LEDLEEQLNHLLLWLSPIR

SEQ ID NO: 150 - protein, 18 aa, Homo sapien
NQLEIYNQPNQEGPFDVK

SEQ ID NO: 151 - protein, 9 aa, Homo sapien
ETEIAVQAK

SEQ ID NO: 152 - protein, 10 aa, Homo sapien
QPDVEEILSK

SEQ ID NO: 153 - protein, 6 aa, Homo sapien
GQHLYK

SEQ ID NO: 154 - protein, 7 aa, Homo sapien
PATQPVK

SEQ ID NO: 155 - protein, 9 aa, Homo sapien
LEDLSSEWK

SEQ ID NO: 156 - protein, 6 aa, Homo sapien
LLQELR

SEQ ID NO: 157 - protein, 29 aa, Homo sapien
QPDLAPGLTTIGASPTQTVTLVTQPVVTK

SEQ ID NO: 158 - protein, 6 aa, Homo sapien
ETAISK

SEQ ID NO: 159 - protein, 19 aa, Homo sapien
LEMPSSLMLEVPALADFNR

SEQ ID NO: 160 - protein, 17 aa, Homo sapien
AWTELTDWLSLLDQVIK

SEQ ID NO: 161 - protein, 15 aa, Homo sapien
VMVGDLEDINEMIIK

SEQ ID NO: 162 - protein, 9 aa, Homo sapien
ATMQDLEQR

SEQ ID NO: 163 - protein, 14 aa, Homo sapien
PQLEELITAAQNLK

SEQ ID NO: 164 - protein, 7 aa, Homo sapien
TSNQEAR

SEQ ID NO: 165 - protein, 6 aa, Homo sapien
TIITDR

SEQ ID NO: 166 - protein, 15 aa, Homo sapien
IQNQWDEVQEHLQNR

SEQ ID NO: 167 - protein, 8 aa, Homo sapien
QQLNEMLK

SEQ ID NO: 168 - protein, 9 aa, Homo sapien
DSTQWLEAK

SEQ ID NO: 169 - protein, 11 aa, Homo sapien
EEAEQVLGQAR

SEQ ID NO: 170 - protein, 11 aa, Homo sapien
EGPYTVDAIQK

SEQ ID NO: 171 - protein, 15 aa, Homo sapien
QWQTNVDVANDLALK

SEQ ID NO: 172 - protein, 8 aa, Homo sapien
DYSADDTR

SEQ ID NO: 173 - protein, 13 aa, Homo sapien
VHMITENINASWR

SEQ ID NO: 174 - protein, 9 aa, Homo sapien
EAALEETHR

SEQ ID NO: 175 - protein, 11 aa, Homo sapien
LLQQFPLDLEK

SEQ ID NO: 176 - protein, 20 aa, Homo sapien
FLAWLTEAETTANVLQDATR

SEQ ID NO: 177 - protein, 6 aa, Homo sapien
LLEDSK

SEQ ID NO: 178 - protein, 25 aa, Homo sapien
QWQDLQGEIEAHTDVYHNLDENSQK

SEQ ID NO: 179 - protein, 13 aa, Homo sapien
SLEGSDDAVLLQR

SEQ ID NO: 180 - protein, 7 aa, Homo sapien
LDNMNFK

SEQ ID NO: 181 - protein, 11 aa, Homo sapien
SHLEASSDQWK

SEQ ID NO: 182 - protein, 15 aa, Homo sapien
LHLSLQELLVWLQLK

SEQ ID NO: 183 - protein, 6 aa, Homo sapien
DDELSR

SEQ ID NO: 184 - protein, 13 aa, Homo sapien
QAPIGGDFPAVQK

SEQ ID NO: 185 - protein, 6 aa, Homo sapien
QNDVHR

SEQ ID NO: 186 - protein, 12 aa, Homo sapien
EPVIMSTLETVR

SEQ ID NO: 187 - protein, 13 aa, Homo sapien
IFLTEQPLEGLEK

SEQ ID NO: 188 - protein, 6 aa, Homo sapien
LYQEPR

SEQ ID NO: 189 - protein, 7 aa, Homo sapien
ELPPEER

SEQ ID NO: 190 - protein, 6 aa, Homo sapien
AQNVTR

SEQ ID NO: 191 - protein, 11 aa, Homo sapien
QAEEVNTEWEK

SEQ ID NO: 192 - protein, 10 aa, Homo sapien
LNLHSADWQR

SEQ ID NO: 193 - protein, 7 aa, Homo sapien
IDETLER

SEQ ID NO: 194 - protein, 14 aa, Homo sapien
LQELQEATDELDLK

SEQ ID NO: 195 - protein, 6 aa, Homo sapien
QAEVIK

SEQ ID NO: 196 - protein, 20 aa, Homo sapien
GSWQPVGDLLIDSLQDHLEK

SEQ ID NO: 197 - protein, 7 aa, Homo sapien
GEIAPLK

SEQ ID NO: 198 - protein, 11 aa, Homo sapien
ENVSHVNDLAR

SEQ ID NO: 199 - protein, 23 aa, Homo sapien
QLTTLGIQLSPYNLSTLEDLNTR

SEQ ID NO: 200 - protein, 9 aa, Homo sapien
LLQVAVEDR

SEQ ID NO: 201 - protein, 7 aa, Homo sapien
QLHEAHR

SEQ ID NO: 202 - protein, 20 aa, Homo sapien
DFGPASQHFLSTSVQGPWER

SEQ ID NO: 203 - protein, 6 aa, Homo sapien
AISPNK

SEQ ID NO: 204 - protein, 18 aa, Homo sapien
VPYYINHETQTTCWDHPK

SEQ ID NO: 205 - protein, 15 aa, Homo sapien
MTELYQSLADLNNVR

SEQ ID NO: 206 - protein, 22 aa, Homo sapien
ALCLDLLSLSAACDALDQHNLK

SEQ ID NO: 207 - protein, 21 aa, Homo sapien
QNDQPMDILQIINCLTTIYDR

SEQ ID NO: 208 - protein, 30 aa, Homo sapien
LEQEHNNLVNVPLCVDMCLNWLLNVYDTGR

SEQ ID NO: 209 - protein, 8 aa, Homo sapien
TGIISLCK

SEQ ID NO: 210 - protein, 6 aa, Homo sapien
AHLEDK

SEQ ID NO: 211 - protein, 12 aa, Homo sapien
QVASSTGFCDQR

SEQ ID NO: 212 - protein, 13 aa, Homo sapien
LGLLLHDSIQIPR

SEQ ID NO: 213 - protein, 18 aa, Homo sapien
QLGEVASFGGSNIEPSVR

SEQ ID NO: 214 - protein, 13 aa, Homo sapien
PEIEAALFLDWMR

SEQ ID NO: 215 - protein, 14 aa, Homo sapien
LEPQSMVWLPVLHR

SEQ ID NO: 216 - protein, 8 aa, Homo sapien
VAAAETAK

SEQ ID NO: 217 - protein, 8 aa, Homo sapien
ECPIIGFR

SEQ ID NO: 218 - protein, 15 aa, Homo sapien
HFNYDICQSCFFSGR

SEQ ID NO: 219 - protein, 19 aa, Homo sapien
MHYPMVEYCTPTTSGEDVR

SEQ ID NO: 220 - protein, 45 aa, Homo sapien
MGYLPVQTVLEGDNMETPVTLINFWPVDSAPASSPQLSHDDTHSR

SEQ ID NO: 221 - protein, 7 aa, Homo sapien
IEHYASR

SEQ ID NO: 222 - protein, 46 aa, Homo sapien
LAEMENSNGSYLNDSISPNESIDDEHLLIQHYCQSLNQDSPLSQPR

SEQ ID NO: 223 - protein, 14 aa, Homo sapien
SPAQILISLESEER

SEQ ID NO: 224 - protein, 10 aa, Homo sapien
ILADLEEENR

SEQ ID NO: 225 - protein, 8 aa, Homo sapien
NLQAEYDR

SEQ ID NO: 226 - protein, 6 aa, Homo sapien
QQHEHK

SEQ ID NO: 227 - protein, 20 aa, Homo sapien
GLSPLPSPPEMMPTSPQSPR

SEQ ID NO: 228 - protein, 9 aa, Homo sapien
DAELIAEAK

SEQ ID NO: 229 - protein, 9 aa, Homo sapien
MQILEDHNK

SEQ ID NO: 230 - protein, 8 aa, Homo sapien
QLESQLHR

SEQ ID NO: 231 - protein, 11 aa, Homo sapien
QLLEQPQAEAK

SEQ ID NO: 232 - protein, 15 aa, Homo sapien
VNGTTVSSPSTSLQR

SEQ ID NO: 233 - protein, 10 aa, Homo sapien
SDSSQPMLLR

SEQ ID NO: 234 - protein, 41 aa, Homo sapien
VVGSQTSDSMGEEDLLSPPQDTSTGLEEVMEQLNNSFPSSR

SEQ ID NO: 235 - protein, 35 aa, Homo sapien
WVLLQDQPDLAPGLTTIGASPTQTVTLVTQPVVTK

SEQ ID NO: 236 - protein, 15 aa, Homo sapien
LEMPSSLMLEVPTHR

SEQ ID NO: 237 - protein, 17 aa, Homo sapien
MGYLPVQTVLEGDNMET

SEQ ID NO: 238 - protein, 38 aa, Homo sapien
QSNLHSYVPSTYLTEITHVSQALLEVEQLLNAPDLCAK

SEQ ID NO: 239 - protein, 9 aa, Homo sapien
LEEQSDQWK

SEQ ID NO: 240 - protein, 20 aa, Homo sapien
MGYLPVQTVLEGDNMETDTM

SEQ ID NO: 241 - protein, 8 aa, Homo sapien
LQELTLER

SEQ ID NO: 242 - protein, 3685 aa, Homo sapien
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 243 - protein, 1325 aa, Artificial sequence
  Recombinant protein
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 244 - protein, 1192 aa, Artificial Sequence
  recombinant protein
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 245 - protein, 1270 aa, Artificial sequence
  recombinant protein
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHSYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 246 - protein, 23 aa, Homo sapien
AISKLEMPSSLMLEVPTHRLLQQ

SEQ ID NO: 247 - protein, 21 aa, Homo sapien
NWLKIQPTTPSEPTAIKSQLK

SEQ ID NO: 248 - protein, 21 aa, Homo sapien
HSKKTAALQSATPVERVKLQE

SEQ ID NO: 249 - protein, 21 aa, Homo sapien
KILRSLEGSDDAVLLQRRLDN

SEQ ID NO: 250 - protein, 21 aa, Homo sapien
ETVRIFLTEQPLEGLEKLYQE

SEQ ID NO: 251 - protein, 18 aa, Homo sapien
EWEKLNLHSADWQRKIDE

SEQ ID NO: 252 - protein, 17 aa, Homo sapien
TRWKLLQVAVEDRVRQL

SEQ ID NO: 253 - protein, 18 aa, Homo sapien
ELERILADLEEENRNLQA